(19) United States
(12) Patent Application Publication
(10) Pub. No.: US 2009/0304653 A1
(43) Pub. Date: Dec. 10, 2009

(54) METHODS TO IDENTIFY POLYNUCLEOTIDE AND POLYPEPTIDE SEQUENCES WHICH MAY BE ASSOCIATED WITH PHYSIOLOGICAL AND MEDICAL CONDITIONS

(75) Inventor: Walter Messier, Longmont, CO (US)

Correspondence Address: SWANSON & BRATSCHUN, L.L.C., 8210 SOUTHPARK TERRACE, LITTLETON, CO, 80120, US

(73) Assignee: EVOLUTIONARY GENOMICS, INC., Lafayette, CO (US)

(21) Appl. No.: 12/419,268

(22) Filed: Apr. 6, 2009

Related U.S. Application Data
(63) Continuation-in-part of application No. 11/781,818, filed on Jul. 23, 2007.
Application No. 11/781,818 is a continuation-in-part of application No. 10/883,576, filed on Jun. 30, 2004, now Pat. No. 7,247,425.
Application No. 10/883,576 is a continuation-in-part of application No. 10/098,600, filed on Mar. 14, 2002, now Pat. No. 6,866,996.
Application No. 10/098,600 is a continuation-in-part of application No. 09/942,252, filed on Aug. 28, 2001, now abandoned.
Application No. 09/942,252 is a continuation-in-part of application No. 09/591,435, filed on Jun. 9, 2000, now Pat. No. 6,280,953.
Application No. 09/591,435 is a continuation-in-part of application No. 09/240,915, filed on Jan. 29, 1999, now Pat. No. 6,228,586.
(60) Provisional application No. 61/042,603, filed on Apr. 4, 2008.
(60) Provisional application No. 60/545,604, filed on Feb. 17, 2004.
(60) Provisional application No. 60/484,030, filed on Jun. 30, 2003.
(60) Provisional application No. 60/098,987, filed on Sep. 2, 1998.
(60) Provisional application No. 60/073,263, filed on Jan. 30, 1998.

(51) Int. Cl. A61K 45/00; C12Q 1/70; C07K 14/00; C12N 5/10; G01N 33/566; G01N 33/53; A61P 31/18

(52) U.S. Cl. 424/93.21; 435/5; 530/350; 435/366; 424/93.2; 436/501; 435/7.21

(57) ABSTRACT

Disclosed are methods to identify an agent which may modulate resistance to HIV-1-mediated disease, comprising contacting at least one agent to be tested with a cell comprising human ICAM-1, and detecting the cell's resistance to HIV-1 viral replication, propagation, or function, wherein an agent is identified by its ability to increase the cell's resistance to HIV-1 viral replication, propagation, or function. Also disclosed are human mutant ICAM-1 polypeptides and methods to treat HIV-1 viral replication, propagation, or function in a human subject by ICAM-1 gene therapy relating to one or more of the following 10 mutations to human ICAM-1: L18Q, K29D, P45G, R49W, E171Q, wherein the mutant ICAM-1 is otherwise identical to human ICAM-1.

### CROSS-REFERENCE TO RELATED APPLICATIONS

This application claims priority to U.S. Provisional Patent Application No. 61/042,603 filed Apr. 4, 2008 and is a continuation in part of U.S. application Ser. No. 11/781,818, filed Jul. 23, 2007; which is a continuation-in-part of U.S. patent application Ser. No. 10/883,576, filed Jun. 30, 2004, now U.S. Pat. No. 7,247,427; U.S. application Ser. No. 10/883,576 claims priority to U.S. Provisional Patent Application No. 60/545,604 filed Feb. 17, 2004 and further claims priority to U.S. Provisional Patent Application No. 60/484,030 filed Jun. 30, 2003; U.S. application Ser. No. 10/883,576 is a continuation-in-part of U.S. application Ser. No. 10/098,600 filed Mar. 14, 2002, now U.S. Pat. No. 6,866,996; U.S. application Ser. No. 10/098,600 is a continuation-in-part of U.S. patent application Ser. No. 09/942,252 filed Aug. 28, 2001 (abandoned); U.S. application Ser. No. 09/942,252 is a continuation-in-part of U.S. patent application Ser. No. 09/591,435 filed Jun. 9, 2000, now U.S. Pat. No. 6,280,953; U.S. patent application Ser. No. 09/591,435 is a continuation-in-part of U.S. patent application Ser. No. 09/240,915 filed Jan. 29, 1999, now U.S. Pat. No. 6,228,586, which claims priority to U.S. Provisional Patent Application No. 60/098,987 filed Sep. 2, 1998, and further claims priority to U.S. Provisional Patent Application No. 60/073,263 filed Jan. 30, 1998, each of which is incorporated herein in its entirety.

### TECHNICAL FIELD

This invention relates to using molecular and evolutionary techniques to identify polynucleotide and polypeptide sequences corresponding to evolved traits that may be relevant to human diseases or conditions, such as unique or enhanced human brain functions, longer human life spans, susceptibility or resistance to development of infectious disease (such as AIDS and hepatitis C), susceptibility or resistance to development of cancer, and aesthetic traits, such as hair growth, susceptibility or resistance to acne, or enhanced muscle mass.

### BACKGROUND OF THE INVENTION

Humans differ from their closest evolutionary relatives, the non-human primates such as chimpanzees, in certain physiological and functional traits that relate to areas important to human health and well-being. For example, (1) humans have unique or enhanced brain function (e.g., cognitive skills, etc.) compared to chimpanzees; (2) humans have a longer life-span than non-human primates; (3) chimpanzees are resistant to certain infectious diseases that afflict humans, such as AIDS and hepatitis C; (4) chimpanzees appear to have a lower incidence of certain cancers than humans; (5) chimpanzees do not suffer from acne or alopecia (baldness); (6) chimpanzees have a higher percentage of muscle to fat; (7) chimpanzees are more resistant to malaria; (8) chimpanzees are less susceptible to Alzheimer=s disease; and (9) chimpanzees have a lower incidence of atherosclerosis. At the present time, the genes underlying the above human/chimpanzee differences are not known, nor, more importantly, are the specific changes that have evolved in these genes to provide these capabilities. Understanding the basis of these differences between humans and our close evolutionary relatives will provide useful information for developing effective treatments for related human conditions and diseases.

Classic evolution analysis, which compares mainly the anatomic features of animals, has revealed dramatic morphological and functional differences between human and non-human primates; yet, the human genome is known to share remarkable sequence similarities with that of other primates. For example, it is generally concluded that human DNA sequence is roughly 98.5% identical to chimpanzee DNA and only slightly less similar to gorilla DNA. McConkey and Goodman (1997) TIG 13:350-351. Given the relatively small percentage of genomic difference between humans and closely related primates, it is possible, if not likely, that a relatively small number of changes in genomic sequences may be responsible for traits of interest to human health and well-being, such as those listed above. Thus, it is desirable and feasible to identify the genes underlying these traits and to glean information from the evolved changes in the proteins they encode to develop treatments that could benefit human health and well-being. Identifying and characterizing these sequence changes is crucial in order to benefit from evolutionary solutions that have eliminated or minimized diseases or that provide unique or enhanced functions.

Recent developments in the human genome project have provided a tremendous amount of information on human gene sequences. Furthermore, the structures and activities of many human genes and their protein products have been studied either directly in human cells in culture or in several animal model systems, such as the nematode, fruit fly, zebrafish and mouse. These model systems have great advantages in being relatively simple, easy to manipulate, and having short generation times. Because the basic structures and biological activities of many important genes have been conserved throughout evolution, homologous genes can be identified in many species by comparing macromolecule sequences. Information obtained from lower species on important gene products and functional domains can be used to help identify the homologous genes or functional domains in humans. For example, the homeo domain with DNA binding activity first discovered in the fruit fly Drosophila was used to identify human homologues that possess similar activities.

Although comparison of homologous genes or proteins between human and a lower model organism may provide useful information with respect to evolutionarily conserved molecular sequences and functional features, this approach is of limited use in identifying genes whose sequences have changed due to natural selection. With the advent of the development of sophisticated algorithms and analytical methods, much more information can be teased out of DNA sequence changes. The most powerful of these methods, “K_{A}/K_{S}” involves pairwise comparisons between aligned protein-coding nucleotide sequences of the ratios of

$$\frac{ \begin{matrix}nonsynonymous nucleotide substitutions \\ per nonsynonymous site ( {K}_{A} )\end{matrix}}{synonymous substitutions per synonymous site ( {K}_{S} )}$$

(where nonsynonymous means substitutions that change the encoded amino acid and synonymous means substitutions that do not change the encoded amino acid). “K_{A}/K_{S}-type methods” includes this and similar methods. These methods have been used to demonstrate the occurrence of Darwinian molecular-level positive selection, resulting in amino acid differences in homologous proteins. Several groups have used such methods to document that a particular protein has evolved more rapidly than the neutral substitution rate, and thus supports the existence of Darwinian molecular-level positive selection. For example, McDonald and Kreitman (1991) Nature 351:652-654 propose a statistical test of neutral protein evolution hypothesis based on comparison of the number of amino acid replacement substitutions to synonymous substitutions in the coding region of a locus. When they apply this test to the Adh locus of three Drosophila species, they conclude that it shows instead that the locus has undergone adaptive fixation of selectively advantageous mutations and that selective fixation of adaptive mutations may be a viable alternative to the clocklike accumulation of neutral mutations as an explanation for most protein evolution. Jenkins et al. (1995) Proc. R. Soc. Lond. B 261:203-207 use the McDonald & Kreitman test to investigate whether adaptive evolution is occurring in sequences controlling transcription (non-coding sequences).

Nakashima et al. (1995) Proc. Natl. Acad. Sci. USA 92:5606-5609, use the method of Miyata and Yasunaga to perform pairwise comparisons of the nucleotide sequences of ten PLA2 isozyme genes from two snake species; this method involves comparing the number of nucleotide substitutions per site for the noncoding regions including introns (K_{N}) and the K_{A} and K_{S}. They conclude that the protein coding regions have been evolving at much higher rates than the noncoding regions including introns. The highly accelerated substitution rate is responsible for Darwinian molecular-level evolution of PLA2 isozyme genes to produce new physiological activities that must have provided strong selective advantage for catching prey or for defense against predators. Endo et al. (1996) Mol. Biol. Evol. 13(5):685-690 use the method of Nei and Gojobori, wherein d_{N} is the number of nonsynonymous substitutions and ds is the number of synonymous substitutions, for the purpose of identifying candidate genes on which positive selection operates. Metz and Palumbi (1996) Mol. Biol. Evol. 13(2):397-406 use the McDonald & Kreitman test as well as a method attributed to Nei and Gojobori, Nei and Jin, and Kumar, Tamura, and Nei; examining the average proportions of Pₙ, the replacement substitutions per replacement site, and Pₛ, the silent substitutions per silent site, to look for evidence of positive selection on bindin genes in sea urchins to investigate whether they have rapidly evolved as a prelude to species formation. Goodwin et al. (1996) Mol. Biol. Evol. 13(2):346-358 uses similar methods to examine the evolution of a particular murine gene family and conclude that the methods provide important fundamental insights into how selection drives genetic divergence in an experimentally manipulatable system. Edwards et al. (1995) use degenerate primers to pull out MHC loci from various species of birds and an alligator species, which are then analyzed by the Nei and Gojobori methods (d_{N}:d_{S} ratios) to extend MHC studies to nonmammalian vertebrates. Whitfield et al. (1993) Nature 364:713-715 use Ka/Ks analysis to look for directional selection in the regions flanking a conserved region in the SRY gene (that determines male sex). They suggest that the rapid evolution of SRY could be a significant cause of reproductive isolation, leading to new species. Wettsetin et al. (1996) Mol. Biol. Evol. 13(1):56-66 apply the MEGA program of Kumar, Tamura and Nei and phylogenetic analysis to investigate the diversification of MHC class I genes in squirrels and related rodents. Parham and Ohta (1996) Science 272:67-74 state that a population biology approach, including tests for selection as well as for gene conversion and neutral drift are required to analyze the generation and maintenance of human MHC class I polymorphism. Hughes (1997) Mol. Biol. Evol. 14(1): 1-5 compared over one hundred orthologous immunoglobulin C2 domains between human and rodent, using the method of Nei and Gojobori (d_{N}:d_{S} ratios) to test the hypothesis that proteins expressed in cells of the vertebrate immune system evolve unusually rapidly. Swanson and Vacquier (1998) Science 281:710-712 use d_{N}:d_{S} ratios to demonstrate concerted evolution between the lysin and the egg receptor for lysin and discuss the role of such concerted evolution in forming new species (speciation).

Due to the distant evolutionary relationships between humans and these lower animals, the adaptively valuable genetic changes fixed by natural selection are often masked by the accumulation of neutral, random mutations over time. Moreover, some proteins evolve in an episodic manner; such episodic changes could be masked, leading to inconclusive results, if the two genomes compared are not close enough. Messier and Stewart (1997) Nature 385:151-154. In fact, studies have shown that the occurrence of adaptive selection in protein evolution is often underestimated when predominantly distantly related sequences are compared. Endo et al. (1996) Mol. Biol. Evol. 37:441-456; Messier and Stewart (1997) Nature 385:151-154.

Molecular evolution studies within the primate family have been reported, but these mainly focus on the comparison of a small number of known individual genes and gene products to assess the rates and patterns of molecular changes and to explore the evolutionary mechanisms responsible for such changes. See generally, Li, Molecular Evolution, Sinauer Associates, Sunderland, Mass., 1997. Furthermore, sequence comparison data are used for phylogenetic analysis, wherein the evolution history of primates is reconstructed based on the relative extent of sequence similarities among examined molecules from different primates. For example, the DNA and amino acid sequence data for the enzyme lysozyme from different primates were used to study protein evolution in primates and the occurrence of adaptive selection within specific lineages. Malcolm et al. (1990) Nature 345:86-89; Messier and Stewart (1997). Other genes that have been subjected to molecular evolution studies in primates include hemoglobin, cytochrome c oxidase, and major histocompatibility complex (MHC). Nei and Hughes in: Evolution at the Molecular Level, Sinauer Associates, Sunderland, Mass. 222-247, 1991; Lienert and Parham (1996) Immunol. Cell Biol. 74:349-356; Wu et al. (1997) J. Mol. Evol. 44:477-491. Many non-coding sequences have also been used in molecular phylogenetic analysis of primates. Li, Molecular Evolution, Sinauer Associates, Sunderland, Mass. 1997. For example, the genetic distances among primate lineages were estimated from orthologous non-coding nucleotide sequences of beta-type globin loci and their flanking regions, and the evolution tree constructed for the nucleotide sequence orthologues depicted a branching pattern that is largely congruent with the picture from phylogenetic analyses of morphological characters. Goodman et al. (1990) J. Mol. Evol. 30:260-266.

Zhou and Li (1996) Mol. Biol. Evol. 13(6):780-783 applied K_{A}/K_{S} analysis to primate genes. It had previously been reported that gene conversion events likely have occurred in introns 2 and 4 between the red and green retinal pigment genes during human evolution. However, intron 4 sequences of the red and green retinal pigment genes from one European human were completely identical, suggesting a recent gene conversion event. In order to determine if the gene conversion event occurred in that individual, or a common ancestor of Europeans, or an even earlier hominid ancestor, the authors sequenced intron 4 of the red and green pigment gene from a male Asian human, a male chimpanzee, and a male baboon, and applied K_{A}/K_{S} analysis. They observed that the divergence between the two genes is significantly lower in intron 4 than in surrounding exons, suggesting that strong natural selection has acted against sequence homogenization.

Wolinsky et al. (1996) Science 272:537-542 used comparisons of nonsynonymous to synonymous base substitutions to demonstrate that the HIV virus itself (i.e., not the host species) is subject to adaptive evolution within individual human patients. Their goal was simply to document the occurrence of positive selection in a short time frame (that of a human patient=s course of disease). Niewiesk and Bangham (1996) J Mol Evol 42:452-458 used the Dₙ/Dₛ approach to ask a related question about the HTLV-1 virus, i.e., what are the selective forces acting on the virus itself. Perhaps because of an insufficient sample size, they were unable to resolve the nature of the selective forces. In both of these cases, although K_{A}/K_{S}-type methods were used in relation to a human virus, no attempt was made to use these methods for therapeutic goals (as in the present application), but rather to pursue narrow academic goals.

As can be seen from the papers cited above, analytical methods of molecular evolution to identify rapidly evolving genes (K_{A}/K_{S}-type methods) can be applied to achieve many different purposes, most commonly to confirm the existence of Darwinian molecular-level positive selection, but also to assess the frequency of Darwinian molecular-level positive selection, to understand phylogenetic relationships, to elucidate mechanisms by which new species are formed, or to establish single or multiple origin for specific gene polymorphisms. What is clear is from the papers cited above and others in the literature is that none of the authors applied K_{A}/K_{S}-type methods to identify evolutionary solutions, specific evolved changes, that could be mimicked or used in the development of treatments to prevent or cure human conditions or diseases or to modulate unique or enhanced human functions. They have not used K_{A}/K_{S} type analysis as a systematic tool for identifying human or non-human primate genes that contain evolutionarily significant sequence changes and exploiting such genes and the identified changes in the development of treatments for human conditions or diseases.

The identification of human genes that have evolved to confer unique or enhanced human functions compared to homologous chimpanzee genes could be applied to developing agents to modulate these unique human functions or to restore function when the gene is defective. The identification of the underlying chimpanzee (or other non-human primate) genes and the specific nucleotide changes that have evolved, and the further characterization of the physical and biochemical changes in the proteins encoded by these evolved genes, could provide valuable information, for example, on what determines susceptibility and resistance to infectious viruses, such as HIV and HCV, what determines susceptibility or resistance to the development of certain cancers, what determines susceptibility or resistance to acne, how hair growth can be controlled, and how to control the formation of muscle versus fat. This valuable information could be applied to developing agents that cause the human proteins to behave more like their chimpanzee homologues.

All references cited herein are hereby incorporated by reference in their entirety.

### SUMMARY OF THE INVENTION

The present invention provides methods for identifying polynucleotide and polypeptide sequences having evolutionarily significant changes which are associated with physiological conditions, including medical conditions. The invention applies comparative primate genomics to identify specific gene changes which may be associated with, and thus responsible for, physiological conditions, such as medically or commercially relevant evolved traits, and using the information obtained from these evolved genes to develop human treatments. The non-human primate sequences employed in the methods described herein may be any non-human primate, and are preferably a member of the hominoid group, more preferably a chimpanzee, bonobo, gorilla and/or orangutan, and most preferably a chimpanzee.

In one preferred embodiment, a non-human primate polynucleotide or polypeptide has undergone natural selection that resulted in a positive evolutionarily significant change (i.e., the non-human primate polynucleotide or polypeptide has a positive attribute not present in humans). In this embodiment the positively selected polynucleotide or polypeptide may be associated with susceptibility or resistance to certain diseases or with other commercially relevant traits. Examples of this embodiment include, but are not limited to, polynucleotides and polypeptides that are positively selected in non-human primates, preferably chimpanzees, that may be associated with susceptibility or resistance to infectious diseases and cancer. An example of a commercially relevant trait may include aesthetic traits such as hair growth, muscle mass, susceptibility or resistance to acne. An example of the disease resistance/susceptibility embodiment includes polynucleotides and polypeptides associated with the susceptibility or resistance to HIV dissemination, propagation and/or development of AIDS. The present invention can thus be useful in gaining insight into the molecular mechanisms that underlie resistance to HIV dissemination, propagation and/or development of AIDS, providing information that can also be useful in discovering and/or designing agents such as drugs that prevent and/or delay development of AIDS. Specific genes that have been positively selected in chimpanzees that may relate to AIDS or other infectious diseases are ICAM-1, ICAM-2, ICAM-3, MIP-1-α, CD59 and DC-SIGN. 17-β-hydroxysteroid dehydrogenase Type IV is a specific gene has been positively selected in chimpanzees that may relate to cancer. Additionally, the p44 gene is a gene that has been positively selected in chimpanzees and is believed to contribute to their HCV resistance.

In another preferred embodiment, a human polynucleotide or polypeptide has undergone natural selection that resulted in a positive evolutionarily significant change (i.e., the human polynucleotide or polypeptide has a positive attribute not present in non-human primates). One example of this embodiment is that the polynucleotide or polypeptide may be associated with unique or enhanced functional capabilities of the human brain compared to non-human primates. Another is the longer life-span of humans compared to non-human primates. A third is a commercially important aesthetic trait (e.g., normal or enhanced breast development). The present invention can thus be useful in gaining insight into the molecular mechanisms that underlie unique or enhanced human functions or physiological traits, providing information which can also be useful in designing agents such as drugs that modulate such unique or enhanced human functions or traits, and in designing treatment of diseases or conditions related to humans. As an example, the present invention can thus be useful in gaining insight into the molecular mechanisms that underlie human cognitive function, providing information which can also be useful in designing agents such as drugs that enhance human brain function, and in designing treatment of diseases related to the human brain. A specific example of a human gene that has positive evolutionarily significant changes when compared to non-human primates is a tyrosine kinase gene, the KIAA0641 or NM_{—}004920 gene.

Accordingly, in one aspect, the invention provides methods for identifying a polynucleotide sequence encoding a polypeptide, wherein said polypeptide may be associated with a physiological condition (such as a medically or commercially relevant positive evolutionarily significant change). The positive evolutionarily significant change can be found in humans or in non-human primates. In a preferred embodiment the invention provides a method for identifying a human AATYK polynucleotide sequence encoding a human AATYK polypeptide associated with an evolutionarily significant change. In another preferred embodiment, the invention provides a method for identifying a p44 polynucleotide and polypeptide that are associated with enhanced HCV resistance in chimpanzees relative to humans.

For any embodiment of this invention, the physiological condition may be any physiological condition, including those listed herein, such as, for example, disease (including susceptibility or resistance to disease) such as cancer, infectious disease (including viral diseases such as AIDS or HCV-associated chronic hepatitis); life span; brain function, including cognitive function or developmental sculpting; and aesthetic or cosmetic qualities, such as enhanced breast development.

In one aspect of the invention, methods are provided for identifying a polynucleotide sequence encoding a human polypeptide, wherein said polypeptide may be associated with a physiological condition that is present in human(s), comprising the steps of: a) comparing human protein-coding polynucleotide sequences to protein-coding polynucleotide sequences of a non-human primate, wherein the non-human primate does not have the physiological condition (or has it to a lesser degree); and b) selecting a human polynucleotide sequence that contains a nucleotide change as compared to corresponding sequence of the non-human primate, wherein said change is evolutionarily significant. In some embodiments, the human protein coding sequence (and/or the polypeptide encoded therein) may be associated with development and/or maintenance of a physiological condition or trait or a biological function. In some embodiments, the physiological condition or biological function may be life span, brain or cognitive function, or breast development (including adipose, gland and duct development). Methods used to assess the nucleotide change, and the nature(s) of the nucleotide change, are described herein, and apply to any and all embodiments. In a preferred embodiment, the method is a method for identifying a human AATYK polynucleotide sequence encoding a human AATYK polypeptide.

In other embodiments, methods are provided that comprise the steps of: (a) comparing human protein-coding nucleotide sequences to protein-coding nucleotide sequences of a non-human primate, preferably a chimpanzee, that is resistant to a particular medically relevant disease state, wherein the human protein coding sequence is or is believed to be associated with development of the disease; and (b) selecting a non-human polynucleotide sequence that contains at least one nucleotide change as compared to the corresponding sequence of the human, wherein the change is evolutionarily significant. The sequences identified by these methods may be further characterized and/or analyzed to confirm that they are associated with the development of the disease state or condition. The most preferred disease states that are applicable to these methods are cancer and infectious diseases, including AIDS, hepatitis C and leprosy.

In one embodiment, chimpanzee polynucleotide sequences are compared to human polynucleotide sequences to identify a p44 sequence that is evolutionarily significant. The p44 protein is (or is believed to be) associated with the enhanced HCV resistance of chimpanzees relative to humans.

In another aspect, methods are provided for identifying an evolutionarily significant change in a human brain polypeptide-coding polynucleotide sequence, comprising the steps of a) comparing human brain polypeptide-coding polynucleotide sequences to corresponding sequences of a non-human primate; and b) selecting a human polynucleotide sequence that contains a nucleotide change as compared to corresponding sequence of the non-human primate, wherein said change is evolutionarily significant. In some embodiments, the human brain polypeptide coding nucleotide sequences correspond to human brain cDNAs. In preferred embodiments, the human brain polypeptide-coding polynucleotide sequence is an AATYK sequence.

Another aspect of the invention includes methods for identifying a positively selected human evolutionarily significant change. These methods comprise the steps of: (a) comparing human polypeptide-coding nucleotide sequences to polypeptide-coding nucleotide sequences of a non-human primate; and (b) selecting a human polynucleotide sequence that contains at least one (i.e., one or more) nucleotide change as compared to corresponding sequence of the non-human primate, wherein said change is evolutionarily significant. The sequences identified by this method may be further characterized and/or analyzed for their possible association with biologically or medically relevant functions or traits unique or enhanced in humans. In preferred embodiments, the human polypeptide-coding nucleotide sequence is an AATYK sequence.

Another embodiment of the present invention is a method for large scale sequence comparison between human polypeptide-coding polynucleotide sequences and the polypeptide-coding polynucleotide sequences from a non-human primate, e.g., chimpanzee, comprising: (a) aligning the human polynucleotide sequences with corresponding polynucleotide sequences from non-human primate according to sequence homology; and (b) identifying any nucleotide changes within the human sequences as compared to the homologous sequences from the non-human primate, wherein the changes are evolutionarily significant. In some embodiments, the protein coding sequences are from brain.

In some embodiments, a nucleotide change identified by any of the methods described herein is a non-synonymous substitution. In some embodiments, the evolutionary significance of the nucleotide change is determined according to the non-synonymous substitution rate (K_{A}) of the nucleotide sequence. In some embodiments, the evolutionarily significant changes are assessed by determining the K_{A}/K_{S} ratio between the human gene and the homologous gene from non-human primate (such as chimpanzee), and preferably that ratio is at least about 0.75, more preferably greater than about 1 (unity) (i.e., at least about 1), more preferably at least about 1.25, more preferably at least about 1.50, and more preferably at least about 2.00. In other embodiments, once a positively selected gene has been identified between human and a non-human primate (such as chimpanzee or gorilla), further comparisons are performed with other non-human primates to confirm whether the human or the non-human primate (such as chimpanzee or gorilla) gene has undergone positive selection.

In another aspect, the invention provides methods for correlating an evolutionarily significant human nucleotide change to a physiological condition in a human (or humans), which comprise analyzing a functional effect (which includes determining the presence of a functional effect), if any, of (the presence or absence of) a polynucleotide sequence identified by any of the methods described herein, wherein presence of a functional effect indicates a correlation between the evolutionarily significant nucleotide change and the physiological condition. Alternatively, in these methods, a functional effect (if any) may be assessed using a polypeptide sequence (or a portion of the polypeptide sequence) encoded by a nucleotide sequence identified by any of the methods described herein.

In a preferred embodiment, the polynucleotide sequence or polypeptide sequence is a human or chimpanzee p44 polynucleotide sequence (SEQ ID NO. 34 OR 31) or polypeptide sequence (SEQ ID NO. 36 OR 33). In a more preferred embodiment, the p44 polynucleotide sequences are the exon 2 sequences having nucleotides 1-457 of SEQ ID NO:34 (human), and nucleotides 1-457 of SEQ ID NO:31 (chimpanzee), or fragments thereof containing the exon 2 evolutionarily significant chimpanzee nucleotides or the corresponding human nucleotides. Such fragments are preferably between 18 and 225 nucleotides in length.

The present invention also provides comparison of the identified polypeptides by physical and biochemical methods widely used in the art to determine the structural or biochemical consequences of the evolutionarily significant changes. Physical methods are meant to include methods that are used to examine structural changes to proteins encoded by genes found to have undergone adaptive evolution. Side-by-side comparison of the three-dimensional structures of a protein (either human or non-human primate) and the evolved homologous protein (either non-human primate or human, respectively) will provide valuable information for developing treatments for related human conditions and diseases. For example, using the methods of the present invention, the chimpanzee ICAM-1 gene (SEQ ID NO:85, FIG. 17) was identified as having positive evolutionary changes compared to human ICAM-1 (SEQ ID NO:1). In a three-dimensional model of two functional domains of the human ICAM-1 protein it can be seen that five of the six amino acids that have been changed in chimpanzees are immediately adjacent to (i.e., physically touching) amino acid residues known to be crucial for binding to the ICAM-1 counter-receptor, LFA-1; in each case, the human amino acid has been replaced by a larger amino acid in the chimpanzee ICAM-1. Such information allows insight into designing appropriate therapeutic intervention(s). Accordingly, in another aspect, the invention provides methods for identifying a target site (which includes one or more target sites) which may be suitable for therapeutic intervention, comprising comparing a human polypeptide (or a portion of the polypeptide) encoded in a sequence identified by any of the methods described herein, with a corresponding non-human polypeptide (or a portion of the polypeptide), wherein a location of a molecular difference, if any, indicates a target site.

Likewise, human and chimpanzee p44 polypeptide computer models or x-ray crystallography structures can be compared to determine how the evolutionarily significant amino acid changes of the chimpanzee p44 exon 2 alter the protein's structure, and how agents might be designed to interact with human p44 in such a manner that permits it to mimic chimpanzee p44 structure and/or function.

In another aspect, the invention provides methods for identifying a target site (which includes one or more target sites) which may be suitable for therapeutic intervention, comprising comparing a human polypeptide (or a portion of the polypeptide) encoded in a sequence identified by any of the methods described herein, with a corresponding non-human primate polypeptide (or a portion of the polypeptide), wherein a location of a molecular difference, such as an amino acid difference, if any, indicates a target site. Target sites can also be nonsynonymous nucleotide changes observed between a positively selected polynucleotide identified by any of the methods described herein and its corresponding sequence in the human or non-human primate. In preferred embodiments, the target site is a site on a human p44 polypeptide.

Biochemical methods are meant to include methods that are used to examine functional differences, such as binding specificity, binding strength, or optimal binding conditions, for a protein encoded by a gene that has undergone adaptive evolution. Side-by-side comparison of biochemical characteristics of a protein (either human or non-human primate) and the evolved homologous protein (either non-human primate or human, respectively) will reveal valuable information for developing treatments for related human conditions and diseases.

In another aspect, the invention provides methods of identifying an agent which may modulate a physiological condition, said method comprising contacting an agent (i.e., at least one agent to be tested) with a cell that has been transfected with a polynucleotide sequence identified by any of the methods described herein, wherein an agent is identified by its ability to modulate function of the polynucleotide sequence. In other embodiments, the invention provides methods of identifying an agent which may modulate a physiological condition, said method comprising contacting an agent (i.e., at least one agent) to be tested with a polypeptide (or a fragment of a polypeptide and/or a composition comprising a polypeptide or fragment of a polypeptide) encoded in or within a polynucleotide identified by any of the methods described herein, wherein an agent is identified by its ability to modulate function of the polypeptide. In preferred embodiments of these methods the polynucleotide sequence is an evolutionarily significant chimpanzee p44 polynucleotide sequence or its corresponding human polynucleotide. In more preferred embodiments, the polynucleotide sequence is nucleotides 1-457 of SEQ ID NO:31 (chimpanzee), and nucleotides 1-458 of SEQ ID NO:34 (human), or fragments thereof containing preferably 18-225 nucleotides and at least one of the chimpanzee evolutionarily significant nucleotides or corresponding human nucleotides. The invention also provides agents which are identified using the screening methods described herein.

In another aspect, the invention provides methods of screening agents which may modulate the activity of the human polynucleotide or polypeptide to either modulate a unique or enhanced human function or trait or to mimic the non-human primate trait of interest, such as susceptibility or resistance to development of a disease, such as HCV-associated chronic hepatitis or AIDS. These methods comprise contacting a cell which has been transfected with a polynucleotide sequence with an agent to be tested, and identifying agents based on their ability to modulate function of the polynucleotide or contacting a polypeptide preparation with an agent to be tested and identifying agents based upon their ability to modulate function of the polypeptide. In preferred embodiments, the polynucleotide sequence is an evolutionarily significant chimpanzee p44 polynucleotide sequence or its corresponding human polynucleotide sequence. In more preferred embodiments, the polynucleotide sequence is nucleotides 1-457 of SEQ ID NO: 31(chimpanzee), or nucleotides 1-457 of SEQ ID NO:34 (human), or fragments thereof containing preferably 18-225 nucleotides and at least one of the chimpanzee evolutionarily significant nucleotides or corresponding human nucleotides.

In another aspect of the invention, methods are provided for identifying candidate polynucleotides that may be associated with decreased resistance to development of a disease in humans, comprising comparing the human polynucleotide sequence with the corresponding non-human primate polynucleotide sequence to identify any nucleotide changes; and determining whether the human nucleotide changes are evolutionarily significant. It has been observed that human polynucleotides that are evolutionarily significant may, in some instances, be associated with increased susceptibility or decreased resistance to the development of human diseases such as cancer. As is described herein, the strongly positively selected BRCA1 gene's exon 11 is also the location of a number of mutations associated with breast, ovarian and/or prostate cancer. Thus, this phenomenon may represent a trade-off between enhanced development of one trait and loss or reduction in another trait in polynucleotides encoding polypeptides of multiple functions. In this way, identification of positively selected human polynucleotides can serve to identify a pool of genes that are candidates for susceptibility to human diseases.

Human candidate evolutionarily significant polynucleotides that are identified in this manner can be evaluated for their role in conferring susceptibility to diseases by analyzing the functional effect of the evolutionarily significant nucleotide change in the candidate polynucleotide in a suitable model system. The presence of a functional effect in the model system indicates a correlation between the nucleotide change in the candidate polynucleotide and the decreased resistance to development of the disease in humans. For example, if an evolutionarily significant polynucleotide containing all the evolutionarily significant nucleotide changes, or a similar polynucleotide with a lesser number of nucleotide changes, is found to increase the susceptibility to the disease at issue in a non-human primate model, this would be a functional effect that correlates the nucleotide change and the disease.

Alternatively, human candidate evolutionarily significant polynucleotides may, in some individuals, have mutations aside from the evolutionarily significant nucleotide changes, that confer the increased susceptibility to the disease. These mutations can be tested in a suitable model system for a functional effect, such as conversion to a neoplastic phenotype, to correlate the mutation to the disease.

Further, the subject method includes a diagnostic method to determine whether a human patient is predisposed to decreased resistance to the development of a disease, by assaying the patient's nucleic acids for the presence of a mutation in an evolutionarily significant polynucleotide, where the presence of the mutation in the polynucleotide has been determined by methods described herein as being diagnostic for decreased resistance to the development of the disease. In one embodiment, the polynucleotide is BRCA1 exon 11, and the disease is breast, prostate or ovarian cancer.

### BRIEF DESCRIPTION OF THE DRAWINGS

FIG. 1 depicts a phylogenetic tree for primates within the hominoid group. The branching orders are based on well-supported mitochondrial DNA phylogenies. Messier and Stewart (1997) Nature 385:151-154.

FIG. 2 (SEQ ID NOS:1-3) is a nucleotide sequence alignment between human and chimpanzee ICAM-1 sequences (GenBank® accession numbers X06990 and X86848, respectively). The amino acid translation of the chimpanzee sequence is shown below the alignment.

FIG. 3 shows the nucleotide sequence of gorilla ICAM-1 (SEQ ID NO:4).

FIG. 4 shows the nucleotide sequence of orangutan ICAM-1 (SEQ ID NO:5).

FIGS. 5(A)-(E) show the polypeptide sequence alignment of ICAM-1 from several primate species (SEQ ID NO:6).

FIGS. 6(A)-(B) show the polypeptide sequence alignment of ICAM-2 from several primate species (SEQ ID NO:7).

FIGS. 7(A)-(D) show the polypeptide sequence alignment of ICAM-3 from several primate species (SEQ ID NO:8).

FIG. 8 depicts a schematic representation of a procedure for comparing human/primate brain polynucleotides, selecting sequences with evolutionarily significant changes, and further characterizing the selected sequences. The diagram of FIG. 8 illustrates a preferred embodiment of the invention and together with the description serves to explain the principles of the invention, along with elaboration and optional additional steps. It is understood that any human/primate polynucleotide sequence can be compared by a similar procedure and that the procedure is not limited to brain polynucleotides.

FIG. 9 illustrates the known phylogenetic tree for the species compared in Example 14, with values of b, and b, mapped upon appropriate branches. Values of b, and b, were calculated by the method described in Zhang et al. (1998) Proc. Natl. Acad. Sci. USA 95:3708-3713. Values are shown above the branches; all values are shown 100×, for reasons of clarity. Statistical significance was calculated as for comparisons in Table 5 (Example 14), and levels of statistical significance are as shown as in Table 5. Note that only the branch leading from the human/chimpanzee common ancestor to modern humans shows a statistically significant value for b_{N}−b_{S}.

FIG. 10 illustrates a space-filling model of human CD59 with the duplicated GPI link (Asn) indicated by the darkest shading. This GPI link is duplicated in chimpanzees so that chimp CD59 contains 3 GPI links. The three areas of intermediate shading in FIG. 10 are other residues which differ between chimp and human.

FIG. 11 shows the coding sequence of human DC-SIGN (Genbank Acc. No. M98457) (SEQ. ID. NO. 9).

FIG. 12 shows the coding sequence of chimpanzee DC-SIGN (SEQ. ID. NO. 10).

FIG. 13 shows the coding sequence of gorilla DC-SIGN (SEQ. ID. NO. 11).

FIG. 14A shows the nucleotide sequence of the human AATYK gene. Start and stop codons are underlined (SEQ ID NO:14).

FIG. 14B shows an 1207 amino acid sequence of the human AATYK gene (SEQ ID NO:16).

FIG. 15A shows an 1806 base-pair region of the chimp AATYK gene (SEQ ID NO:17).

FIG. 15B shows an 1785 base-pair region of the gorilla AATYK gene (SEQ ID NO:18).

FIG. 16 shows a 1335 nucleotide region of the aligned chimpanzee (SEQ ID NO:31) and human (SEQ IS NO:34) p44 gene coding region. The underlined portion is exon 2, which was determined to be evolutionarily significant. Non-synonymous differences between the two sequences are indicated in bold, synonymous differences in italics. Chimpanzee has a single heterozygous base (position 212), shown as M (IUPAC code for A or C. The C base represents a nonsynonymous difference from human, while A is identical to the same position in the human homolog. Thus, these two chimpanzee alleles differ slightly in the K_{A}/K_{S} ratios relative to human p44.

FIG. 17 shows SEQ ID NO:85.

FIG. 18 shows RT PCR for expression of chICAM-1 and empty plasmid.

FIG. 19 shows p24 Concentration Indicative of HIV production level.

FIG. 20 shows TNF a levels in co-cultured cells.

FIG. 21 shows HIV production (left panel) and TNF a production (right panel) after 72 hours.

FIG. 22 shows HIV production at 24 (left) and 72 (right) hours in co-cultures of U937-1 and ACH2 cells.

FIG. 23 shows production of HIV (left) and TNF a (right) at different LPS concentrations.

FIG. 24 shows Chimpanzee-ICAM-1-expressing THP-1 cells were co-cultured with an equal number of ACH2 cells (a stable line of T-cells that constitutively express HIV-1). HIV-1 production was measured by an immunoassay for p24 in the cell supernatants after 24 and 72 hours.

FIG. 25 shows a cartoon of the crystal structure of dimerized domains 1 and 2 of ICAM 1.

### DETAILED DESCRIPTION OF THE INVENTION

The present invention applies comparative genomics to identify specific gene changes which are associated with, and thus may contribute to or be responsible for, physiological conditions, such as medically or commercially relevant evolved traits. The invention comprises a comparative genomics approach to identify specific gene changes responsible for differences in functions and diseases distinguishing humans from other non-humans, particularly primates, and most preferably chimpanzees, including the two known species, common chimpanzees and bonobos (pygmy chimpanzees). For example, chimpanzees and humans are 98.5% identical at the DNA sequence level and the present invention can identify the adaptive molecular changes underlying differences between the species in a number of areas, including unique or enhanced human cognitive abilities or physiological traits and chimpanzee resistance to HCV, AIDS and certain cancers. Unlike traditional genomics, which merely identifies genes, the present invention provides exact information on evolutionary solutions that eliminate disease or provide unique or enhanced functions or traits. The present invention identifies genes that have evolved to confer an evolutionary advantage and the specific evolved changes.

The present invention results from the observation that human protein-coding polynucleotides may contain sequence changes that are found in humans but not in other evolutionarily closely related species such as non-human primates, as a result of adaptive selection during evolution.

The present invention further results from the observation that the genetic information of non-human primates may contain changes that are found in a particular non-human primate but not in humans, as a result of adaptive selection during evolution. In this embodiment, a non-human primate polynucleotide or polypeptide has undergone natural selection that resulted in a positive evolutionarily significant change (i.e., the non-human primate polynucleotide or polypeptide has a positive attribute not present in humans). In this embodiment the positively selected polynucleotide or polypeptide may be associated with susceptibility or resistance to certain diseases or other commercially relevant traits. Medically relevant examples of this embodiment include, but are not limited to, polynucleotides and polypeptides that are positively selected in non-human primates, preferably chimpanzees, that may be associated with susceptibility or resistance to infectious diseases and cancer. An example of this embodiment includes polynucleotides and polypeptides associated with the susceptibility or resistance to progression from HIV infection to development of AIDS. The present invention can thus be useful in gaining insight into the molecular mechanisms that underlie resistance to progression from HIV infection to development of AIDS, providing information that can also be useful in discovering and/or designing agents such as drugs that prevent and/or delay development of AIDS. Likewise, the present invention can be useful in gaining insight into the underlying mechanisms for HCV resistance in chimpanzees as compared to humans. Commercially relevant examples include, but are not limited to, polynucleotides and polypeptides that are positively selected in non-human primates that may be associated with aesthetic traits, such as hair growth, absence of acne or muscle mass.

Positively selected human evolutionarily significant changes in polynucleotide and polypeptide sequences may be attributed to human capabilities that provide humans with competitive advantages, particularly when compared to the closest evolutionary relative, chimpanzee, such as unique or enhanced human brain functions. The present invention identifies human genes that evolved to provide unique or enhanced human cognitive abilities and the actual protein changes that confer functional differences will be quite useful in therapeutic approaches to treat cognitive deficiencies as well as cognitive enhancement for the general population.

Other positively selected human evolutionarily significant changes include those sequences that may be attributed to human physiological traits or conditions that are enhanced or unique relative to close evolutionary relatives, such as the chimpanzee, including enhanced breast development. The present invention provides a method of determining whether a polynucleotide sequence in humans that may be associated with enhanced breast development has undergone an evolutionarily significant change relative to a corresponding polynucleotide sequence in a closely related non-human primate. The identification of evolutionarily significant changes in the human polynucleotide that is involved in the development of unique or enhanced human physiological traits is important in the development of agents or drugs that can modulate the activity or function of the human polynucleotide or its encoded polypeptide.

The practice of the present invention employs, unless otherwise indicated, conventional techniques of molecular biology, genetics and molecular evolution, which are within the skill of the art. Such techniques are explained fully in the literature, such as: “Molecular Cloning: A Laboratory Manual”, second edition (Sambrook et al., 1989); “Oligonucleotide Synthesis” (M. J. Gait, ed., 1984); “Current Protocols in Molecular Biology” (F. M. Ausubel et al., eds., 1987); “PCR: The Polymerase Chain Reaction”, (Mullis et al., eds., 1994); “Molecular Evolution”, (Li, 1997).

### DEFINITIONS

As used herein, a “polynucleotide” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified polynucleotides such as methylated and/or capped polynucleotides. The terms “polynucleotide” and “nucleotide sequence” are used interchangeably.

As used herein, a “gene” refers to a polynucleotide or portion of a polynucleotide comprising a sequence that encodes a protein. It is well understood in the art that a gene also comprises non-coding sequences, such as 5= and 3=flanking sequences (such as promoters, enhancers, repressors, and other regulatory sequences) as well as introns.

The terms “polypeptide,” “peptide,” and “protein” are used interchangeably herein to refer to polymers of amino acids of any length. These terms also include proteins that are post-translationally modified through reactions that include glycosylation, acetylation and phosphorylation.

A “physiological condition” is a term well-understood in the art and means any condition or state that can be measured and/or observed. A “physiological condition” includes, but is not limited to, a physical condition, such as degree of body fat, alopecia (baldness), acne or enhanced breast development; life-expectancy; disease states (which include susceptibility and/or resistance to diseases), such as cancer or infectious diseases. Examples of physiological conditions are provided below (see, e.g., definitions of “human medically relevant medical condition”, “human commercially relevant condition”, “medically relevant evolved trait”, and “commercially relevant evolved trait”) and throughout the specification, and it is understood that these terms and examples refer to a physiological condition. A physiological condition may be, but is not necessarily, the result of multiple factors, any of which in turn may be considered a physiological condition. A physiological condition which is “present” in a human or non-human primate occurs within a given population, and includes those physiological conditions which are unique and/or enhanced in a given population when compared to another population.

The terms “human medically relevant condition” or “human commercially relevant condition” are used herein to refer to human conditions for which medical or non-medical intervention is desired.

The term “medically relevant evolved trait” is used herein to refer to traits that have evolved in humans or non-human primates whose analysis could provide information (e.g., physical or biochemical data) relevant to the development of a human medical treatment.

The term “commercially relevant evolved trait” is used herein to refer to traits that have evolved in humans or non-human primates whose analysis could provide information (e.g., physical or biochemical data) relevant to the development of a medical or non-medical product or treatment for human use.

The term “K_{A}/K_{S}-type methods” means methods that evaluate differences, frequently (but not always) shown as a ratio, between the number of nonsynonymous substitutions and synonymous substitutions in homologous genes (including the more rigorous methods that determine non-synonymous and synonymous sites). These methods are designated using several systems of nomenclature, including but not limited to K_{A}/K_{S}, d_{N}/d_{S}, D_{N}/D_{S}.

The terms “evolutionarily significant change” or “adaptive evolutionary change” refers to one or more nucleotide or peptide sequence change(s) between two species that may be attributed to a positive selective pressure. One method for determining the presence of an evolutionarily significant change is to apply a K_{A}/K_{S}-type analytical method, such as to measure a K_{A}/K_{S} ratio. Typically, a K_{A}/K_{S} ratio at least about 0.75, more preferably at least about 1.0, more preferably at least about 1.25, more preferably at least about 1.5 and most preferably at least about 2.0 indicates the action of positive selection and is considered to be an evolutionarily significant change.

Strictly speaking, only K_{A}/K_{S} ratios greater than 1.0 are indicative of positive selection. It is commonly accepted that the ESTs in GenBank® and other public databases often suffer from some degree of sequencing error, and even a few incorrect nucleotides can influence K_{A}/K_{S} scores. Thus, all pairwise comparisons that involve public ESTs must be undertaken with care. Due to the errors inherent in the publicly available databases, it is possible that these errors could depress a K_{A}/K_{S} ratio below 1.0. For this reason, K_{A}/K_{S} ratios between 0.75 and 1.0 should be examined carefully in order to determine whether or not a sequencing error has obscured evidence of positive selection. Such errors may be discovered through sequencing methods that are designed to be highly accurate.

The term “positive evolutionarily significant change” means an evolutionarily significant change in a particular species that results in an adaptive change that is positive as compared to other related species. Examples of positive evolutionarily significant changes are changes that have resulted in enhanced cognitive abilities or enhanced or unique physiological conditions in humans and adaptive changes in chimpanzees that have resulted in the ability of the chimpanzees infected with HIV or HCV to be resistant to progression of the infection.

The term “enhanced breast development” refers to the enlarged breasts observed in humans relative to non-human primates. The enlarged human breast has increased adipose, duct and/or gland tissue relative to other primates, and develops prior to first pregnancy and lactation.

The term “resistant” means that an organism, such as a chimpanzee, exhibits an ability to avoid, or diminish the extent of, a disease condition and/or development of the disease, preferably when compared to non-resistant organisms, typically humans. For example, a chimpanzee is resistant to certain impacts of HCV, HIV and other viral infections, and/or it does not develop the ultimate disease (chronic hepatitis or AIDS, respectively).

The term “susceptibility” means that an organism, such as a human, fails to avoid, or diminish the extent of, a disease condition and/or development of the disease condition, preferably when compared to an organism that is known to be resistant, such as a non-human primate, such as chimpanzee. For example, a human is susceptible to certain impacts of HCV, HIV and other viral infections and/or development of the ultimate disease (chronic hepatitis or AIDS).

It is understood that resistance and susceptibility vary from individual to individual, and that, for purposes of this invention, these terms also apply to a group of individuals within a species, and comparisons of resistance and susceptibility generally refer to overall, average differences between species, although intra-specific comparisons may be used.

The term “homologous” or “homologue” or “ortholog” is known and well understood in the art and refers to related sequences that share a common ancestor and is determined based on degree of sequence identity. These terms describe the relationship between a gene found in one species and the corresponding or equivalent gene in another species. For purposes of this invention homologous sequences are compared. “Homologous sequences” or “homologues” or “orthologs” are thought, believed, or known to be functionally related. A functional relationship may be indicated in any one of a number of ways, including, but not limited to, (a) degree of sequence identity; (b) same or similar biological function. Preferably, both (a) and (b) are indicated. The degree of sequence identity may vary, but is preferably at least 50% (when using standard sequence alignment programs known in the art), more preferably at least 60%, more preferably at least about 75%, more preferably at least about 85%. Homology can be determined using software programs readily available in the art, such as those discussed in Current Protocols in Molecular Biology (F. M. Ausubel et al., eds., 1987) Supplement 30, section 7.718, Table 7.71. Preferred alignment programs are MacVector (Oxford Molecular Ltd, Oxford, U.K.) and ALIGN Plus (Scientific and Educational Software, Pennsylvania). Another preferred alignment program is Sequencher (Gene Codes, Ann Arbor, Mich.), using default parameters.

The term “nucleotide change” refers to nucleotide substitution, deletion, and/or insertion, as is well understood in the art.

The term “human protein-coding nucleotide sequence” which is “associated with susceptibility to AIDS” as used herein refers to a human nucleotide sequence that encodes a protein that is associated with HIV dissemination (within the organism, i.e., intra-organism infectivity), propagation and/or development of AIDS. Due to the extensive research in the mechanisms underlying progression from HIV infection to the development of AIDS, a number of candidate human genes are believed or known to be associated with one or more of these phenomena. A polynucleotide (including any polypeptide encoded therein) sequence associated with susceptibility to AIDS is one which is either known or implicated to play a role in HIV dissemination, replication, and/or subsequent progression to full-blown AIDS. Examples of such candidate genes are provided below.

“AIDS resistant” means that an organism, such as a chimpanzee, exhibits an ability to avoid, or diminish the extent of, the result of HIV infection (such as propagation and dissemination) and/or development of AIDS, preferably when compared to AIDS-susceptible humans.

“Susceptibility” to AIDS means that an organism, such as a human, fails to avoid, or diminish the extent of, the result of HIV infection (such as propagation and dissemination) and/or development of AIDS, preferably when compared to an organism that is known to be AIDS resistant, such as a non-human primate, such as chimpanzee.

The term “human protein-coding nucleotide sequence” which is “associated with susceptibility to HCV infection” as used herein refers to a human nucleotide sequence that encodes a polypeptide that is associated with HCV dissemination (within the organism, i.e., intra-organism infectivity), propagation and/or development of chronic hepatitis. Candidate human genes are believed or known to be associated with human susceptibility to HCV infection. A polynucleotide (including any polypeptide encoded therein) sequence associated with susceptibility to chronic hepatitis is one which is either known or implicated to play a role in HCV dissemination, replication, and/or subsequent progression to chronic hepatitis or hepatocellular carcinoma. One example of a polynucleotide associated with susceptibility is human p44 exon 2.

“HCV resistant” means that an organism, such as a chimpanzee, exhibits an ability to avoid, or diminish the extent of, the result of HCV infection (such as propagation and dissemination) and/or development of chronic hepatitis, preferably when compared to HCV-susceptible humans.

“Susceptibility” to HCV infection means that an organism, such as a human, fails to avoid, or diminish the extent of, the result of HCV infection (such as propagation and dissemination) and/or development of chronic hepatitis, preferably when compared to an organism that is known to be HCV infection resistant, such as a non-human primate, such as chimpanzee.

The term “brain protein-coding nucleotide sequence” as used herein refers to a nucleotide sequence expressed in the brain that encodes a protein. One example of the “brain protein-coding nucleotide sequence” is a brain cDNA sequence.

As used herein, the term “brain functions unique or enhanced in humans” or “unique functional capabilities of the human brain” or “brain functional capability that is unique or enhanced in humans” refers to any brain function, either in kind or in degree, that is identified and/or observed to be enhanced in humans compared to other non-human primates. Such brain functions include, but are not limited to high capacity information processing, storage and retrieval capabilities, creativity, memory, language abilities, brain-mediated emotional response, locomotion, pain/pleasure sensation, olfaction, and temperament.

“Housekeeping genes” is a term well understood in the art and means those genes associated with general cell function, including but not limited to growth, division, stasis, metabolism, and/or death. “Housekeeping” genes generally perform functions found in more than one cell type. In contrast, cell-specific genes generally perform functions in a particular cell type (such as neurons) and/or class (such as neural cells).

The term “agent”, as used herein, means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

The term “to modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly.

A “function of a polynucleotide” includes, but is not limited to, replication; translation; and expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function.

A “function of a polypeptide” includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

The terms “modulate susceptibility to development of AIDS” and “modulate resistance to development of AIDS”, as used herein, include modulating intra-organism cell-to-cell transmission or infectivity of HIV. The terms further include reducing susceptibility to development of AIDS and/or cell-to-cell transmission or infectivity of HIV. The terms further include increasing resistance to development of AIDS and/or cell-to-cell transmission or infectivity of HIV. One means of assessing whether an agent is one that modulates susceptibility or resistance to development of AIDS is to determine whether at least one index of HIV susceptibility is affected, using a cell-based system as described herein, as compared with an appropriate control. Indicia of HIV susceptibility include, but are not limited to, cell-to-cell transmission of the virus, as measured by total number of cells infected with HIV and syncytia formation.

The terms “modulate susceptibility to HCV infection” and “modulate resistance to HCV infection”, as used herein, include modulating intra-organism cell-to-cell transmission or infectivity of HCV. The terms further include reducing susceptibility to development of chronic hepatitis and/or cell-to-cell transmission or infectivity of HCV. The terms further include increasing resistance to infection by HCV and/or cell-to-cell transmission or infectivity of HCV. One means of assessing whether an agent is one that modulates susceptibility or resistance to development of HCV-associated chronic hepatitis is to determine whether at least one index of HCV susceptibility is affected, using a cell-based system as described herein, as compared with an appropriate control. Indicia of HCV susceptibility include, but are not limited to, cell-to-cell transmission of the virus, as measured by total number of cells infected with HCV.

The term “target site” means a location in a polypeptide which can be one or more amino acids and/or is a part of a structural and/or functional motif, e.g., a binding site, a dimerization domain, or a catalytic active site. It also includes a location in a polynucleotide where there is one or more non-synonymous nucleotide changes in a protein coding region, or may also refer to a regulatory region of a positively selected gene. Target sites may be a useful for direct or indirect interaction with an agent, such as a therapeutic agent.

The term “molecular difference” includes any structural and/or functional difference. Methods to detect such differences, as well as examples of such differences, are described herein.

A “functional effect” is a term well known in the art, and means any effect which is exhibited on any level of activity, whether direct or indirect.

An agent that interacts with human p44 polypeptide to form a complex that “mimics the structure” of chimpanzee or other non-human primate p44 polypeptide means that the interaction of the agent with the human p44 polypeptide results in a complex whose three-dimensional structure more closely approximates the three-dimensional structure of the chimpanzee or non-human p44 polypeptide, relative to the human p44 polypeptide alone.

An agent that interacts with human p44 polypeptide to form a complex that “mimics the function” of chimpanzee or other non-human primate p44 polypeptide means that the complex of human p44 polypeptide and agent attain a biological function or enhance a biological function that is characteristic of the chimpanzee or other non-human primate p44 polypeptide, relative to the human p44 polypeptide alone. Such biological function of chimpanzee p44 polypeptide includes, without limitation, microtubule assembly following HCV infection, and resistance to HCV infection of hepatocytes.

### General Procedures Known in the Art

For the purposes of this invention, the source of the human and non-human polynucleotide can be any suitable source, e.g., genomic sequences or cDNA sequences. Preferably, cDNA sequences from human and a non-human primate are compared. Human protein-coding sequences can be obtained from public databases such as the Genome Sequence Data Bank and GenBank. These databases serve as repositories of the molecular sequence data generated by ongoing research efforts. Alternatively, human protein-coding sequences may be obtained from, for example, sequencing of cDNA reverse transcribed from mRNA expressed in human cells, or after PCR amplification, according to methods well known in the art. Alternatively, human genomic sequences may be used for sequence comparison. Human genomic sequences can be obtained from public databases or from a sequencing of commercially available human genomic DNA libraries or from genomic DNA, after PCR.

The non-human primate protein-coding sequences can be obtained by, for example, sequencing cDNA clones that are randomly selected from a non-human primate cDNA library. The non-human primate cDNA library can be constructed from total mRNA expressed in a primate cell using standard techniques in the art. In some embodiments, the cDNA is prepared from mRNA obtained from a tissue at a determined developmental stage, or a tissue obtained after the primate has been subjected to certain environmental conditions. cDNA libraries used for the sequence comparison of the present invention can be constructed using conventional cDNA library construction techniques that are explained fully in the literature of the art. Total mRNAs are used as templates to reverse-transcribe cDNAs. Transcribed cDNAs are subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al. (1996) Genome Research 6:791-806. cDNA clones randomly selected from the constructed cDNA library can be sequenced using standard automated sequencing techniques. Preferably, full-length cDNA clones are used for sequencing. Either the entire or a large portion of cDNA clones from a cDNA library may be sequenced, although it is also possible to practice some embodiments of the invention by sequencing as little as a single cDNA, or several cDNA clones.

In one preferred embodiment of the present invention, non-human primate cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same animal. Under certain hybridization conditions with appropriate stringency and concentration, those cDNAs that hybridize with non-tissue specific mRNAs and thus likely represent “housekeeping” genes will be excluded from the cDNA pool. Accordingly, remaining cDNAs to be sequenced are more likely to be associated with tissue-specific functions. For the purpose of subtraction hybridization, non-tissue-specific mRNAs can be obtained from one organ, or preferably from a combination of different organs and cells. The amount of non-tissue-specific mRNAs are maximized to saturate the tissue-specific cDNAs.

Alternatively, information from online public databases can be used to select or give priority to cDNAs that are more likely to be associated with specific functions. For example, the non-human primate cDNA candidates for sequencing can be selected by PCR using primers designed from candidate human cDNA sequence. Candidate human cDNA sequences are, for example, those that are only found in a specific tissue, such as brain or breast, or that correspond to genes likely to be important in the specific function, such as brain function or breast tissue adipose or glandular development. Such human tissue-specific cDNA sequences can be obtained by searching online human sequence databases such as GenBank, in which information with respect to the expression profile and/or biological activity for cDNA sequences are specified.

Sequences of non-human primate (for example, from an AIDS- or HCV-resistant non-human primate) homologue(s) to a known human gene may be obtained using methods standard in the art, such as from public databases such as GenBank or PCR methods (using, for example, GeneAmp PCR System 9700 thermocyclers (Applied Biosystems, Inc.)). For example non-human primate cDNA candidates for sequencing can be selected by PCR using primers designed from candidate human cDNA sequences. For PCR, primers may be made from the human sequences using standard methods in the art, including publicly available primer design programs such as PRIMER7 (Whitehead Institute). The sequence amplified may then be sequenced using standard methods and equipment in the art, such as automated sequencers (Applied Biosystems, Inc.).

### General Methods of the Invention

The general method of the invention is as follows. Briefly, nucleotide sequences are obtained from a human source and a non-human source. The human and non-human nucleotide sequences are compared to one another to identify sequences that are homologous. The homologous sequences are analyzed to identify those that have nucleic acid sequence differences between the two species. Then molecular evolution analysis is conducted to evaluate quantitatively and qualitatively the evolutionary significance of the differences. For genes that have been positively selected between two species, e.g., human and chimp, it is useful to determine whether the difference occurs in other non-human primates. Next, the sequence is characterized in terms of molecular/genetic identity and biological function. Finally, the information can be used to identify agents useful in diagnosis and treatment of human medically or commercially relevant conditions.

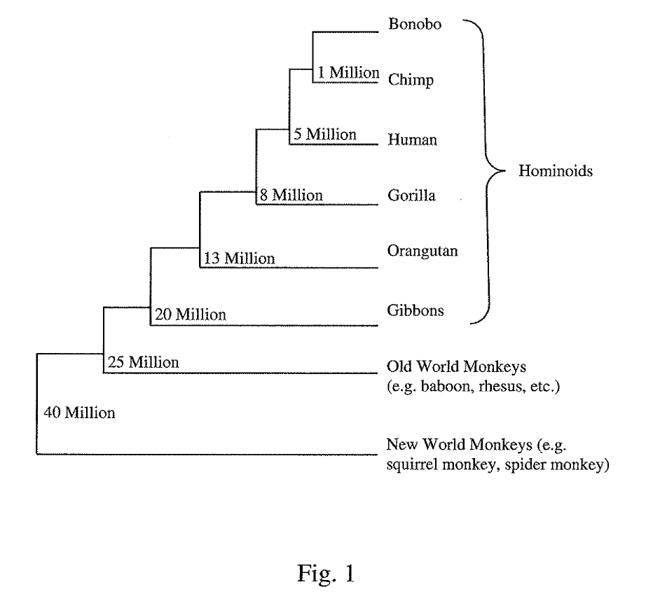

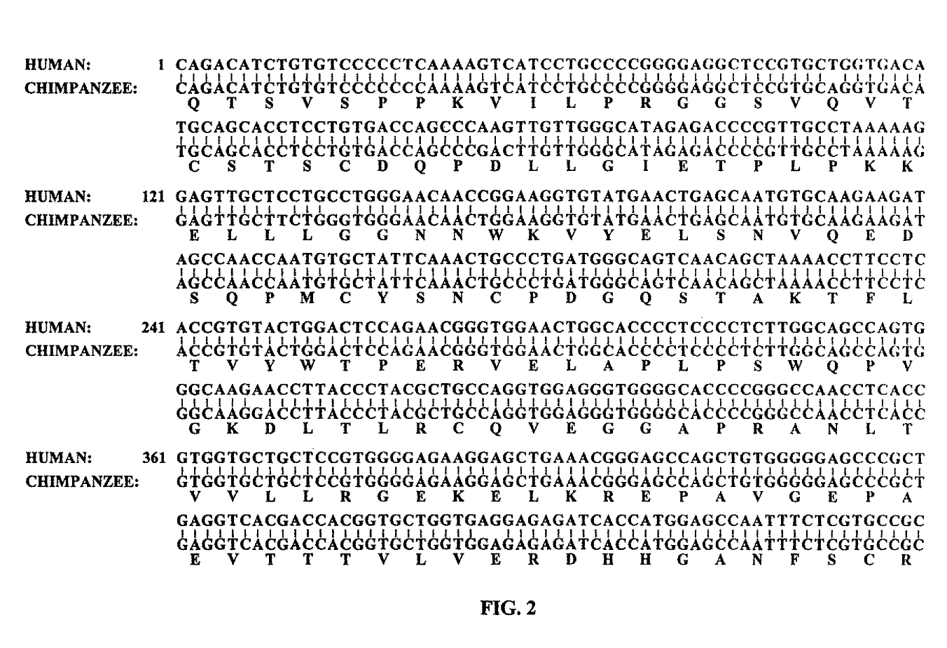

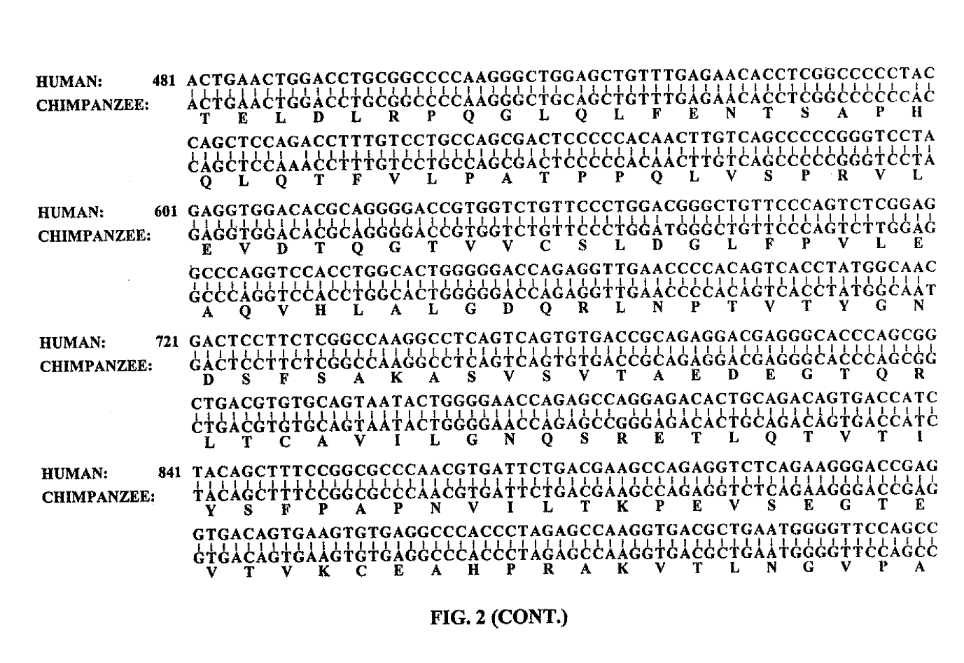

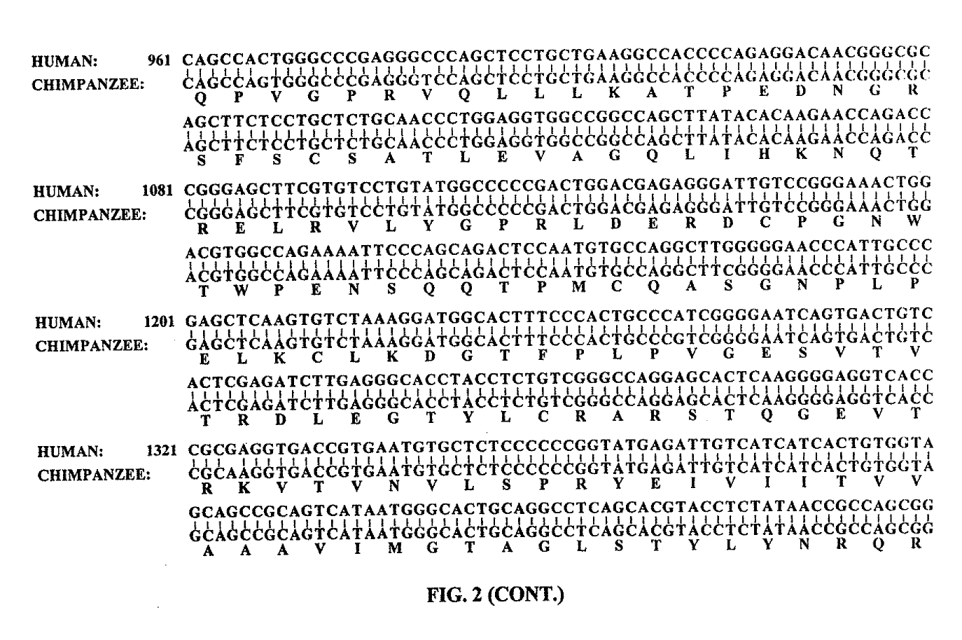

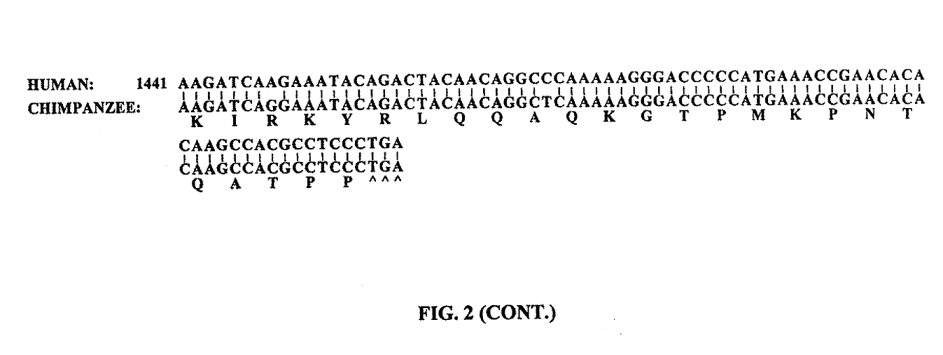

The general methods of the invention entail comparing human protein-coding nucleotide sequences to protein-coding nucleotide sequences of a non-human, preferably a primate, and most preferably a chimpanzee. Examples of other non-human primates are bonobo, gorilla, orangutan, gibbon, Old World monkeys, and New World monkeys. A phylogenetic tree for primates within the hominoid group is depicted in FIG. 1. Bioinformatics is applied to the comparison and sequences are selected that contain a nucleotide change or changes that is/are evolutionarily significant change(s). The invention enables the identification of genes that have evolved to confer some evolutionary advantage and the identification of the specific evolved changes.

Protein-coding sequences of human and another non-human primate are compared to identify homologous sequences. Protein-coding sequences known to or suspected of having a specific biological function may serve as the starting point for the comparison. Any appropriate mechanism for completing this comparison is contemplated by this invention. Alignment may be performed manually or by software (examples of suitable alignment programs are known in the art). Preferably, protein-coding sequences from a non-human primate are compared to human sequences via database searches, e.g., BLAST searches. The high scoring “hits,” i.e., sequences that show a significant similarity after BLAST analysis, will be retrieved and analyzed. Sequences showing a significant similarity can be those having at least about 60%, at least about 75%, at least about 80%, at least about 85%, or at least about 90% sequence identity. Preferably, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. (1992) CABIOS 8:189-191.

Alternatively, the sequencing and homologous comparison of protein-coding sequences between human and a non-human primate may be performed simultaneously by using the newly developed sequencing chip technology. See, for example, Rava et al. U.S. Pat. No. 5,545,531.

The aligned protein-coding sequences of human and another non-human primate are analyzed to identify nucleotide sequence differences at particular sites. Again, any suitable method for achieving this analysis is contemplated by this invention. If there are no nucleotide sequence differences, the non-human primate protein coding sequence is not usually further analyzed. The detected sequence changes are generally, and preferably, initially checked for accuracy. Preferably, the initial checking comprises performing one or more of the following steps, any and all of which are known in the art: (a) finding the points where there are changes between the non-human primate and human sequences; (b) checking the sequence fluorogram (chromatogram) to determine if the bases that appear unique to non-human primate correspond to strong, clear signals specific for the called base; (c) checking the human hits to see if there is more than one human sequence that corresponds to a sequence change. Multiple human sequence entries for the same gene that have the same nucleotide at a position where there is a different nucleotide in a non-human primate sequence provides independent support that the human sequence is accurate, and that the change is significant. Such changes are examined using public database information and the genetic code to determine whether these nucleotide sequence changes result in a change in the amino acid sequence of the encoded protein. As the definition of “nucleotide change” makes clear, the present invention encompasses at least one nucleotide change, either a substitution, a deletion or an insertion, in a human protein-coding polynucleotide sequence as compared to corresponding sequence from a non-human primate. Preferably, the change is a nucleotide substitution. More preferably, more than one substitution is present in the identified human sequence and is subjected to molecular evolution analysis.

Any of several different molecular evolution analyses or K_{A}/K_{S}-type methods can be employed to evaluate quantitatively and qualitatively the evolutionary significance of the identified nucleotide changes between human gene sequences and that of a non-human primate. Kreitman and Akashi (1995) Annu. Rev. Ecol. Syst. 26:403-422; Li, Molecular Evolution, Sinauer Associates, Sunderland, Mass., 1997. For example, positive selection on proteins (i.e., molecular-level adaptive evolution) can be detected in protein-coding genes by pairwise comparisons of the ratios of nonsynonymous nucleotide substitutions per nonsynonymous site (K_{A}) to synonymous substitutions per synonymous site (K_{S}) (Li et al., 1985; Li, 1993). Any comparison of K_{A} and K_{S} may be used, although it is particularly convenient and most effective to compare these two variables as a ratio. Sequences are identified by exhibiting a statistically significant difference between K_{A} and K_{S} using standard statistical methods.

Preferably, the K_{A}/K_{S} analysis by Li et al. is used to carry out the present invention, although other analysis programs that can detect positively selected genes between species can also be used. Li et al. (1985) Mol. Biol. Evol. 2:150-174; Li (1993); see also J. Mol. Evol. 36:96-99; Messier and Stewart (1997) Nature 385:151-154; Nei (1987) Molecular Evolutionary Genetics (New York, Columbia University Press). The K_{A}/K_{S} method, which comprises a comparison of the rate of non-synonymous substitutions per non-synonymous site with the rate of synonymous substitutions per synonymous site between homologous protein-coding region of genes in terms of a ratio, is used to identify sequence substitutions that may be driven by adaptive selections as opposed to neutral selections during evolution. A synonymous (“silent”) substitution is one that, owing to the degeneracy of the genetic code, makes no change to the amino acid sequence encoded; a non-synonymous substitution results in an amino acid replacement. The extent of each type of change can be estimated as K_{A} and K_{S}, respectively, the numbers of synonymous substitutions per synonymous site and non-synonymous substitutions per non-synonymous site. Calculations of K_{A}/K_{S} may be performed manually or by using software. An example of a suitable program is MEGA (Molecular Genetics Institute, Pennsylvania State University).

For the purpose of estimating K_{A} and K_{S}, either complete or partial human protein-coding sequences are used to calculate total numbers of synonymous and non-synonymous substitutions, as well as non-synonymous and synonymous sites. The length of the polynucleotide sequence analyzed can be any appropriate length. Preferably, the entire coding sequence is compared, in order to determine any and all significant changes. Publicly available computer programs, such as Li93 (Li (1993) J. Mol. Evol. 36:96-99) or INA, can be used to calculate the K_{A} and K_{S} values for all pairwise comparisons. This analysis can be further adapted to examine sequences in a “sliding window” fashion such that small numbers of important changes are not masked by the whole sequence. “Sliding window” refers to examination of consecutive, overlapping subsections of the gene (the subsections can be of any length).

The comparison of non-synonymous and synonymous substitution rates is represented by the K_{A}/K_{S} ratio. K_{A}/K_{S} has been shown to be a reflection of the degree to which adaptive evolution has been at work in the sequence under study. Full length or partial segments of a coding sequence can be used for the K_{A}/K_{S} analysis. The higher the K_{A}/K_{S} ratio, the more likely that a sequence has undergone adaptive evolution and the non-synonymous substitutions are evolutionarily significant. See, for example, Messier and Stewart (1997). Preferably, the K_{A}/K_{S} ratio is at least about 0.75, more preferably at least about 1.0, more preferably at least about 1.25, more preferably at least about 1.50, or more preferably at least about 2.00. Preferably, statistical analysis is performed on all elevated K_{A}/K_{S} ratios, including, but not limited to, standard methods such as Student=s t-test and likelihood ratio tests described by Yang (1998) Mol. Biol. Evol. 37:441-456.

K_{A}/K_{S} ratios significantly greater than unity strongly suggest that positive selection has fixed greater numbers of amino acid replacements than can be expected as a result of chance alone, and is in contrast to the commonly observed pattern in which the ratio is less than or equal to one. Nei (1987); Hughes and Hei (1988) Nature 335:167-170; Messier and Stewart (1994) Current Biol. 4:911-913; Kreitman and Akashi (1995) Ann. Rev. Ecol. Syst. 26:403-422; Messier and Stewart (1997). Ratios less than one generally signify the role of negative, or purifying selection: there is strong pressure on the primary structure of functional, effective proteins to remain unchanged.

All methods for calculating K_{A}/K_{S} ratios are based on a pairwise comparison of the number of nonsynonymous substitutions per nonsynonymous site to the number of synonymous substitutions per synonymous site for the protein-coding regions of homologous genes from related species. Each method implements different corrections for estimating “multiple hits” (i.e., more than one nucleotide substitution at the same site). Each method also uses different models for how DNA sequences change over evolutionary time. Thus, preferably, a combination of results from different algorithms is used to increase the level of sensitivity for detection of positively-selected genes and confidence in the result.

Preferably, K_{A}/K_{S} ratios should be calculated for orthologous gene pairs, as opposed to paralogous gene pairs (i.e., a gene which results from speciation, as opposed to a gene that is the result of gene duplication) Messier and Stewart (1997). This distinction may be made by performing additional comparisons with other non-human primates, such as gorilla and orangutan, which allows for phylogenetic tree-building. Orthologous genes when used in tree-building will yield the known “species tree”, i.e., will produce a tree that recovers the known biological tree. In contrast, paralogous genes will yield trees which will violate the known biological tree.

It is understood that the methods described herein could lead to the identification of human polynucleotide sequences that are functionally related to human protein-coding sequences. Such sequences may include, but are not limited to, non-coding sequences or coding sequences that do not encode human proteins. These related sequences can be, for example, physically adjacent to the human protein-coding sequences in the human genome, such as introns or 5=- and 3=-flanking sequences (including control elements such as promoters and enhancers). These related sequences may be obtained via searching a public human genome database such as GenBank or, alternatively, by screening and sequencing a human genomic library with a protein-coding sequence as probe. Methods and techniques for obtaining non-coding sequences using related coding sequence are well known to one skilled in the art.

The evolutionarily significant nucleotide changes, which are detected by molecular evolution analysis such as the K_{A}/K_{S} analysis, can be further assessed for their unique occurrence in humans (or the non-human primate) or the extent to which these changes are unique in humans (or the non-human primate). For example, the identified changes can be tested for presence/absence in other non-human primate sequences. The sequences with at least one evolutionarily significant change between human and one non-human primate can be used as primers for PCR analysis of other non-human primate protein-coding sequences, and resulting polynucleotides are sequenced to see whether the same change is present in other non-human primates. These comparisons allow further discrimination as to whether the adaptive evolutionary changes are unique to the human lineage as compared to other non-human primates or whether the adaptive change is unique to the non-human primates (i.e., chimpanzee) as compared to humans and other non-human primates. A nucleotide change that is detected in human but not other primates more likely represents a human adaptive evolutionary change. Alternatively, a nucleotide change that is detected in a non-human primate (i.e., chimpanzee) that is not detected in humans or other non-human primates likely represents a chimpanzee adaptive evolutionary change. Other non-human primates used for comparison can be selected based on their phylogenetic relationships with human. Closely related primates can be those within the hominoid sublineage, such as chimpanzee, bonobo, gorilla, and orangutan. Non-human primates can also be those that are outside the hominoid group and thus not so closely related to human, such as the Old World monkeys and New World monkeys. Statistical significance of such comparisons may be determined using established available programs, e.g., t-test as used by Messier and Stewart (1997) Nature 385:151-154. Those genes showing statistically high K_{A}/K_{S} ratios are very likely to have undergone adaptive evolution.

Sequences with significant changes can be used as probes in genomes from different human populations to see whether the sequence changes are shared by more than one human population. Gene sequences from different human populations can be obtained from databases made available by, for example, the Human Genome Project, the human genome diversity project or, alternatively, from direct sequencing of PCR-amplified DNA from a number of unrelated, diverse human populations. The presence of the identified changes in different human populations would further indicate the evolutionary significance of the changes. Chimpanzee sequences with significant changes can be obtained and evaluated using similar methods to determine whether the sequence changes are shared among many chimpanzees.

Sequences with significant changes between species can be further characterized in terms of their molecular/genetic identities and biological functions, using methods and techniques known to those of ordinary skill in the art. For example, the sequences can be located genetically and physically within the human genome using publicly available bioinformatics programs. The newly identified significant changes within the nucleotide sequence may suggest a potential role of the gene in human evolution and a potential association with human-unique functional capabilities. The putative gene with the identified sequences may be further characterized by, for example, homologue searching. Shared homology of the putative gene with a known gene may indicate a similar biological role or function. Another exemplary method of characterizing a putative gene sequence is on the basis of known sequence motifs. Certain sequence patterns are known to code for regions of proteins having specific biological characteristics such as signal sequences, DNA binding domains, or transmembrane domains.

The identified human sequences with significant changes can also be further evaluated by looking at where the gene is expressed in terms of tissue- or cell type-specificity. For example, the identified coding sequences can be used as probes to perform in situ mRNA hybridization that will reveal the expression patterns of the sequences. Genes that are expressed in certain tissues may be better candidates as being associated with important human functions associated with that tissue, for example brain tissue. The timing of the gene expression during each stage of human development can also be determined.

As another exemplary method of sequence characterization, the functional roles of the identified nucleotide sequences with significant changes can be assessed by conducting functional assays for different alleles of an identified gene in a model system, such as yeast, nematode, Drosophila, and mouse. Model systems may be cell-based or in vivo, such as transgenic animals or animals with chimeric organs or tissues. Preferably, the transgenic mouse or chimeric organ mouse system is used. Methods of making cell-based systems and/or transgenic/chimeric animal systems are known in the art and need not be described in detail herein.

As another exemplary method of sequence characterization, the use of computer programs allows modeling and visualizing the three-dimensional structure of the homologous proteins from human and chimpanzee. Specific, exact knowledge of which amino acids have been replaced in a primate's protein(s) allows detection of structural changes that may be associated with functional differences. Thus, use of modeling techniques is closely associated with identification of functional roles discussed in the previous paragraph. The use of individual or combinations of these techniques constitutes part of the present invention. For example, chimpanzee ICAM-3 contains a glutamine residue (Q101) at the site in which human ICAM-3 contains a proline (P101). The human protein is known to bend sharply at this point. Replacement of the proline by glutamine in the chimpanzee protein is likely to result in a much less sharp bend at this point. This has clear implications for packaging of the ICAM-3 chimpanzee protein into HIV virions.

Likewise, chimpanzee p44 has been found to contain an exon (exon2) having several evolutionarily significant nucleotide changes relative to human p44 exon 2. The nonsynonymous changes and corresponding amino acid changes in chimpanzee p44 polypeptide are believed to confer HCV resistance to the chimpanzee. The mechanism may involve enhanced p44 microtubule assembly in hepatocytes.

The sequences identified by the methods described herein have significant uses in diagnosis and treatment of medically or commercially relevant human conditions. Accordingly, the present invention provides methods for identifying agents that are useful in modulating human-unique or human-enhanced functional capabilities and/or correcting defects in these capabilities using these sequences. These methods employ, for example, screening techniques known in the art, such as in vitro systems, cell-based expression systems and transgenic/chimeric animal systems. The approach provided by the present invention not only identifies rapidly evolved genes, but indicates modulations that can be made to the protein that may not be too toxic because they exist in another species.

### Screening Methods

The present invention also provides screening methods using the polynucleotides and polypeptides identified and characterized using the above-described methods. These screening methods are useful for identifying agents which may modulate the function(s) of the polynucleotides or polypeptides in a manner that would be useful for a human treatment. Generally, the methods entail contacting at least one agent to be tested with either a cell that has been transfected with a polynucleotide sequence identified by the methods described above, or a preparation of the polypeptide encoded by such polynucleotide sequence, wherein an agent is identified by its ability to modulate function of either the polynucleotide sequence or the polypeptide.

As used herein, the term “agent” means a biological or chemical compound such as a simple or complex organic or inorganic molecule, a peptide, a protein or an oligonucleotide. A vast array of compounds can be synthesized, for example oligomers, such as oligopeptides and oligonucleotides, and synthetic organic and inorganic compounds based on various core structures, and these are also included in the term “agent”. In addition, various natural sources can provide compounds for screening, such as plant or animal extracts, and the like. Compounds can be tested singly or in combination with one another.

To “modulate function” of a polynucleotide or a polypeptide means that the function of the polynucleotide or polypeptide is altered when compared to not adding an agent. Modulation may occur on any level that affects function. A polynucleotide or polypeptide function may be direct or indirect, and measured directly or indirectly. A “function” of a polynucleotide includes, but is not limited to, replication, translation, and expression pattern(s). A polynucleotide function also includes functions associated with a polypeptide encoded within the polynucleotide. For example, an agent which acts on a polynucleotide and affects protein expression, conformation, folding (or other physical characteristics), binding to other moieties (such as ligands), activity (or other functional characteristics), regulation and/or other aspects of protein structure or function is considered to have modulated polynucleotide function. The ways that an effective agent can act to modulate the expression of a polynucleotide include, but are not limited to 1) modifying binding of a transcription factor to a transcription factor responsive element in the polynucleotide; 2) modifying the interaction between two transcription factors necessary for expression of the polynucleotide; 3) altering the ability of a transcription factor necessary for expression of the polynucleotide to enter the nucleus; 4) inhibiting the activation of a transcription factor involved in transcription of the polynucleotide; 5) modifying a cell-surface receptor which normally interacts with a ligand and whose binding of the ligand results in expression of the polynucleotide; 6) inhibiting the inactivation of a component of the signal transduction cascade that leads to expression of the polynucleotide; and 7) enhancing the activation of a transcription factor involved in transcription of the polynucleotide.

A “function” of a polypeptide includes, but is not limited to, conformation, folding (or other physical characteristics), binding to other moieties (such as ligands), activity (or other functional characteristics), and/or other aspects of protein structure or functions. For example, an agent that acts on a polypeptide and affects its conformation, folding (or other physical characteristics), binding to other moieties (such as ligands), activity (or other functional characteristics), and/or other aspects of protein structure or functions is considered to have modulated polypeptide function. The ways that an effective agent can act to modulate the function of a polypeptide include, but are not limited to 1) changing the conformation, folding or other physical characteristics; 2) changing the binding strength to its natural ligand or changing the specificity of binding to ligands; and 3) altering the activity of the polypeptide.

A “function” of a polynucleotide includes its expression, i.e., transcription and/or translation. It can also include (without limitation) its conformation, folding and binding to other moieties.

Generally, the choice of agents to be screened is governed by several parameters, such as the particular polynucleotide or polypeptide target, its perceived function, its three-dimensional structure (if known or surmised), and other aspects of rational drug design. Techniques of combinatorial chemistry can also be used to generate numerous permutations of candidates. Those of skill in the art can devise and/or obtain suitable agents for testing.

The in vivo screening assays described herein may have several advantages over conventional drug screening assays: 1) if an agent must enter a cell to achieve a desired therapeutic effect, an in vivo assay can give an indication as to whether the agent can enter a cell; 2) an in vivo screening assay can identify agents that, in the state in which they are added to the assay system are ineffective to elicit at least one characteristic which is associated with modulation of polynucleotide or polypeptide function, but that are modified by cellular components once inside a cell in such a way that they become effective agents; 3) most importantly, an in vivo assay system allows identification of agents affecting any component of a pathway that ultimately results in characteristics that are associated with polynucleotide or polypeptide function.

In general, screening can be performed by adding an agent to a sample of appropriate cells which have been transfected with a polynucleotide identified using the methods of the present invention, and monitoring the effect, i.e., modulation of a function of the polynucleotide or the polypeptide encoded within the polynucleotide. The experiment preferably includes a control sample which does not receive the candidate agent. The treated and untreated cells are then compared by any suitable phenotypic criteria, including but not limited to microscopic analysis, viability testing, ability to replicate, histological examination, the level of a particular RNA or polypeptide associated with the cells, the level of enzymatic activity expressed by the cells or cell lysates, the interactions of the cells when exposed to infectious agents, such as HIV, and the ability of the cells to interact with other cells or compounds. For example, the transfected cells can be exposed to the agent to be tested and, before, during, or after treatment with the agent, the cells can be infected with a virus, such as HCV or HIV, and tested for any indication of susceptibility of the cells to viral infection, including, for example, susceptibility of the cells to cell-to-cell viral infection, replication of the virus, production of a viral protein, and/or syncytia formation following infection with the virus. Differences between treated and untreated cells indicate effects attributable to the candidate agent. Optimally, the agent has a greater effect on experimental cells than on control cells. Appropriate host cells include, but are not limited to, eukaryotic cells, preferably mammalian cells. The choice of cell will at least partially depend on the nature of the assay contemplated.

To test for agents that upregulate the expression of a polynucleotide, a suitable host cell transfected with a polynucleotide of interest, such that the polynucleotide is expressed (as used herein, expression includes transcription and/or translation) is contacted with an agent to be tested. An agent would be tested for its ability to result in increased expression of mRNA and/or polypeptide. Methods of making vectors and transfection are well known in the art. “Transfection” encompasses any method of introducing the exogenous sequence, including, for example, lipofection, transduction, infection or electroporation. The exogenous polynucleotide may be maintained as a non-integrated vector (such as a plasmid) or may be integrated into the host genome.

To identify agents that specifically activate transcription, transcription regulatory regions could be linked to a reporter gene and the construct added to an appropriate host cell. As used herein, the term “reporter gene” means a gene that encodes a gene product that can be identified (i.e., a reporter protein). Reporter genes include, but are not limited to, alkaline phosphatase, chloramphenicol acetyltransferase, β-galactosidase, luciferase and green fluorescence protein (GFP). Identification methods for the products of reporter genes include, but are not limited to, enzymatic assays and fluorimetric assays. Reporter genes and assays to detect their products are well known in the art and are described, for example in Ausubel et al. (1987) and periodic updates. Reporter genes, reporter gene assays, and reagent kits are also readily available from commercial sources. Examples of appropriate cells include, but are not limited to, fungal, yeast, mammalian, and other eukaryotic cells. A practitioner of ordinary skill will be well acquainted with techniques for transfecting eukaryotic cells, including the preparation of a suitable vector, such as a viral vector; conveying the vector into the cell, such as by electroporation; and selecting cells that have been transformed, such as by using a reporter or drug sensitivity element. The effect of an agent on transcription from the regulatory region in these constructs would be assessed through the activity of the reporter gene product.

Besides the increase in expression under conditions in which it is normally repressed mentioned above, expression could be decreased when it would normally be maintained or increased. An agent could accomplish this through a decrease in transcription rate and the reporter gene system described above would be a means to assay for this. The host cells to assess such agents would need to be permissive for expression.

Cells transcribing mRNA (from the polynucleotide of interest) could be used to identify agents that specifically modulate the half-life of mRNA and/or the translation of mRNA. Such cells would also be used to assess the effect of an agent on the processing and/or post-translational modification of the polypeptide. An agent could modulate the amount of polypeptide in a cell by modifying the turnover (i.e., increase or decrease the half-life) of the polypeptide. The specificity of the agent with regard to the mRNA and polypeptide would be determined by examining the products in the absence of the agent and by examining the products of unrelated mRNAs and polypeptides. Methods to examine mRNA half-life, protein processing, and protein turn-over are well know to those skilled in the art.

In vivo screening methods could also be useful in the identification of agents that modulate polypeptide function through the interaction with the polypeptide directly. Such agents could block normal polypeptide-ligand interactions, if any, or could enhance or stabilize such interactions. Such agents could also alter a conformation of the polypeptide. The effect of the agent could be determined using immunoprecipitation reactions. Appropriate antibodies would be used to precipitate the polypeptide and any protein tightly associated with it. By comparing the polypeptides immunoprecipitated from treated cells and from untreated cells, an agent could be identified that would augment or inhibit polypeptide-ligand interactions, if any. Polypeptide-ligand interactions could also be assessed using cross-linking reagents that convert a close, but noncovalent interaction between polypeptides into a covalent interaction. Techniques to examine protein-protein interactions are well known to those skilled in the art. Techniques to assess protein conformation are also well known to those skilled in the art.

It is also understood that screening methods can involve in vitro methods, such as cell-free transcription or translation systems. In those systems, transcription or translation is allowed to occur, and an agent is tested for its ability to modulate function. For an assay that determines whether an agent modulates the translation of mRNA or a polynucleotide, an in vitro transcription/translation system may be used. These systems are available commercially and provide an in vitro means to produce mRNA corresponding to a polynucleotide sequence of interest. After mRNA is made, it can be translated in vitro and the translation products compared. Comparison of translation products between an in vitro expression system that does not contain any agent (negative control) with an in vitro expression system that does contain an agent indicates whether the agent is affecting translation. Comparison of translation products between control and test polynucleotides indicates whether the agent, if acting on this level, is selectively affecting translation (as opposed to affecting translation in a general, non-selective or non-specific fashion). The modulation of polypeptide function can be accomplished in many ways including, but not limited to, the in vivo and in vitro assays listed above as well as in in vitro assays using protein preparations. Polypeptides can be extracted and/or purified from natural or recombinant sources to create protein preparations. An agent can be added to a sample of a protein preparation and the effect monitored; that is whether and how the agent acts on a polypeptide and affects its conformation, folding (or other physical characteristics), binding to other moieties (such as ligands), activity (or other functional characteristics), and/or other aspects of protein structure or functions is considered to have modulated polypeptide function.

In an example for an assay for an agent that binds to a polypeptide encoded by a polynucleotide identified by the methods described herein, a polypeptide is first recombinantly expressed in a prokaryotic or eukaryotic expression system as a native or as a fusion protein in which a polypeptide (encoded by a polynucleotide identified as described above) is conjugated with a well-characterized epitope or protein. Recombinant polypeptide is then purified by, for instance, immunoprecipitation using appropriate antibodies or anti-epitope antibodies or by binding to immobilized ligand of the conjugate. An affinity column made of polypeptide or fusion protein is then used to screen a mixture of compounds which have been appropriately labeled. Suitable labels include, but are not limited to fluorochromes, radioisotopes, enzymes and chemiluminescent compounds. The unbound and bound compounds can be separated by washes using various conditions (e.g. high salt, detergent) that are routinely employed by those skilled in the art. Non-specific binding to the affinity column can be minimized by pre-clearing the compound mixture using an affinity column containing merely the conjugate or the epitope. Similar methods can be used for screening for an agent(s) that competes for binding to polypeptides. In addition to affinity chromatography, there are other techniques such as measuring the change of melting temperature or the fluorescence anisotropy of a protein which will change upon binding another molecule. For example, a BIAcore assay using a sensor chip (supplied by Pharmacia Biosensor, Stitt et al. (1995) Cell 80: 661-670) that is covalently coupled to polypeptide may be performed to determine the binding activity of different agents.

It is also understood that the in vitro screening methods of this invention include structural, or rational, drug design, in which the amino acid sequence, three-dimensional atomic structure or other property (or properties) of a polypeptide provides a basis for designing an agent which is expected to bind to a polypeptide. Generally, the design and/or choice of agents in this context is governed by several parameters, such as side-by-side comparison of the structures of a human and homologous non-human primate polypeptides, the perceived function of the polypeptide target, its three-dimensional structure (if known or surmised), and other aspects of rational drug design. Techniques of combinatorial chemistry can also be used to generate numerous permutations of candidate agents.

Also contemplated in screening methods of the invention are transgenic animal systems and animal models containing chimeric organs or tissues, which are known in the art.

The screening methods described above represent primary screens, designed to detect any agent that may exhibit activity that modulates the function of a polynucleotide or polypeptide. The skilled artisan will recognize that secondary tests will likely be necessary in order to evaluate an agent further. For example, a secondary screen may comprise testing the agent(s) in an infectivity assay using mice and other animal models (such as rat), which are known in the art. In addition, a cytotoxicity assay would be performed as a further corroboration that an agent which tested positive in a primary screen would be suitable for use in living organisms. Any assay for cytotoxicity would be suitable for this purpose, including, for example the MTT assay (Promega).

The invention also includes agents identified by the screening methods described herein.

### Methods Useful for Identifying Positively Selected Non-Human Traits

In one aspect of the invention, a non-human primate polynucleotide or polypeptide has undergone natural selection that resulted in a positive evolutionarily significant change (i.e., the non-human primate polynucleotide or polypeptide has a positive attribute not present in humans). In this aspect of the invention, the positively selected polynucleotide or polypeptide may be associated with susceptibility or resistance to certain diseases or with other commercially relevant traits. Examples of this embodiment include, but are not limited to, polynucleotides and polypeptides that have been positively selected in non-human primates, preferably chimpanzees, that may be associated with susceptibility or resistance to infectious diseases, cancer, or acne or may be associated with aesthetic conditions of interest to humans, such as hair growth or muscle mass. An example of this embodiment includes polynucleotides and polypeptides associated with the susceptibility or resistance to HIV progression to AIDS. The present invention can thus be useful in gaining insight into the molecular mechanisms that underlie resistance to HIV infection progressing to development of AIDS, providing information that can also be useful in discovering and/or designing agents such as drugs that prevent and/or delay development of AIDS. For example, CD59, which has been identified as a leukocyte and erythrocyte protein whose function is to protect these cells from the complement arm of the body=s MAC (membrane attack complex) defense system (Meri et al. (1996) Biochem. J. 616:923-935), has been found to be positively selected in the chimpanzee (see Example 16). It is believed that the CD59 found in chimpanzees confers a resistance to the progression of AIDS that is not found in humans. Thus, the positively selected chimpanzee CD59 can serve in the development of agents or drugs that are useful in arresting the progression of AIDS in humans, as is described in the Examples.

Another example involves the p44 polynucleotides and polypeptides associated with resistance to HCV infection in chimpanzees. This discovery can be useful in discerning the molecular mechanisms that underlie resistance to HCV infection progression to chronic hepatitis and/or hepatocellular carcinoma in chimpanzees, and in providing information useful in the discovery and/or design of agents that prevent and/or delay chronic hepatitis or hepatocellular carcinoma.

Commercially relevant examples include, but are not limited to, polynucleotides and polypeptides that are positively selected in non-human primates that may be associated with aesthetic traits, such as hair growth, acne, or muscle mass. Accordingly, in one aspect, the invention provides methods for identifying a polynucleotide sequence encoding a polypeptide, wherein said polypeptide may be associated with a medically or commercially relevant positive evolutionarily significant change. The method comprises the steps of: (a) comparing human protein-coding nucleotide sequences to protein-coding nucleotide sequences of a non-human primate; and (b) selecting a non-human primate polynucleotide sequence that contains at least one nucleotide change as compared to corresponding sequence of the human, wherein said change is evolutionarily significant. The sequences identified by this method may be further characterized and/or analyzed for their possible association with biologically or medically relevant functions unique or enhanced in non-human primates.

### Methods Useful for Identifying Positively Selected Human Traits

This invention specifically provides methods for identifying human polynucleotide and polypeptide sequences that may be associated with unique or enhanced functional capabilities or traits of the human, for example, brain function or longer life span. More particularly, these methods identify those genetic sequences that may be associated with capabilities that are unique or enhanced in humans, including, but not limited to, brain functions such as high capacity information processing, storage and retrieval capabilities, creativity, and language abilities. Moreover, these methods identify those sequences that may be associated to other brain functional features with respect to which the human brain performs at enhanced levels as compared to other non-human primates; these differences may include brain-mediated emotional response, locomotion, pain/pleasure sensation, olfaction, temperament and longer life span.

In this method, the general methods of the invention are applied as described above. Generally, the methods described herein entail (a) comparing human protein-coding polynucleotide sequences to that of a non-human primate; and (b) selecting those human protein-coding polynucleotide sequences having evolutionarily significant changes that may be associated with unique or enhanced functional capabilities of the human as compared to that of the non-human primate.

In this embodiment, the human sequence includes the evolutionarily significant change (i.e., the human sequence differs from more than one non-human primate species sequence in a manner that suggests that such a change is in response to a selective pressure). The identity and function of the protein encoded by the gene that contains the evolutionarily significant change is characterized and a determination is made whether or not the protein can be involved in a unique or enhanced human function. If the protein is involved in a unique or enhanced human function, the information is used in a manner to identify agents that can supplement or otherwise modulate the unique or enhanced human function.

As a non-limiting example of the invention, identifying the genetic (i.e., nucleotide sequence) differences underlying the functional uniqueness of human brain may provide a basis for designing agents that can modulate human brain functions and/or help correct functional defects. These sequences could also be used in developing diagnostic reagents and/or biomedical research tools. The invention also provides methods for a large-scale comparison of human brain protein-coding sequences with those from a non-human primate.

The identified human sequence changes can be used in establishing a database of candidate human genes that may be involved in human brain function. Candidates are ranked as to the likelihood that the gene is responsible for the unique or enhanced functional capabilities found in the human brain compared to chimpanzee or other non-human primates. Moreover, the database not only provides an ordered collection of candidate genes, it also provides the precise molecular sequence differences that exist between human and chimpanzee (and other non-human primates), and thus defines the changes that underlie the functional differences. This information can be useful in the identification of potential sites on the protein that may serve as useful targets for pharmaceutical agents.

Accordingly, the present invention also provides methods for correlating an evolutionarily significant nucleotide change to a brain functional capability that is unique or enhanced in humans, comprising (a) identifying a human nucleotide sequence according to the methods described above; and (b) analyzing the functional effect of the presence or absence of the identified sequence in a model system.

Further studies can be carried out to confirm putative function. For example, the putative function can be assayed in appropriate in vitro assays using transiently or stably transfected mammalian cells in culture, or using mammalian cells transfected with an antisense clone to inhibit expression of the identified polynucleotide to assess the effect of the absence of expression of its encoded polypeptide. Studies such as one-hybrid and two-hybrid studies can be conducted to determine, for example, what other macromolecules the polypeptide interacts with. Transgenic nematodes or Drosophila can be used for various functional assays, including behavioral studies. The appropriate studies depend on the nature of the identified polynucleotide and the polypeptide encoded within the polynucleotide, and would be obvious to those skilled in the art.

The present invention also provides polynucleotides and polypeptides identified by the methods of the present invention. In one embodiment, the present invention provides an isolated AATYK nucleotide sequence selected from the group consisting of nucleotides 2180-2329 of SEQ ID NO:14, nucleotides 2978-3478 of SEQ ID NO:14, and nucleotides 3380-3988 of SEQ ID NO:14; and an isolated nucleotide sequence having at least 85% homology to a nucleotide sequence of any of the preceding sequences.

In another embodiment, the invention provides an isolated AATYK polypeptide selected from the group consisting of a polypeptide encoded by a nucleotide sequence selected from the group consisting of SEQ ID NO:17 and SEQ ID NO:18; wherein said encoding is based on the open reading frame (ORF) of SEQ ID NO:14, and a polypeptide encoded by a nucleotide sequence having at least 85% homology to a nucleotide sequence selected from the group consisting of SEQ ID NO:17 and SEQ ID NO:18; wherein said encoding is based on the open reading frame of SEQ ID NO:14.

In a further embodiment, the present invention provides an isolated AATYK polypeptide selected from the group consisting of a polypeptide encoded by a nucleotide sequence selected from the group consisting of nucleotides 1-501 of SEQ ID NO:17, nucleotides 1-150 of SEQ ID NO:17, nucleotides 100-249 of SEQ ID NO:17, nucleotides 202-351 of SEQ ID NO:17, nucleotides 301-450 of SEQ ID NO:17, nucleotides 799-948 of SEQ ID NO:17, nucleotides 901-1050 of SEQ ID NO:17, nucleotides 799-1299 of SEQ ID NO:17, and nucleotides 1201-1809 of SEQ ID NO:17; wherein said encoding is based on the open reading frame of SEQ ID NO:14; and a polypeptide encoded by a nucleotide sequence having at least 85% homology to any of the preceding nucleotide sequences.

In still another embodiment, the invention provides an isolated polypeptide selected from the group consisting of a polypeptide encoded by a nucleotide sequence selected from the group consisting of nucleotides 1-501 of SEQ ID NO:18, nucleotides 799-1299 of SEQ ID NO:18, and nucleotides 1201-1809 of SEQ ID NO:18; wherein said encoding is based on the open reading frame of SEQ ID NO:14; and a polypeptide encoded by a nucleotide sequence having at least 85% homology to nucleotides 1-501 of SEQ ID NO:18, nucleotides 799-1299 of SEQ ID NO:18, and nucleotides 1201-1809 of SEQ ID NO:18.

In another embodiment, the invention provides an isolated polynucleotide comprising SEQ ID NO:17, wherein the coding capacity of the nucleic acid molecule is based on the open reading frame of SEQ ID NO:14. In a preferred embodiment, the polynucleotide is a Pan troglodytes polynucleotide.

In another embodiment, the invention provides an isolated polynucleotide comprising SEQ ID NO:18, wherein the coding capacity of the nucleic acid molecule is based on the open reading frame of SEQ ID NO:14. In a preferred embodiment, the polynucleotide is a Gorilla gorilla polynucleotide.

In some embodiments, the polynucleotide or polypeptide having 85% homology to an isolated AATYK polynucleotide or polypeptide of the present invention is a homolog, which, when compared to a non-human primate, yields a K_{A}/K_{S} ratio of at least 0.75, at least 1.00, at least 1.25, at least 1.50, or at least 2.00.

In other embodiments, the polynucleotide or polypeptide having 85% homology to an isolated AATYK polynucleotide or polypeptide of the present invention is a homolog which is capable of performing the function of the natural AATYK polynucleotide or polypeptide in a functional assay. Suitable assays for assessing the function of an ATTYK polynucleotide or polypeptide include a neuronal differentiation assay such as that described by Raghunath, et al., Brain Res Mol Brain Res. (2000) 77:151-62, or a tyrosine phosphorylation assay such as that described in Tomomura, et al., Oncogene (2001) 20(9):1022-32. The phrase “capable of performing the function of the natural AATYK polynucleotide or polypeptide in a functional assay” means that the polynucleotide or polypeptide has at least about 10% of the activity of the natural polynucleotide or polypeptide in the functional assay. In other preferred embodiments, has at least about 20% of the activity of the natural polynucleotide or polypeptide in the functional assay. In other preferred embodiments, has at least about 30% of the activity of the natural polynucleotide or polypeptide in the functional assay. In other preferred embodiments, has at least about 40% of the activity of the natural polynucleotide or polypeptide in the functional assay. In other preferred embodiments, has at least about 50% of the activity of the natural polynucleotide or polypeptide in the functional assay. In other preferred embodiments, the polynucleotide or polypeptide has at least about 60% of the activity of the natural polynucleotide or polypeptide in the functional assay. In more preferred embodiments, the polynucleotide or polypeptide has at least about 70% of the activity of the natural polynucleotide or polypeptide in the functional assay. In more preferred embodiments, the polynucleotide or polypeptide has at least about 80% of the activity of the natural polynucleotide or polypeptide in the functional assay. In more preferred embodiments, the polynucleotide or polypeptide has at least about 90% of the activity of the natural polynucleotide or polypeptide in the functional assay.

### Description of the AIDS Embodiment (an Example of a Positively Selected Non-Human Trait)

The AIDS (Acquired Immune Deficiency Syndrome) epidemic has been estimated to threaten 30 million people world-wide (UNAIDS/WHO, 1998, “Report on the global HIV/AIDS epidemic”). Well over a million people are infected in developed countries, and in parts of sub-Saharan Africa, 1 in 4 adults now carries the virus (UNAIDS/WHO, 1998). Although efforts to develop vaccines are underway, near term prospects for successful vaccines are grim. Balter and Cohen (1998) Science 281:159-160; Baltimore and Heilman (1998) Scientific Am. 279:98-103. Further complicating the development of therapeutics is the rapid mutation rate of HIV (the human immunodeficiency virus which is responsible for AIDS), which generates rapid changes in viral proteins. These changes ultimately allow the virus to escape current therapies, which target viral proteins. Dobkin (1998) Inf. Med. 15(3): 159. Even drug cocktails which initially showed great promise are subject to the emergence of drug-resistant mutants. Balter and Cohen (1998); Dobkin (1998). Thus, there is still a serious need for development of therapies which delay or prevent progression of AIDS in HIV-infected individuals. Chun et al. (1997) Proc. Natl. Acad. Sci. USA 94:13193-13197; Dobkin (1998).

Human=s closest relatives, chimpanzees (Pan troglodytes), have unexpectedly proven to be poor models for the study of the disease processes following infection with HIV-1. Novembre et al. (1997); J. Virol. 71(5):4086-4091. Once infected with HIV-1, chimpanzees display resistance to progression of the disease. To date, only one chimpanzee individual is known to have developed full-blown AIDS, although more than 100 captive chimpanzees have been infected. Novembre et al. (1997); Villinger et al. (1997) J. Med. Primatol. 26(1-2): 11-18. Clearly, an understanding of the mechanism(s) that confer resistance to progression of the disease in chimpanzees may prove invaluable for efforts to develop therapeutic agents for HIV-infected humans.

It is generally believed that wild chimpanzee populations harbored the HIV-1 virus (perhaps for millennia) prior to its recent cross-species transmission to humans. Dube et al, (1994); Virology 202:379-389; Zhu and Ho (1995) Nature 374:503-504; Zhu et al. (1998); Quinn (1994) Proc. Natl. Acad. Sci. USA 91:2407-2414. During this extended period, viral/host co-evolution has apparently resulted in accommodation, explaining chimpanzee resistance to AIDS progression. Burnet and White (1972); Natural History of Infectious Disease (Cambridge, Cambridge Univ. Press); Ewald (1991) Hum. Nat. 2(i):1-30. All references cited herein are hereby incorporated by reference in their entirety.

One aspect of this invention arises from the observations that (a) because chimpanzees (Pan troglodytes) have displayed resistance to development of AIDS although susceptible to HIV infection (Alter et al. (1984) Science 226:549-552; Fultz et al. (1986) J. Virol. 58:116-124; Novembre et al. (1997) J. Virol. 71(5):4086-4091), while humans are susceptible to developing this devastating disease, certain genes in chimpanzees may contribute to this resistance; and (b) it is possible to evaluate whether changes in human genes when compared to homologous genes from other species (such as chimpanzee) are evolutionarily significant (i.e., indicating positive selective pressure). Thus, protein coding polynucleotides may contain sequence changes that are found in chimpanzees (as well as other AIDS-resistant primates) but not in humans, likely as a result of positive adaptive selection during evolution. Furthermore, such evolutionarily significant changes in polynucleotide and polypeptide sequences may be attributed to an AIDS-resistant non-human primate=s (such as chimpanzee) ability to resist development of AIDS. The methods of this invention employ selective comparative analysis to identify candidate genes which may be associated with susceptibility or resistance to AIDS, which may provide new host targets for therapeutic intervention as well as specific information on the changes that evolved to confer resistance. Development of therapeutic approaches that involve host proteins (as opposed to viral proteins and/or mechanisms) may delay or even avoid the emergence of resistant viral mutants. The invention also provides screening methods using the sequences and structural differences identified.

This invention provides methods for identifying human polynucleotide and polypeptide sequences that may be associated with susceptibility to post-infection development of AIDS. Conversely, the invention also provides methods for identifying polynucleotide and polypeptide sequences from an AIDS-resistant non-human primate (such as chimpanzee) that may be associated with resistance to development of AIDS. Identifying the genetic (i.e., nucleotide sequence) and the resulting protein structural and biochemical differences underlying susceptibility or resistance to development of AIDS will likely provide a basis for discovering and/or designing agents that can provide prevention and/or therapy for HIV infection progressing to AIDS. These differences could also be used in developing diagnostic reagents and/or biomedical research tools. For example, identification of proteins which confer resistance may allow development of diagnostic reagents or biomedical research tools based upon the disruption of the disease pathway of which the resistant protein plays a part.

Generally, the methods described herein entail (a) comparing human protein-coding polynucleotide sequences to that of an AIDS resistant non-human primate (such as chimpanzee), wherein the human protein coding polynucleotide sequence is associated with development of AIDS; and (b) selecting those human protein-coding polynucleotide sequences having evolutionarily significant changes that may be associated with susceptibility to development of AIDS. In another embodiment, the methods entail (a) comparing human protein-coding polynucleotide sequences to that of an AIDS-resistant non-human primate (such as chimpanzee), wherein the human protein coding polynucleotide sequence is associated with development of AIDS; and (b) selecting those non-human primate protein-coding polynucleotide sequences having evolutionarily significant changes that may be associated with resistance to development of AIDS.

As is evident, the methods described herein can be applied to other infectious diseases. For example, the methods could be used in a situation in which a non-human primate is known or believed to have harbored the infectious disease for a significant period (i.e., a sufficient time to have allowed positive selection) and is resistant to development of the disease. Thus, in other embodiments, the invention provides methods for identifying a polynucleotide sequence encoding a polypeptide, wherein said polypeptide may be associated with resistance to development of an infectious disease, comprising the steps of: (a) comparing infectious disease-resistant non-human primate protein coding sequences to human protein coding sequences, wherein the human protein coding sequence is associated with development of the infectious disease; and (b) selecting an infectious disease-resistant non-human primate sequence that contains at least one nucleotide change as compared to the corresponding human sequence, wherein the nucleotide change is evolutionarily significant. In another embodiment, the invention provides methods for identifying a human polynucleotide sequence encoding a polypeptide, wherein said polypeptide may be associated with susceptibility to development of an infectious disease, comprising the steps of: (a) comparing human protein coding sequences to protein-coding polynucleotide sequences of an infectious disease-resistant non-human primate, wherein the human protein coding sequence is associated with development of the infectious disease; and (b) selecting a human polynucleotide sequence that contains at least one nucleotide change as compared to the corresponding sequence of an infectious disease-resistant non-human primate, wherein the nucleotide change is evolutionarily significant.

In the present invention, human sequences to be compared with a homologue from an AIDS-resistant non-human primate are selected based on their known or implicated association with HIV propagation (i.e., replication), dissemination and/or subsequent progression to AIDS. Such knowledge is obtained, for example, from published literature and/or public databases (including sequence databases such as GenBank). Because the pathway involved in development of AIDS (including viral replication) involves many genes, a number of suitable candidates may be tested using the methods of this invention. Table 1 contains a exemplary list of genes to be examined. The sequences are generally known in the art.

TABLE 1
Sample List of Human Genes to be/have been Examined

| Gene | Function |
|---|---|
| eIF-5A | initiation factor |
| hPC6A | protease |
| hPC6B | protease |
| P56^{lck} | Signal transduction |
| FK506-binding protein | Immunophilin |
| calnexin | ? |
| Bax | PCD promoter |
| bcl-2 | apoptosis inhibitor |
| lck | tyrosine kinase |
| MAPK (mitogen activated protein kinase) | protein kinase |
| CD43 | sialoglycoprotein |
| CCR2B | chemokine receptor |
| CCR3 | chemokine receptor |
| Bonzo | chemokine receptor |
| BOB | chemokine receptor |
| GPR1 | chemokine receptor |
| stromal-derived factor-1 (SDF-1) | chemokine |
| tumor-necrosis factor-α (TNF- α) | PCD promoter |
| TNF-receptor II (TNFRII) | receptor |
| interferon γ (IFN- γ) | cytokine |
| interleukin 1 α(IL-1 α) | cytokine |
| interleukin 1β(IL-1 β) | cytokine |
| interleukin 2 (IL-2) | cytokine |
| interleukin 4 (IL-4) | cytokine |
| interleukin 6 (IL-6) | cytokine |
| interleukin 10 (IL-10) | cytokine |
| interleukin 13 (IL-13) | cytokine |
| B7 | signaling protein |
| macrophage colony-st imulating factor (M-CSF) | cytokine |
| granulocyte-macrophage colony-stimulating factor | cytokine |
| phosphatidylinositol 3-kinase (PI 3-kinase) | kinase |
| phosphatidylinositol 4-kinase (PI 4-kinase) | kinase |
| HLA class I α chain | histocompatibility |
| | antigen |
| β₂ microglobulin | lymphocyte antigen |
| CD55 | decay-accelerating |
| | factor |
| CD63 | glycoprotein antigen |
| CD71 | ? |
| interferon α (IFN- α) | cytokine |
| CD44 | cell adhesion |
| CD8 | glycoprotein |

Genes already examined (13)

| | |
|---|---|
| ICAM-1 | Immune system |
| ICAM-2 | Immune system |
| ICAM-3 | Immune system |
| leukocyte associated function 1 molecule α | Immune system |
| (LFA-1) | |
| leukocyte associated function 1 molecule β | Immune system |
| (LFA-1) | |
| Mac-1 α | Immune system |
| Mac-1 β (equivalent to LFA-1β) | Immune system |
| DC-SIGN | Immune system |
| CD59 | complement protein |
| CXCR4 | chemokine receptor |
| CCR5 | chemokine receptor |
| MIP-1α | chemokine |
| MIP-1β | chemokine |
| RANTES | chemokine |

Aligned protein-coding sequences of human and an AIDS resistant non-human primate such as chimpanzee are analyzed to identify nucleotide sequence differences at particular sites. The detected sequence changes are generally, and preferably, initially checked for accuracy as described above. The evolutionarily significant nucleotide changes, which are detected by molecular evolution analysis such as the K_{A}/K_{S} analysis, can be further assessed to determine whether the non-human primate gene or the human gene has been subjected to positive selection. For example, the identified changes can be tested for presence/absence in other AIDS-resistant non-human primate sequences. The sequences with at least one evolutionarily significant change between human and one AIDS-resistant non-human primate can be used as primers for PCR analysis of other non-human primate protein-coding sequences, and resulting polynucleotides are sequenced to see whether the same change is present in other non-human primates. These comparisons allow further discrimination as to whether the adaptive evolutionary changes are unique to the AIDS-resistant non-human primate (such as chimpanzee) as compared to other non-human primates. For example, a nucleotide change that is detected in chimpanzee but not other primates more likely represents positive selection on the chimpanzee gene. Other non-human primates used for comparison can be selected based on their phylogenetic relationships with human. Closely related primates can be those within the hominoid sublineage, such as chimpanzee, bonobo, gorilla, and orangutan. Non-human primates can also be those that are outside the hominoid group and thus not so closely related to human, such as the Old World monkeys and New World monkeys. Statistical significance of such comparisons may be determined using established available programs, e.g., t-test as used by Messier and Stewart (1997) Nature 385:151-154.

Furthermore, sequences with significant changes can be used as probes in genomes from different humans to see whether the sequence changes are shared by more than one individual. For example, certain individuals are slower to progress to AIDS (“slow progressers”) and comparison (a) between a chimpanzee sequence and the homologous sequence from the slow-progresser human individual and/or (b) between an AIDS-susceptible individual and a slow-progresser individual would be of interest. Gene sequences from different human populations can be obtained from databases made available by, for example, the human genome diversity project or, alternatively, from direct sequencing of PCR-amplified DNA from a number of unrelated, diverse human populations. The presence of the identified changes in human slow progressers would further indicate the evolutionary significance of the changes.

As is exemplified herein, the CD59 protein, which has been associated with the chimpanzee=s resistance to the progression of AIDS, exhibits an evolutionarily significant nucleotide change relative to human CD59. CD59 (also known as protectin, 1F-5Ag, H19, HRF20, MACIF, MIRL and P-18) is expressed on peripheral blood leukocytes and erythrocytes, and functions to restrict lysis of human cells by complement (Meri et al (1996) Biochem. J. 316:923). More specifically, CD59 acts as an inhibitor of membrane attack complexes, which are complement proteins that make hole-like lesions in the cell membranes. Thus, CD59 protects the cells of the body from the complement arm of its own defense system (Meri et al, supra). The chimpanzee homolog of this protein was examined because the human homolog has been implicated in the progression of AIDS in infected individuals. It has been shown that CD59 is one of the host cell derived proteins that is selectively taken up by HIV virions (Frank et al. (1996) AIDS 10:1611). Additionally, it has been shown that HIV virions that have incorporated host cell CD59 are protected from the action of complement. Thus, in humans, HIV uses CD59 to protect itself from attack by the victim=s immune system, and thus to further the course of infection. As is theorized in the examples, positively-selected chimpanzee CD59 may constitute the adaptive change that inhibits disease progression. The virus may be unable to usurp the chimpanzee=s CD59 protective role, thereby rendering the virus susceptible to the chimpanzee=s immune system.

As is further exemplified herein, the DC-SIGN protein has also been determined to be positively selected in the chimpanzee as compared to humans and gorilla. DC-SIGN is expressed on dendritic cells and has been documented to provide a mechanism for travel of the HIV-1 virus to the lymph nodes where it infects undifferentiated T cells (Geijtenbeek, T. B. H. et al. (2000) Cell 100:587-597). Infection of the T cells ultimately leads to compromise of the immune system and subsequently to full-blown AIDS. The HIV-1 virus binds to the extracellular portion of DC-SIGN, and then gains access to the T cells via their CD4 proteins. DC-SIGN has as its ligand ICAM-3, which has a very high K_{A}/K_{S} ratio. It may be that the positive selection on chimpanzee ICAM-3 was a result of compensatory changes to permit continued binding to DC-SIGN. As is theorized in the examples, positively-selected chimpanzee DC-SIGN may constitute another adaptive change that inhibits disease progression. Upon resolution of the three-dimensional structure of chimpanzee DC-SIGN and identification of the mechanism by which HIV-1 is prevented from binding to DC-SIGN, it may be possible to design drugs to mimic the effects of chimpanzee DC-SIGN without disrupting the normal functions of human DC-SIGN.

In one embodiment, the present invention includes a method to identify an agent which may modulate resistance to HIV-1 mediated disease, comprising contacting at least one agent to be tested with a cell comprising human ICAM-1, and detecting the cell's resistance to HIV-1 viral replication, propagation, or function, wherein an agent is identified by its ability to increase the cell's resistance to HIV-1 viral replication, propagation, or function. In other embodiments, the disease may be an RNA virus-mediated disease and/or an HCV-virus mediated disease. Methods to detect RNA virus and/or HCV-virus replication, propagation, or function are routinely known in the art and are detailed herein.

In one embodiment of the instant method, increased resistance to RNA virus, HCV virus, HIV-1 viral replication, propagation, or function is measured relative to that of a cell transfected with an effective amount of at least one of the following: a mutant human ICAM-1 comprising one or more of the following mutations to human ICAM-1:

L18Q, K29D, P45G, R49W, E171Q, wherein the mutant ICAM-1 is otherwise identical to human ICAM-1; and a primate ICAM-1. In one embodiment, the human ICAM-1 sequence is SEQ ID NO:3. In one particular embodiment of the instant invention, wherein the primate ICAM-1 is a chimpanzee ICAM-1 comprising SEQ ID NO:85. In another embodiment of the instant invention, the resistance to viral replication or propagation is demonstrated by reduction of RNA virus, HCV virus, HIV-1 expression in RNA virus, HCV virus, HIV-1 infected cells. In another embodiment of the instant invention, the resistance to viral replication or propagation is a result of increased dimerization of two ICAM-1 polypeptides in the cell. In yet another embodiment of the instant invention, the resistance to viral replication or propagation is a result of decreased dimerization of two ICAM-1 polypeptides in the cell.

In all inventive compositions and inventions, resistance to viral replication, propagation, or function may be determined by measurement of virus-mediated cellular pathogenesis, cell to cell infectivity, virus-mediated cell fusion, virus-mediated syncytia formation, HIV-1 expression by the cell, inflammatory response suppression, and virus budding rate, among other methods known in the art. In one embodiment, an agent is a small molecule.

In another embodiment, the present invention includes a human mutant ICAM-1 polypeptide comprising one or more of the following mutations to human ICAM-1: L18Q, K29D, P45G, R49W, E171Q, wherein the mutant ICAM-1 is otherwise identical to human ICAM-1, wherein said polypeptide confers increased resistance to RNA virus, HCV virus, HIV-1 viral replication, propagation, or function in a human cell.

In another embodiment, the present invention includes a human cell comprising heterologous DNA encoding a mutant human ICAM-1 comprising one or more of the following mutations to human ICAM-1: L18Q, K29D, P45G, R49W, E171Q wherein the mutant ICAM-1 is otherwise identical to human ICAM-1 and wherein said polypeptide confers increased resistance to HIV-1 viral replication, propagation, or function in a human cell; and a primate ICAM-1. In one embodiment, the primate ICAM-1 is a chimpanzee ICAM-1.

In another embodiment, the present invention includes a method for inhibiting RNA virus, HCV virus, HIV-1 viral replication, propagation, or function in a human subject by ICAM-1 gene therapy, comprising the steps of: parenterally administering to a human subject at least one of the following: a viral vector comprising a mutant ICAM-1 comprising one or more of the following mutations: L18Q, K29D, P45G, R49W, E171Q, and a viral vector comprising a non-human primate ICAM-1, allowing said ICAM-1 protein to be expressed from said gene in said subject in an amount sufficient to provide for inhibiting HIV-1 viral replication, propagation, or function in the human subject. In one embodiment, increased resistance to AIDS comprises inhibition of production of HIV-1 in the subject. In one embodiment, the primate ICAM-1 is a chimpanzee ICAM-1. In another embodiment, the present invention includes a method for inhibiting RNA virus, HCV virus, HIV-1 viral replication, propagation, or function in a human subject by ICAM-1 gene therapy, comprising the steps of: transfection of at least a portion of the subject's white blood cells with at least one of the following: a viral vector comprising a mutant ICAM-1 comprising one or more of the following mutations: L18Q, K29D, P45G, R49W, E1171Q, and a viral vector comprising a non-human primate ICAM-1, allowing said ICAM-1 protein to be expressed from at least a portion of the transfected white blood cells, in an amount sufficient to provide for inhibiting HIV-1 viral replication, propagation, or function in the human subject. In one embodiment, the primate ICAM-1 is a chimpanzee ICAM-1. In one embodiment of the methods, at least a portion of the subject's white blood cells are removed from the subject prior to transfection and returned to the subject post-transfection.

The present invention also includes a method to treat an RNA virus, HCV virus, HIV-1 infection in a human subject, comprising administering a pharmaceutically effective amount of an agent which increases the human subject's resistance to RNA virus, HCV virus, HIV-1 viral replication, propagation, or function by modulating the function of human ICAM-1. In one embodiment, the modulation of the function of human ICAM-1 results in resistance to RNA virus, HCV virus, HIV-1 viral replication, propagation, or function that is substantially similar to that provided by at least one of the following: a mutant human ICAM-1 comprising one or more of the following mutations to human ICAM-1: L18Q, K29D, P45G, R49W, E171Q wherein the mutant ICAM-1 is otherwise identical to human ICAM-1; and a primate ICAM-1. In one embodiment, the resistance to viral replication or propagation is reduction of RNA virus, HCV virus, HIV-1 expression in RNA virus, HCV virus, HIV-1 infected cells. In one embodiment, the resistance to viral replication or propagation is a result of increased dimerization of two ICAM-1 polypeptides. In another embodiment, the resistance to viral replication or propagation is a result of decreased dimerization of two ICAM-1 polypeptides. In one embodiment, resistance to viral replication, propagation, or function is determined by measurement of virus-mediated cellular pathogenesis, cell to cell infectivity, virus-mediated cell fusion, virus-mediated syncytia formation, RNA virus, HCV virus, HIV-1 expression by the cell, inflammatory response suppression, and virus budding rate. In one embodiment, the agent is a small molecule. In one embodiment, the primate ICAM-1 is chimpanzee ICAM-1.

The present invention also includes a method to identify an agent which may modulate resistance to RNA virus, HCV virus, HIV-1-mediated disease, comprising contacting at least one agent to be tested with human ICAM-1, and detecting the increased or decreased dimerization of human ICAM-1, wherein an agent is identified by its ability to increase or decrease dimerization of the human ICAM-1 subunits whereby said increased or decreased dimerization of human ICAM-1 modulates resistance to RNA virus, HCV virus, HIV-1 modulated disease.

The present invention also includes a method to identify an agent which may modulate resistance to RNA virus, HCV virus, HIV-1-mediated disease, comprising contacting at least one agent to be tested with human ICAM-1, and detecting a change in ICAM-1 mediated cell to cell signaling, wherein an agent is identified by its ability to increase or decrease ICAM-1 mediated cell to cell signaling whereby said ICAM-1 mediated cell to cell signaling modulates resistance to RNA virus, HCV virus, HIV-1 modulated disease.

The term “transformation” or “transform” refers to any genetic modification of cells and includes both “transfection” and “transduction”. As used herein, “transfection of cells” refers to the acquisition by a cell of new genetic material by incorporation of added DNA. Thus, transfection refers to the insertion of nucleic acid (e.g., DNA) into a cell using physical or chemical methods. Several transfection techniques are known to those of ordinary skill in the art including: calcium phosphate DNA co-precipitation (Methods in Molecular Biology, Vol. 7, Gene Transfer and Expression Protocols, Ed. E. J. Murray, Humana Press (1991)); DEAE-dextran (supra); electroporation (supra); cationic liposome-mediated transfection (supra); and tungsten particle-facilitated microparticle bombardment (Johnston, S. A., Nature 346: 776-777 (1990)); and strontium phosphate DNA co-precipitation (Brash D. E. et al. Molec. Cell. Biol. 7: 2031-2034 (1987). Each of these methods is well represented in the art.

In contrast, “transduction of cells” refers to the process of transferring nucleic acid into a cell using a DNA or RNA virus. One or more isolated polynucleotide sequences encoding one or more proteins of the invention contained within the virus may be incorporated into the chromosome of the transduced cell. Alternatively, a cell is transduced with a virus but the cell will not have the isolated polynucleotide incorporated into its chromosomes but will be capable of expressing a protein of the invention extrachromosomally within the cell.

According to one embodiment, the cells are transformed (i.e., genetically modified) ex vivo. The cells are isolated from a mammal (preferably a human) and transformed (i.e., transduced or transfected in vitro) with a vector containing an isolated polynucleotide such as a recombinant gene operatively linked to one or more expression control sequences for expressing a recombinant protein of the invention. The cells are then administered to a mammalian recipient for delivery of the protein in situ. Preferably, the mammalian recipient is a human and the cells to be modified are autologous cells, i.e., the cells are isolated from the mammalian recipient. The isolation and culture of cells in vitro has been reported.

According to another embodiment, the cells are transformed or otherwise genetically modified in vivo. The cells from the mammalian recipient (preferably a human), are transformed (i.e., transduced or transfected) in vivo with a vector containing isolated polynucleotide such as a recombinant gene operatively linked to one or more expression control sequences for expressing a secreted protein (i.e., recombinant protein of the invention) and the protein is delivered in situ. The isolated polynucleotides encoding the protein (e.g., a cDNA encoding one or more therapeutic proteins of the invention) is introduced into the cell ex vivo or in vivo by genetic transfer methods, such as transfection or transduction, to provide a genetically modified cell. Various expression vectors (i.e., vehicles for facilitating delivery of the isolated polynucleotide into a target cell) are known to one of ordinary skill in the art. Typically, the introduced genetic material includes an isolated polynucleotide such as an gene of the invention(usually in the form of a cDNA comprising the exons coding for the protein of the invention) together with a promoter to control transcription of the new gene. The promoter characteristically has a specific nucleotide sequence necessary to initiate transcription. Optionally, the genetic material could include intronic sequences which will be removed from the mature transcript by RNA splicing. A polyadenylation signal should be present at the 3′ end of the gene to be expressed. The introduced genetic material also may include an appropriate secretion “signal” sequence for secreting the therapeutic gene product (i.e., a protein of the invention) from the cell to the extracellular milieu. Optionally, the isolated genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. Preferably, the isolated genetic material is introduced into the cell genome immediately downstream from the promoter so that the promoter and coding sequence are operatively linked so as to permit transcription of the coding sequence. Preferred viral expression vectors include an exogenous promoter element to control transcription of the inserted protein of the invention gene. Such exogenous promoters include both constitutive and inducible promoters. Naturally-occurring constitutive promoters control the expression of proteins that regulate essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, the .beta.-actin promoter (Lai et al., Proc. Natl. Acad. Sci. USA 86: 10006-10010 (1989)), and other constitutive promoters known to those of skill in the art.

In addition, many viral promoters function constitutively in eucaryotic cells. These include: the early and late promoters of SV40 (See Bernoist and Chambon, Nature, 290:304 (1981)); the long terminal repeats (LTRs) of Moloney Leukemia Virus and other retroviruses (See Weiss et al., RNA Tumor Viruses, Cold Spring Harbor Laboratory, Cold Spring Harbor, N.Y. (1985)); the thymidine-kinase promoter of Herpes Simplex Virus (HSV) (See Wagner et al., Proc. Nat. Acad. Sci. USA, 78: 1441 (1981)); the cytomegalovirus immediate-early (IE1) promoter (See Karasuyama et al., J. Exp. Med., 169: 13 (1989); the promoter of the Rous sarcoma virus (RSV) (Yamamoto et al., Cell, 22:787 (1980)); the adenovirus major late promoter (Yamada et al., Proc. Nat. Acad. Sci. USA, 82: 3567 (1985)), among many others. Accordingly, any of the above-referenced constitutive promoters can be used to control transcription of a gene insert. If delivery of the gene of the invention is to specific tissues, it may be desirable to target the expression of this gene. For instance, there are many promoters described in the literature which are only expressed in certain tissues. Examples include liver-specific promoters of hepatitis B virus (Sandig et al., Gene Therapy 3: 1002-1009 (1996) and the albumin gene (Pinkert et al., Genes and Development, 1: 268-276 (1987); see also Guo et al., Gene Therapy, 3: 802-810 (1996) for other liver-specific promoter. Moreover, there are many promoters described in the literature which are only expressed in specific tumors. Examples include the PSA promoter (prostate carcinoma), carcinoembryonic antigen promoter (colon and lung carcinoma), .beta.-casein promoter (mammary carcinoma), tyrosinase promoter (melanoma), calcineurin A. alpha. promoter (glioma, neuroblastoma), c-sis promoter (osteosarcoma) and the .alpha.-fetoprotein promoter (hepatoma). Genes that are under the control of inducible promoters are expressed only, or to a greater degree, in the presence of an inducing agent, (e.g., transcription under control of the metallothionein promoter is greatly increased in presence of certain metal ions). See also the glucocorticoid-inducible promoter present in the mouse mammary tumor virus long terminal repeat (MMTV LTR) (Klessig et al., Mol. Cell. Biol., 4: 1354 (1984)). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a gene of the invention in the genetically modified cell. If the gene encoding gene of the invention is under the control of an inducible promoter, delivery of the gene of the invention in situ is triggered by exposing the genetically modified cell in situ to conditions permitting transcription of the gene of the invention, e.g., by injection of specific inducers of the inducible promoters which control transcription of the agent. For example, in situ expression by genetically modified cells of protein encoded by an gene of the invention under the control of the metallothionein promoter is enhanced by contacting the genetically modified cells with a solution containing the appropriate (i.e., inducing) metal ions in situ.

Recently, very sophisticated systems have been developed which allow precise regulation of gene expression by exogenously administered small molecules. These include, the FK506/Rapamycin system (Rivera et al., Nature Medicine 2(9): 1028-1032, 1996); the tetracycline system (Gossen et al., Science 268: 1766-1768,1995), the ecdysone system (No et al., Proc. Nat. Acad. Sci., USA 93: 3346-3351,1996) and the progesterone system (Wang et al., Nature Biotechnology 15: 239-243,1997). Accordingly, the amount of a protein of the invention that is delivered in situ is regulated by controlling such factors as: (1) the nature of the promoter used to direct transcription of the inserted gene, (i.e., whether the promoter is constitutive or inducible, strong or weak or tissue specific); (2) the number of copies of the exogenous gene that are inserted into the cell; (3) the number of transduced/transfected cells that are administered (e.g., implanted) to the patient; (4) the size of an implant (e.g., graft or encapsulated expression system) in ex vivo methods; (5) the number of implants in ex vivo methods; (6) the number of cells transduced/transfected by in vivo administration; (7) the length of time the transduced/transfected cells or implants are left in place in both ex vivo and in vivo methods; and (8) the production rate of the protein of the invention by the genetically modified cell. Selection and optimization of these factors for delivery of a therapeutically effective dose of a particular protein of the invention is deemed to be within the scope of one of ordinary skill in the art without undue experimentation, taking into account the above-disclosed factors and the clinical profile of the patient. In addition to at least one promoter and at least one isolated polynucleotide encoding the protein of the invention, the expression vector may optionally include a selection gene, for example, a neomycin resistance gene, for facilitating selection of cells that have been transfected or transduced with the expression vector. Alternatively, the cells are transfected with two or more expression vectors, at least one vector containing the gene(s) encoding the gene of the invention, the other vector containing a selection gene. The selection of a suitable promoter, enhancer, selection gene and/or signal sequence (described below) is deemed to be within the scope of one of ordinary skill in the art without undue experimentation.

Any of the methods known in the art for the insertion of polynucleotide sequences into a vector may be used. See, for example, Sambrook et al., Molecular Cloning: A Laboratory Manual, Cold Spring Harbor Laboratory, Cold Spring Harbor, N.Y. (1989) and Ausubel et al., Current Protocols in Molecular Biology, J. Wiley & Sons, N.Y. (1992). Conventional vectors consist of appropriate transcriptional/translational control signals operatively linked to the polynucleotide sequence for a particular protein of the invention. Promoters/enhancers may also be used to control expression of proteins of the invention.

Expression vectors compatible with mammalian host cells for use in gene therapy of tumor cells include, for example, plasmids; avian, murine and human retroviral vectors; adenovirus vectors; herpes viral vectors; parvoviruses; and non-replicative pox viruses. In particular, replication-defective recombinant viruses can be generated in packaging cell lines that produce only replication-defective viruses. See Current Protocols in Molecular Biology: Sections 9.10-9.14 (Ausubel et al., eds.), Greene Publishing Associates, 1989. Specific viral vectors for use in gene transfer systems are now well established. See for example: Madzak et al., J. Gen. Virol., 73: 1533-36 (1992) (papovavirus SV40); Berkner et al., Curr. Top. Microbiol. Immunol., 158: 39-61 (1992) (adenovirus); Moss et al., Curr. Top. Microbiol. Immunol., 158: 25-38 (1992) (vaccinia virus); Muzyczka, Curr. Top. Microbiol. Immunol., 158: 97-123 (1992) (adeno-associated virus); Margulskee, Curt. Top. Microbiol. Immunol., 158: 67-93 (1992) (herpes simplex virus (HSV) and Epstein-Barr virus (HBV)); Miller, Curr. Top. Microbiol. Immunol., 158:1-24 (1992) (retrovirus); Brandyopadhyay et at., Mol. Cell. Biol., 4: 749-754 (1984) (retrovirus); Miller et al., Nature, 357: 455-460 (1992) (retrovirus); Anderson, Science, 256: 808-813 (1992) (retrovirus). In one embodiment, vectors are DNA viruses that include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al., Gene Therapy 1: 367-384,1994; U.S. Pat. Nos. 4,797,368 and 5,399,346 and discussion below. The choice of a particular vector system for transferring, for instance, a protein of the invention sequence will depend on a variety of factors. One important factor is the nature of the target cell population. Although retroviral vectors have been extensively studied and used in a number of gene therapy applications, they are generally unsuited for infecting cells that are not dividing but may be useful in cancer therapy since they only integrate and express their genes in replicating cells. They are useful for ex vivo approaches and are attractive in this regard due to their stable integration into the target cell genome.

Adenoviruses are eukaryotic DNA viruses that can be modified to efficiently deliver a therapeutic or reporter transgene to a variety of cell types. The general adenoviruses types 2 and 5 (Ad2 and Ad5, respectively), which cause respiratory disease in humans, are currently being developed for gene therapy of Duchenne Muscular Dystrophy (DMD) and Cystic Fibrosis (CF). Both Ad2 and Ad5 belong to a subclass of adenovirus that are not associated with human malignancies. Adenovirus vectors are capable of providing extremely high levels of transgene delivery to virtually all cell types, regardless of the mitotic state. High titers (10¹¹ plaque forming units/ml) of recombinant virus can be easily generated in 293 cells (an adenovirus-transformed, complementation human embryonic kidney cell line: ATCC CRL1573) and cryo-stored for extended periods without appreciable losses. The efficiency of this system in delivering a therapeutic transgene in vivo that complements a genetic imbalance has been demonstrated in animal models of various disorders. See Y. Watanabe, Atherosclerosis, 36: 261-268 (1986); K Tanzawa et al, FEBS letters, 118(1):81-84 (1980); J. L. Golasten et al, New Engl. J. Med., 309 (11983): 288-296 (1983); S. Ishibashi et al, J. Clin. Invest., 92: 883-893 (1993); and S. Ishibashi et al, J. Clin. Invest., 93: 1889-1893 (1994). Indeed, recombinant replication defective adenovirus encoding a cDNA for the cystic fibrosis transmembrane regulator (CFTR) has been approved for use in several human CF clinical trials. See, e.g., J. Wilson, Nature, 365: 691-692 (Oct., 21, 1993). Further support of the safety of recombinant adenoviruses for gene therapy is the extensive experience of live adenovirus vaccines in human populations. Human adenoviruses are comprised of a linear, approximately 36 kb double-stranded DNA genome, which is divided into 100 map units (m.u.), each of which is 360 bp in length. The DNA contains short inverted terminal repeats (ITR) at each end of the genome that are required for viral DNA replication. The gene products are organized into early (E1 through E4) and late (L1 through L5) regions, based on expression before or after the initiation of viral DNA synthesis. See, e.g., Horwitz, Virology, 2d edit., ed. B. N. Fields, Raven Press Ltd., New York (1990). The adenovirus genome undergoes a highly regulated program during its normal viral life cycle. See Y. Yang et, al Proc. Natl. Acad. Sci. U.S.A, 91: 4407-4411 (1994). Virions are internalized by cells, enter the endosome, and from there the virus enters the cytoplasm and begins to lose its protein coat. The virion DNA migrates to the nucleus, where it retains its extrachromosomal linear structure rather than integrating into the chromosome. The immediate early genes, E1a and E1b, are expressed in the nucleus. These early gene products regulate adenoviral transcription and are required for viral replication and expression of a variety of host genes (which prime the cell for virus production), and are central to the cascade activation of delayed early genes (e.g. E2, E3, and E4) followed by late genes (e.g. L1-L5). The first-generation recombinant, replication-deficient adenoviruses which have been developed for gene therapy contain deletions of the entire E1a and part of the E 11b regions. This replication-defective virus is grown in 293 cells which contain a functional adenovirus E1 region which provides in trans E1 proteins, thereby allowing replication of E1-deleted adenovirus. The resulting virus is capable of infecting many cell types and can express the introduced gene (providing it carries a promoter), but cannot replicate in a cell that does not carry the E1 region DNA. Recombinant adenoviruses have the advantage that they have a broad host range, can infect quiescent or terminally differentiated cells such as neurons, and appear essentially non-oncogenic. Adenoviruses do not appear to integrate into the host genome. Because they exist extrachromasomally, the risk of insertional mutagenesis is greatly reduced. Recombinant adenoviruses produce very high titers, the viral particles are moderately stable, expression levels are high, and a wide range of cells can be infected.

Adeno-associated viruses (AAV) have also been employed as vectors for somatic gene therapy. AAV is a small, single-stranded (ss) DNA virus with a simple genomic organization (4.7 kb) that makes it an ideal substrate for genetic engineering. Two open reading frames encode a series of rep and cap polypeptides. Rep polypeptides (rep78, rep68, rep 62 and rep 40) are involved in replication, rescue and integration of the AAV genome. The cap proteins (VP 1, VP2 and VP3) form the virion capsid. Flanking the rep and cap open reading frames at the 5′ and 3′ ends are 145 bp inverted terminal repeats (ITRs), the first 125 bp of which are capable of forming Y- or T-shaped duplex structures. Of importance for the development of AAV vectors, the entire rep and cap domains can be excised and replaced with a therapeutic or reporter transgene. See B. J. Carter, in Handbook of Parvoviruses, ed., P. Tijsser, CRC Press, pp. 155-168 (1990). It has been shown that the ITRs represent the minimal sequence required for replication, rescue, packaging, and integration of the AAV genome. The AAV life cycle is biphasic, composed of both latent and lytic episodes. During a latent infection, AAV virions enter a cell as an encapsidated ssDNA, and shortly thereafter are delivered to the nucleus where the AAV DNA stably integrates into a host chromosome without the apparent need for host cell division. In the absence of a helper virus, the integrated AAV genome remains latent but capable of being activated and rescued. The lytic phase of the life cycle begins when a cell harboring an AAV provirus is challenged with a secondary infection by a herpesvirus or adenovirus which encodes helper functions that are required by AAV to aid in its excision from host chromatin (B. J. Carter, supra). The infecting parental single-stranded (ss) DNA is expanded to duplex replicating form (RF) DNAs in a rep dependent manner. The rescued AAV genomes are packaged into preformed protein capsids (icosahedral symmetry approximately 20 nm in diameter) and released as infectious virions that have packaged either + or − ssDNA genomes following cell lysis. The viral particles are very stable and recombinant AAVs (rAAV) have “drug-like” characteristics in that rAAV can be purified by pelleting or by CsCl gradient banding. They are heat stable and can be lyophilized to a powder and rehydrated to full activity. Their DNA stably integrates into host chromosomes so expression is long-term. Their host range is broad and AAV causes no known disease so that the recombinant vectors are non-toxic. High level gene expression from AAV in mice was shown to persist for at least 1.5 years. See Xiao, Li and Samuiski (1996) Journal of Virology 70, 8089-8108. Since there was no evidence of viral toxicity or a cellular host immune response, these limitations of viral gene therapy have been overcome. Kaplitt, Leone, Samulski, Xiao, Pfaff, O'Malley and During (1994) Nature Genetics 8, 148-153 described long-term (up to 4 months) expression of tyrosine hydroxylase in the rat brain following direct intracranial injection using an AAV vector. This is a potential therapy for Parkinson's Disease in humans. Expression was highly efficient and the virus was safe and stable. Fisher et al. (Nature Medicine (1997) 3, 306-312) reported stable gene expression in mice following injection into muscle of AAV. Again, the virus was safe. No cellular or humoral immune response was detected against the virus or the foreign gene product. Kessler et al. (Proc. Natl. Acad. Sci. USA (1996) 93, 14082-14087) showed high-level expression of the erythropoietin (Epo) gene following intramuscular injection of AAV in mice. Epo protein was demonstrated to be present in circulation and an increase in the red blood cell count was reported, indicative of therapeutic potential. Other work by this group has used AAV expressing the HSV tk gene as a treatment for cancer. High level gene expression in solid tumors has been described.

Recently, recombinant baculovirus, primarily derived from the baculovirus Autographa californica multiple nuclear polyhedrosis virus (AcMNPV), has been shown to be capable of transducing mammalian cells in vitro. (See Hofmann, C., Sandig, V., Jennings, G., Rudolph, M., Schlag, P., and Strauss, M. (1995), “Efficient gene transfer into human hepatocytes by baculovirus vectors”, Proc. Natl. Acad. Sci. USA 92, 10099-10103; Boyce, F. M. and Bucher, N. L. R. (1996) “Baculovirus-mediated gene transfer into mammalian cells”, Proc. Natl. Acad. Sci. USA 93, 2348-2352). Recombinant baculovirus has several potential advantages for gene therapy. These include a very large DNA insert capacity, a lack of a preexisting immune response in humans, lack of replication in mammals, lack of toxicity in mammals, lack of expression of viral genes in mammalian cells due to the insect-specificity of the baculovirus transcriptional promoters, and, potentially, a lack of a cytotoxic T lymphocyte response directed against these viral proteins.

### Description of the HCV Embodiment (an Example of a Positively Selected Non-Human Trait)

Some four million Americans are infected with the hepatitis C virus (HCV), and worldwide, the number approaches 40 million (Associated Press, Mar. 11, 1999). Many of these victims are unaware of the infection, which can lead to hepatocellular carcinoma. This disease is nearly always fatal. Roughly 14,500 Americans die each year as a result of the effects of hepatocellular carcinoma (Associated Press, Mar. 11, 1999). Thus identification of therapeutic agents that can ameliorate the effects of chronic infection are valuable both from an ethical and commercial viewpoint.

The chimpanzee is the only organism, other than humans, known to be susceptible to HCV infection (Lanford, R. E. et al. (1991) J. Med. Virol. 34:148-153). While the original host population for HCV has not yet been documented, it is likely that the virus must have originated in either humans or chimpanzees, the only two known susceptible species. It is known that the continent-of-origin for HCV is Africa (personal communication, A. Siddiqui, University of Colorado Health Science Center, Denver). If the chimpanzee population were the original host for HCV, as many HCV researchers believe (personal communication, A. Siddiqui, University of Colorado Health Science Center), then, as is known to be true for the HIV virus, chimpanzees would likely have evolved resistance to the virus. This hypothesis is supported by the well-documented observation that HCV-infected chimpanzees are refractory to the hepatic damage that often occurs in hepatitis C-infected humans (Walker, C. M (1997) Springer Semin. Immunopathol. 19:85-98; McClure, H. M., pp. 121-133 in The Role of the Chimpanzee in Research, ed. by Eder, G. et al., 1994, Basel: Karger; Agnello, V. et al. (1998) Hepatology 28:573-584). In fact, although in 2% of HCV-infected humans, the disease course leads to hepatocellular carcinoma, HCV-infected chimpanzees do not develop these tumors (Walker, C. M (1997) Springer Semin. Immunopathol. 19:85-98). Further support for the hypothesis that chimpanzees were the original host population, and that they have, as a result of prolonged experience with the virus, evolved resistance to the ravages of HCV-induced disease, is added by the observation that HCV-infected chimpanzees in general have a milder disease course (i.e., not simply restricted to hepatic effects) than do humans (Lanford, R. E. et al. (1991) J. Med. Virol. 34:148-153; and Walker, C. M (1997) Springer Semin. Immunopathol. 19:85-98).

As is exemplified herein, the p44 gene in chimpanzees has been positively selected relative to its human homolog. The p44 protein was first identified in liver tissues of chimpanzees experimentally infected with HCV (Shimizu, Y. et al. (1985) PNAS USA 82:2138).

The p44 gene, and the protein it codes for, represents a potential therapeutic target, or alternatively a route to a therapeutic, for humans who are chronically infected with hepatitis C. The protein coded for by this gene in chimpanzees is known to be up-regulated in chimpanzee livers after experimental infection of captive chimpanzees (Takahashi, K. et al. (1990) J. Gen. Virol. 71:2005-2011). The p44 gene has been shown to be a member of the family of A/l interferon inducible genes (Kitamura, A. et al. (1994) Eur. J. Biochem. 224:877-883). It is suspected that the p44 protein is a mediator in the antiviral activities of interferon.

This is most suggestive, since as noted above, HCV-infected chimpanzees have been documented to be refractory to the hepatic damage that often occurs in HCV-infected humans. The combination of the observations that this protein is only expressed in chimpanzee livers after hepatitis C infection, the fact that chimpanzees are refractory to the hepatic damage that can occur in humans (Agnello, V. et al. (1998) Hepatology 28:573-584), the observation that HCV-infected chimpanzees in general have a milder disease course than do humans, and that the p44 gene has been positively selected in chimpanzees, strongly suggest that the chimpanzee p44 protein confers resistance to hepatic damage in chimpanzees. Whether the protein is responsible for initiating some type of cascade in chimpanzees that fails to occur in infected humans, or whether the selected chimpanzee homolog differs in some critical biochemical functions from its human homolog, is not yet clear. It has been speculated that the milder disease course observed in chimpanzees may be due in part to lower levels of viral replication (Lanford, R. E. et al. (1991) J. Med. Virol. 34:148-153).

This invention includes the medical use of the specific amino acid residues by which chimpanzee p44 differs from human p44. These residues that were positively selected during the period in which chimpanzees evolved an accommodation to the virus, allow the intelligent design of an effective therapeutic approach for chronically HCV-infected humans. Several methods to induce a chimpanzee-like response in infected humans will be apparent to one skilled in the art. Possibilities include the intelligent design of a small molecule therapeutic targeted to the human homolog of the specific amino acid residues selected in chimpanzee evolution. Use of molecular modeling techniques might be valuable here, as one could design a small molecule that causes the human protein to mimic the three-dimensional structure of the chimpanzee protein. Another approach would be the design of a small molecule therapeutic that induces a chimpanzee-like functional response in human p44. Again, this could only be achieved by use of the knowledge obtained by this invention, i.e., which amino acid residues were positively selected to confer resistance to HCV in chimpanzees. Other possibilities will be readily apparent to one skilled in the art.

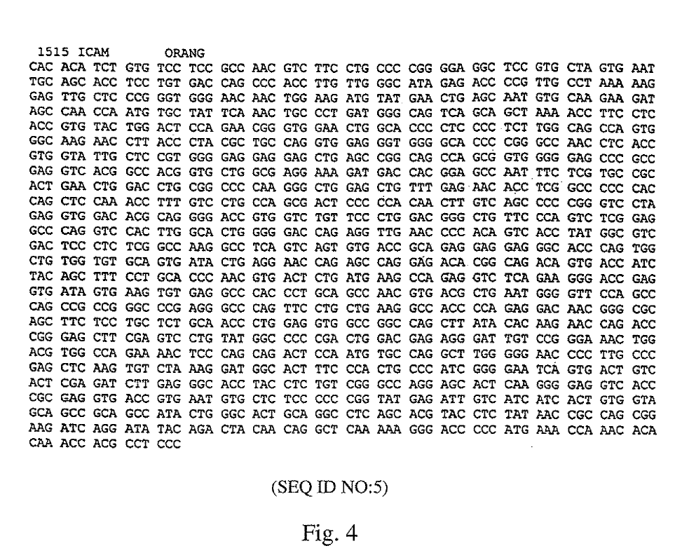

In addition to screening candidate agents for those that may favorably interact with the human p44 (exon 2) polypeptide so that it may mimic the structure and/or function of chimpanzee p44, the subject invention also concerns the screening of candidate agents that interact with the human p44 polynucleotide promoter, whereby the expression of human p44 may be increased so as to improve the human patient's resistance to HCV infection. Thus, the subject invention includes a method for identifying an agent that modulates expression of a human's p44 polynucleotide, by contacting at least one candidate agent with the human's p44 polynucleotide promoter, and observing whether expression of the human p44 polynucleotide is enhanced. The human p44 promoter has been published in Kitamura et al. (1994) Eur. J. Biochem. 224:877 (FIG. 4).

### Description of the Breast Enhancement Embodiment (an Example of a Positively Selected Human Trait)

Relative to non-human primates, female humans exhibit pre-pregnancy, pre-lactation expanded breast tissue. As is discussed in the Examples, this secondary sex characteristic is believed to facilitate evolved behaviors in humans associated with long term pair bonds and long-term rearing of infants. One aspect of this invention concerns identifying those human genes that have been positively selected in the development of enlarged breasts. Specifically, this invention includes a method of determining whether a human polynucleotide sequence which has been associated with enlarged breasts in humans has undergone evolutionarily significant change relative to a non-human primate that does not manifest enlarged breasts, comprising: a) comparing the human polynucleotide sequence with the corresponding non-human primate polynucleotide sequence to identify any nucleotide changes; and b) determining whether the human nucleotide changes are evolutionarily significant.

It has been found that the human BRCA1 gene, which has been associated with normal breast development in humans, has been positively selected relative to the BRCA1 gene of chimpanzees and other non-human primates. The identified evolutionarily significant nucleotide changes could be useful in developing agents that can modulate the function of the BRCA1 gene or protein.

Therapeutic Compositions that Comprise Agents

As described herein, agents can be screened for their capacity to increase or decrease the effectiveness of the positively selected polynucleotide or polypeptide identified according to the subject methods. For example, agents that may be suitable for enhancing breast development may include those which interact directly with the BRCA1 protein or its ligand, or which block inhibitors of BRCA1 protein. Alternatively, an agent may enhance breast development by increasing BRCA1 expression. As the mechanism of BRCA1 is further elucidated, strategies for enhancing its efficacy can be devised.

In another example, agents that may be suitable for reducing the progression of AIDS could include those which directly interact with the human CD59 protein in a manner to make the protein unusable to the HIV virion, possibly by either rendering the human CD59 unsuitable for packing in the virion particle or by changing the orientation of the protein with respect to the cell membrane (or via some other mechanism). The candidate agents can be screened for their capacity to modulate CD59 function using an assay in which the agents are contacted with HIV infected cells which express human CD59, to determine whether syncytia formation or other indicia of the progression of AIDS are reduced. The assay may permit the detection of whether the HIV virion can effectively pack the CD59 and/or utilize the CD59 to inhibit attack by MAC complexes.

One agent that may slow AIDS progression is a human CD59 that has been modified to have multiple GPI links. As described herein, chimp CD59, which contains three GPI links as compared to the single GPI link found in human CD59, slows progression of HIV infections in chimps. Preferably, the modified human CD59 contains three GPI links in tandem.

Another example of an agent that may be suitable for reducing AIDS progression is a compound that directly interacts with human DC-SIGN to reduce its capacity to bind to HIV-1 and transport it to the lymph nodes. Such an agent could bind directly to the HIV-1 binding site on DC-SIGN. The candidate agents can be contacted with dendritic cells expressing DC-SIGN or with a purified extracellular fragment of DC-SIGN and tested for their capacity to inhibit HIV-1 binding.

Various delivery systems are known in the art that can be used to administer agents identified according to the subject methods. Such delivery systems include aqueous solutions, encapsulation in liposomes, microparticles or microcapsules or conjugation to a moiety that facilitates intracellular admission.

Therapeutic compositions comprising agents may be administered parenterally by injection, although other effective administration forms, such as intra-articular injection, inhalant mists, orally-active formulations, transdermal iontophoresis or suppositories are also envisioned. The carrier may contain other pharmacologically-acceptable excipients for modifying or maintaining the pH, osmolarity, viscosity, clarify, color, sterility, stability, rate of dissolution, or odor of the formulation. The carrier may also contain other pharmacologically-acceptable excipients for modifying or maintaining the stability, rate of dissolution, release or absorption of the agent. Such excipients are those substances usually and customarily employed to formulate dosages for parenteral administration in either unit dose or multi-dose form.

Once the therapeutic composition has been formulated, it may be stored in sterile vials as a solution, suspension, gel, emulsion, solid, or dehydrated or lyophilized powder. Such formulations may be stored either in a ready to use form or requiring reconstitution immediately prior to administration. The manner of administering formulations containing agents for systemic delivery may be via subcutaneous, intramuscular, intravenous, intranasal or vaginal or rectal suppository. Alternatively, the formulations may be administered directly to the target organ (e.g., breast).

The amount of agent which will be effective in the treatment of a particular disorder or condition will depend on the nature of the disorder or condition, which can be determined by standard clinical techniques. In addition, in vitro or in vivo assays may optionally be employed to help identify optimal dosage ranges. The precise dose to be employed in the formulation will also depend on the route of administration, and the seriousness or advancement of the disease or condition, and should be decided according to the practitioner and each patient's circumstances. Effective doses may be extrapolated from dose-response curves derived from in vitro or animal model test systems. For example, an effective amount of an agent identified according to the subject methods is readily determined by administering graded doses of a bivalent compound of the invention and observing the desired effect.

Description of a Method for Obtaining Candidate Polynucleotides that May Be Associated with Human Diseases, and Diagnostic Methods Derived Therefrom

According to the subject invention, BRCA1 exon 11 is an evolutionarily significant polynucleotide that has undergone positive selection in humans relative to chimpanzees, and is associated with the enhanced breast development observed in humans relative to chimpanzees (see Example 14). Exon 11 has also been found to have mutations that are associated with the development of breast cancer. BRCA1 exon 11 mutations are known to be associated with both familial and spontaneous breast cancers (Kachhap, S. K. et al. (2001) Indian J. Exp. Biol. 39(5):391-400; Hadjisavvas, A. et al. (2002) Oncol. Rep. 9(2):383-6; Khoo, U. S. et al. (1999) Oncogene 18(32):4643-6).

Encompassed within the subject invention are methods that are based on the principle that human polynucleotides that are evolutionarily significant relative to a non-human primate, and which are associated with a improved physiological condition in the human, may also be associated with decreased resistance or increased susceptibility to one or more diseases. In one embodiment, mutations in positively selected human BRCA1 polynucleotide exon 11 may be linked to elevated risk of breast, ovarian and/or prostate cancer. This phenomenon may represent a trade-off between enhanced development of one trait and loss or reduction in another trait in polynucleotides encoding polypeptides of multiple functions. In this way, identification of positively selected human polynucleotides can serve to identify a pool of genes that are candidates for susceptibility to human diseases.

Thus, in one embodiment, the subject invention provides a method for obtaining a pool of candidate polynucleotides that are useful in screening for identification of polynucleotides associated with increased susceptibility or decreased resistance to one or more human diseases. The method of identifying the candidate polynucleotides comprises comparing the human polynucleotide sequences with non-human primate polynucleotide sequences to identify any nucleotide changes, and determining whether those nucleotide changes are evolutionarily significant. Evolutionary significance can be determined by any of the methods described herein including the K_{A}/K_{S} method. Because evolutionary significance involves the number of non-silent nucleotide changes over a defined length of polynucleotide, it is the polynucleotide containing the group of nucleotide changes that is referred to herein as “evolutionarily significant.” That is, a single nucleotide change in a human polynucleotide relative to a non-human primate cannot be analyzed for evolutionary significance without considering the length of the polynucleotide and the existence or (non-existence) of other non-silent nucleotide changes in the defined polynucleotide. Thus, in referring to an “evolutionarily significant polynucleotide” and the nucleotide changes therein, the size of the polynucleotide is generally considered to be between about 30 and the total number of nucleotides encompassed in the polynucleotide or gene sequence (e.g., up to 3,000-5,000 nucleotides or longer). Further, while individual nucleotide changes cannot be analyzed in isolation as to their evolutionary significance, nucleotide changes that contribute to the evolutionary significance of a polynucleotide are referred to herein as “evolutionarily significant nucleotide changes.”

The subject method further comprises a method of correlating an evolutionarily significant nucleotide change in a candidate polynucleotide to decreased resistance to development of a disease in humans, comprising identifying evolutionarily significant candidate polynucleotides as described herein, and further analyzing the functional effect of the evolutionarily significant nucleotide change(s) in one or more of the candidate polynucleotides in a suitable model system, wherein the presence of a functional effect indicates a correlation between the evolutionarily significant nucleotide change in the candidate polynucleotide and the decreased resistance to development of the disease in humans. As discussed herein, model systems may be cell-based or in vivo. For example, the evolutionarily significant human BRCA1 exon 11 (or variations thereof having fewer evolutionarily significant nucleotide changes) could be transfected or knock-out genomically inserted into mice or non-human primates (e.g., chimpanzees) to determine if it induces the functional effect of breast, ovarian or prostate cancer in the test animals. Such test results would indicate whether specific evolutionarily significant changes in exon 11 are associated with increased incidence of breast, ovarian or prostate cancer.

In addition to evaluating the evolutionarily significant nucleotide changes in candidate polynucleotides for their relevance to development of disease, the subject invention also includes the evaluation of other nucleotide changes of candidate human polynucleotides, such as alleles or mutant polynucleotides, that may be responsible for the development of the disease. For example, the evolutionarily significant BRCA1 exon 11 has a number of allelic or mutant exon 11s in human populations that have been found to be associated with breast, ovarian or prostate cancer (Rosen, E. M. et al. (2001) Cancer Invest. 19(4):396-412; Elit, L. et al. (2001) Int. J. Gynecol. Cancer 11(3):241-3; Shen, D. et al. (2000) J. Natl. Med. Assoc. 92(1):29-35; Khoo, U. S. et al. (1999) Oncogene 18(32):4643-6; Presneau, N. et al. (1998) Hum. Genet. 103(3):334-9; Dong, J. et al. (1998) Hum. Genet. 103(2):154-61; and Xu, C. F. et al. (1997) Genes Chromosomes 18(2):102-10). For example, Grade, K. et al. (1996) J. Cancer Res. Clin. Oncol. 122(11):702-6, report that of 127 human BRCA1 mutations published by 1996, 55% of them are localized in exon 11. Many of the cancer-causing mutations in BRCA1 exon 11 are not considered to be predominantly present in humans, and are therefore not considered to contribute to the evolutionarily significance of BRCA1 exon 11. Polynucleotides that are strongly positively selected for the development of one trait in humans may be hotspots for nucleotide changes (evolutionarily significant or otherwise) that are associated with the development of a disease. Thus, according to the subject invention, identification of candidate polynucleotides that have been positively selected, is a very efficient start to identifying corresponding mutant or allelic polynucleotides associated with a disease.

To identify whether mutants or alleles of evolutionarily significant polynucleotides in humans can be correlated to decreased resistance or increased susceptibility to the disease, the variant polynucleotide can be tested in a suitable model, such as the MCF10a normal human epithelial cell line (Favy, D A et al. (2001) Biochem. Biophys. Res. Commun. 274(1):73-8). This model system for breast cancer can involve transfection of or knock-out genomic insertion into the MCF10a normal human breast epithelial cell line with mutant or allelic BRCA1 exon 11 polynucleotides to determine whether the nucleotide changes in the mutant or allelic polynucleotides result in conversion of the cell line to a neoplastic phenotype, i.e., a phenotype similar to cancer cell lines MCF-7, MDA-MB231 or HBL100 (Favy et al., supra). Additionally, mutants of candidate polynucleotides can be compared to patient genetic data to determine whether, for example, BRCA1 exon 11 mutant nucleotide changes are present in familial and/or sporadic breast, ovarian and/or prostate tumors. In this way, mutations in candidate evolutionarily significant human polynucleotides can be evaluated for their functional effect and their correlation to development of breast, ovarian and/or prostate cancer in humans.

The following examples are provided to further assist those of ordinary skill in the art. Such examples are intended to be illustrative and therefore should not be regarded as limiting the invention. A number of exemplary modifications and variations are described in this application and others will become apparent to those of skill in this art. Such variations are considered to fall within the scope of the invention as described and claimed herein.

### EXAMPLES

### Example 1

### cDNA Library Construction

A chimpanzee cDNA library is constructed using chimpanzee tissue. Total RNA is extracted from the tissue (RNeasy kit, Quiagen; RNAse-free Rapid Total RNA kit, 5 Prime-3 Prime, Inc.) and the integrity and purity of the RNA are determined according to conventional molecular cloning methods. Poly A+ RNA is isolated (Mini-Oligo(dT) Cellulose Spin Columns, 5 Prime-3 Prime, Inc.) and used as template for the reverse-transcription of cDNA with oligo (dT) as a primer. The synthesized cDNA is treated and modified for cloning using commercially available kits. Recombinants are then packaged and propagated in a host cell line. Portions of the packaging mixes are amplified and the remainder retained prior to amplification. The library can be normalized and the numbers of independent recombinants in the library is determined.

### Example 2

### Sequence Comparison

Suitable primers based on a candidate human gene are prepared and used for PCR amplification of chimpanzee cDNA either from a cDNA library or from cDNA prepared from mRNA. Selected chimpanzee cDNA clones from the cDNA library are sequenced using an automated sequencer, such as an ABI 377. Commonly used primers on the cloning vector such as the M13 Universal and Reverse primers are used to carry out the sequencing. For inserts that are not completely sequenced by end sequencing, dye-labeled terminators are used to fill in remaining gaps.

The detected sequence differences are initially checked for accuracy, for example by finding the points where there are differences between the chimpanzee and human sequences; checking the sequence fluorogram (chromatogram) to determine if the bases that appear unique to human correspond to strong, clear signals specific for the called base; checking the human hits to see if there is more than one human sequence that corresponds to a sequence change; and other methods known in the art, as needed. Multiple human sequence entries for the same gene that have the same nucleotide at a position where there is a different chimpanzee nucleotide provides independent support that the human sequence is accurate, and that the chimpanzee/human difference is real. Such changes are examined using public database information and the genetic code to determine whether these DNA sequence changes result in a change in the amino acid sequence of the encoded protein. The sequences can also be examined by direct sequencing of the encoded protein.

### Example 3

### Molecular Evolution Analysis

The chimpanzee and human sequences under comparison are subjected to K_{A}/K_{S} analysis. In this analysis, publicly available computer programs, such as Li 93 and INA, are used to determine the number of non-synonymous changes per site (K_{A}) divided by the number of synonymous changes per site (K_{S}) for each sequence under study as described above. Full-length coding regions or partial segments of a coding region can be used. The higher the K_{A}/K_{S} ratio, the more likely that a sequence has undergone adaptive evolution. Statistical significance of K_{A}/K_{S} values is determined using established statistic methods and available programs such as the t-test.

To further lend support to the significance of a high K_{A}/K_{S} ratio, the sequence under study can be compared in multiple chimpanzee individuals and in other non-human primates, e.g., gorilla, orangutan, bonobo. These comparisons allow further discrimination as to whether the adaptive evolutionary changes are unique to the human lineage compared to other non-human primates. The sequences can also be examined by direct sequencing of the gene of interest from representatives of several diverse human populations to assess to what degree the sequence is conserved in the human species.

### Example 4

### Identification of Positively Selected ICAM-1, ICAM-2 and ICAM-3

Using the methods of the invention described herein, the intercellular adhesion molecules ICAM-1, ICAM-2 and ICAM-3 have been shown to have been strongly positively selected. The ICAM molecules are involved in several immune response interactions and are known to play a role in progression to AIDS in HIV infected humans. The ICAM proteins, members of the Ig superfamily, are ligands for the integrin leukocyte associated function 1 molecule (LFA-1). Makgoba et al (1988) Nature 331:86-88. LFA-1 is expressed on the surface of most leukocytes, while ICAMs are expressed on the surface of both leukocytes and other cell types. Larson et al. (1989) J. Cell Biol. 108:703-712. ICAM and LFA-1 proteins are involved in several immune response interactions, including T-cell function, and targeting of leukocytes to areas of inflammation. Larson et al. (1989).

Total RNA was prepared using either the RNeasy® kit (Qiagen), or the RNAse-free Rapid Total RNA kit (5 Prime -3 Prime, Inc.) from primate tissues (chimpanzee brain and blood, gorilla blood and spleen, orangutan blood) or from cells harvested from the following B lymphocyte cell lines: CARL (chimpanzee), ROK (gorilla), and PUTI (orangutan). mRNA was isolated from total RNA using the Mini-Oligo(dT) Cellulose Spin Columns (5 Prime -3 Prime, Inc.). cDNA was synthesized from mRNA with oligo dT and/or random priming using the cDNA Synthesis Kit (Stratagene®). The protein-coding region of the primate ICAM-1 gene was amplified from cDNA using primers (concentration=100 nmole/μl) designed by hand from the published human sequence. PCR conditions for ICAM-1 amplification were 94° C. initial pre-melt (4 min), followed by 35 cycles of 94° C. (15 sec), 58° C. (1 min 15 sec), 72° C. (1 min 15 sec), and a final 72° C. extension for 10 minutes. PCR was accomplished using Ready-to-Go™ PCR beads (Amersham Pharmacia Biotech) in a 50 microliter total reaction volume. Appropriately-sized products were purified from agarose gels using the QiaQuick® Gel Extraction kit (Qiagen). Both strands of the amplification products were sequenced directly using the Big Dye Cycle Sequencing Kit and analyzed on a 373A DNA sequencer (ABI BioSystems).

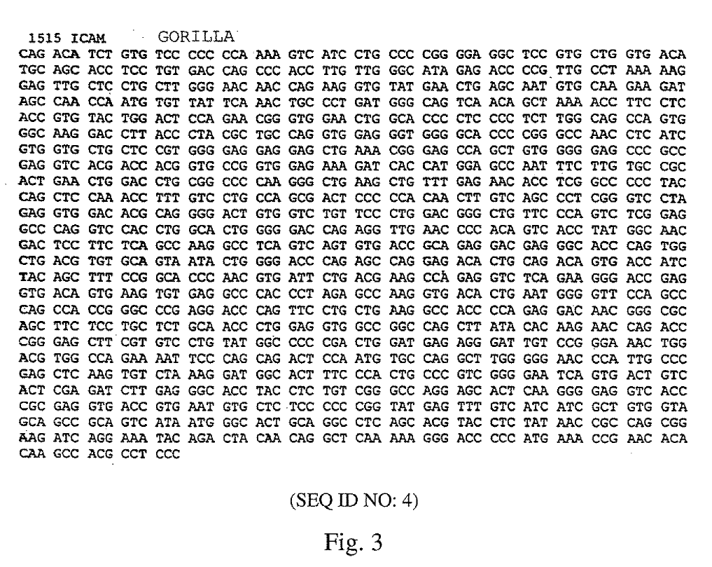

Comparison of the protein-coding portions of the human, gorilla (Gorilla gorilla), and orangutan (Pongo pygmaeus) ICAM-1 genes to that of the chimpanzee yielded statistically significant K_{A}/K_{S} ratios (Table 2). The protein-coding portions of the human and chimpanzee ICAM-1 genes were previously published and the protein-coding portions of gorilla (Gorilla gorilla), and orangutan (Pongo pygmaeus) ICAM-1 genes are shown in FIGS. 3 and 4, respectively.

For this experiment, pairwise K_{A}/K_{S} ratios were calculated for the mature protein using the algorithm of Li (1985; 1993). Statistically significant comparisons (determined by t-tests) are shown in bold. Although the comparison to gorilla and human was sufficient to demonstrate that chimpanzee ICAM-1 has been positively-selected, the orangutan ICAM-1 was compared as well, since the postulated historical range of gorillas in Africa suggests that gorillas could have been exposed to the HIV-1 virus. Nowak and Paradiso (1983) Walker=s Mammals of the World (Baltimore, Md., The Johns Hopkins University Press). The orangutan, however, has always been confined to Southeast Asia and is thus unlikely to have been exposed to HIV over an evolutionary time frame. (Nowak and Paradiso, 1983) (Gorillas are most closely-related to humans and chimpanzees, while orangutans are more distantly-related.)

TABLE 2
K_{A}/K_{S} Ratios: ICAM-1 Whole Protein Comparisons

| Species Compared | K_{A}/K_{S} Ratio |
|---|---|
| Chimpanzee to Human | 2.1 (P < 0.01) |
| Chimpanzee to Gorilla | 1.9 (P < 0.05) |
| Chimpanzee to Orangutan | 1.4 (P < 0.05) |
| Human to Gorilla | 1.0 |
| Human to Orangutan | 0.87 |
| Gorilla to Orangutan | 0.95 |

Even among those proteins for which positive selection has been demonstrated, few show K_{A}/K_{S} ratios as high as these ICAM-1 comparisons. Lee and Vacquier (1992) Biol. Bull. 182:97-104; Swanson and Vacquier (1995) Proc. Natl. Acad. Sci. USA 92:4957-4961; Messier and Stewart (1997); Sharp (1997) Nature 385:111-112. The results are consistent with strong selective pressure resulting in adaptive changes in the chimpanzee ICAM-1 molecule.

The domains (D1 and D2) of the ICAM-1 molecule which bind to LFA-1 have been documented. Staunton et al. (1990). Cell 61:243-254. Pairwise K_{A}/K_{S} comparisons between primate ICAM-1 genes. K_{A}/K_{S} ratios were calculated for domains D1 and D2 only, using the algorithm of Li (1985; 1993) (Table 3). Statistically significant comparisons (determined by t-tests) are shown in bold. The very high, statistically significant K_{A}/K_{S} ratios for domains D1 and D2 suggest that these regions of the protein were very strongly positively-selected. These regions of chimpanzee ICAM-1 display even more striking K_{A}/K_{S} ratios (Table 3) than are seen for the whole protein comparisons, thus suggesting that the ICAM-1/LFA-1 interaction has been subjected to unusually strong selective pressures.

TABLE 3
K_{A}/K_{S} Ratios: Domains D1 + D2 of ICAM-1

| Species Compared | K_{A}/K_{S} Ratio |
|---|---|
| Chimpanzee to Human | 3.1 (P < 0.01) |
| Chimpanzee to Gorilla | 2.5 (P < 0.05) |
| Chimpanzee to Orangutan | 1.5 (P < 0.05) |
| Human to Gorilla | 1.0 |
| Human to Orangutan | 0.90 |
| Gorilla to Orangutan | 1.0 |

### Example 5

### Characterization of ICAM-1, ICAM-2 and ICAM-3 Positively Selected Sequences

A sequence identified by the methods of this invention may be further tested and characterized by cell transfection experiments. For example, human cells in culture, when transfected with a chimpanzee polynucleotide identified by the methods described herein (such as ICAM-1 (or ICAM-2 or ICAM-3); see below), could be tested for reduced viral dissemination and/or propagation using standard assays in the art, and compared to control cells. Other indicia may also be measured, depending on the perceived or apparent functional nature of the polynucleotide/polypeptide to be tested. For example, in the case of ICAM-1 (or ICAM-2 or ICAM-3), syncytia formation may be measured and compared to control (untransfected) cells. This would test whether the resistance arises from prevention of syncytia formation in infected cells.

Cells which are useful in characterizing sequences identified by the methods of this invention and their effects on cell-to-cell infection by HIV-1 are human T-cell lines which are permissive for infection with HIV-1, including, e.g., H9 and HUT78 cell lines, which are available from the ATCC.

For cell transfection assays, ICAM-1 (or ICAM-2 or ICAM-3) cDNA (or any cDNA identified by the methods described herein) can be cloned into an appropriate expression vector. To obtain maximal expression, the cloned ICAM-1 (or ICAM-2 or ICAM-3) coding region is operably linked to a promoter which is active in human T cells, such as, for example, an IL-2 promoter. Alternatively, an ICAM-1 (or ICAM-2 or ICAM-3) cDNA can be placed under transcriptional control of a strong constitutive promoter, or an inducible promoter. Expression systems are well known in the art, as are methods for introducing an expression vector into cells. For example, an expression vector comprising an ICAM-1 (or ICAM-2 or ICAM-3) cDNA can be introduced into cells by DEAE-dextran or by electroporation, or any other known method. The cloned ICAM-1 (or ICAM-2 or ICAM-3) molecule is then expressed on the surface of the cell. Determination of whether an ICAM-1 (or ICAM-2 or ICAM-3) cDNA is expressed on the cell surface can be accomplished using antibody(ies) specific for ICAM-1 (or ICAM-2 or ICAM-3). In the case of chimpanzee ICAM-1 (or ICAM-2 or ICAM-3) expressed on the surface of human T cells, an antibody which distinguishes between chimpanzee and human ICAM-1 (or ICAM-2 or ICAM-3) can be used. This antibody can be labeled with a detectable label, such as a fluorescent dye. Cells expressing chimpanzee ICAM-1 (or ICAM-2 or ICAM-3) on their surfaces can be detected using fluorescence-activated cell sorting and the anti-ICAM-1 (or ICAM-2 or ICAM-3) antibody appropriately labeled, using well-established techniques.

Transfected human cells expressing chimpanzee ICAM-1 (or ICAM-2 or ICAM-3) on their cell surface can then be tested for syncytia formation, and/or for HIV replication, and/or for number of cells infected as an index of cell-to-cell infectivity. The chimpanzee ICAM-1 (or ICAM-2 or ICAM-3)-expressing cells can be infected with HIV-1 at an appropriate dose, for example tissue culture infectious dose 50, i.e., a dose which can infect 50% of the cells. Cells can be plated at a density of about 5×10⁵ cells/ml in appropriate tissue culture medium, and, after infection, monitored for syncytia formation, and/or viral replication, and/or number of infected cells in comparison to control, uninfected cells. Cells which have not been transfected with chimpanzee ICAM-1 (or ICAM-2 or ICAM-3) also serve as controls. Syncytia formation is generally observed in HIV-1-infected cells (which are not expressing chimpanzee ICAM-1 (or ICAM-2 or ICAM-3)) approximately 10 days post-infection.

To monitor HIV replication, cell supernatants can be assayed for the presence and amount of p24 antigen. Any assay method to detect p24 can be used, including, for example, an ELISA assay in which rabbit anti-p24 antibodies are used as capture antibody, biotinylated rabbit anti-p24 antibodies serve as detection antibody, and the assay is developed with avidin-horse radish peroxidase. To determine the number of infected cells, any known method, including indirect immunofluorescence methods, can be used. In indirect immunofluorescence methods, human HIV-positive serum can be used as a source of anti-HIV antibodies to bind to infected cells. The bound antibodies can be detected using FITC-conjugated anti-human IgG, the cells visualized by fluorescence microscopy and counted.

Another method for assessing the role of a molecule such as ICAM-1 (or ICAM-2 or ICAM-3) involves successive infection of cells with HIV. Human cell lines, preferably those that do not express endogenous ICAM (although cell lines that do express endogenous ICAM may also be used), are transfected with either human or chimpanzee ICAM B1 or B2 or B3. In one set of experiments, HIV is collected from the supernatant of HIV-infected human ICAM-1 (or ICAM-2 or ICAM-3)-expressing cells and used to infect chimpanzee ICAM-1 (or ICAM-2 or ICAM-3)-expressing cells or human ICAM-1 (or ICAM-2 or ICAM-3)-expressing cells. Initial infectivity, measured as described above, of both the chimpanzee ICAM-1 (or ICAM-2 or ICAM-3)—and the human ICAM-1 (or ICAM-2 or ICAM-3)-expressing cells would be expected to be high. After several rounds of replication, cell to cell infectivity would be expected to decrease in the chimpanzee ICAM-1 (or ICAM-2 or ICAM-3) expressing cells, if chimpanzee ICAM-1 (or ICAM-2 or ICAM-3) confers resistance. In a second set of experiments, HIV is collected from the supernatant of HIV-infected chimpanzee ICAM-1 (or ICAM-2 or ICAM-3)-expressing cells, and used to infect human ICAM-1 (or ICAM-2 or ICAM-3)-expressing cells. In this case, the initial infectivity would be expected to be much lower than in the first set of experiments, if ICAM-1 (or ICAM-2 or ICAM-3) is involved in susceptibility to HIV progression. After several rounds of replication, the cell to cell infectivity would be expected to increase.

The identified human sequences can be used in establishing a database of candidate human genes that may be involved in conferring, or contributing to, AIDS susceptibility or resistance. Moreover, the database not only provides an ordered collection of candidate genes, it also provides the precise molecular sequence differences that exist between human and an AIDS-resistant non-human primate (such as chimpanzee) and thus defines the changes that underlie the functional differences.

### Example 6

### Molecular Modeling of ICAM-1 and ICAM-3

Modeling of the three-dimensional structure of ICAM-1 and ICAM-3 has provided additional evidence for the role of these proteins in explaining chimpanzee resistance to AIDS progression.

In the case of ICAM-1, 5 of the 6 amino acid replacements that are unique to the chimpanzee lineage are immediately adjacent (i.e., physically touching) to those amino acids identified by mutagenic studies as critical to LFA-1 binding. These five amino acid replacements are human L 18 to chimp Q 18, human K29 to chimp D29, human P45 to chimp G45, human R49 to chimp W49, and human E171 to chimp Q171. This positioning cannot be predicted from the primary structure (i.e., the actual sequence of amino acids). None of the amino acid residues critical for binding has changed in the chimpanzee ICAM-1 protein.

Such positioning argues strongly that the chimpanzee ICAM-1 protein=s basic function is unchanged between humans and chimpanzees; however, evolution has wrought fine-tuned changes that may help confer upon chimpanzees their resistance to progression of AIDS. The nature of the amino acid replacements is being examined to allow exploitation of the three-dimensional structural information for developing agents for therapeutic intervention. Strikingly, 4 of the 5 chimpanzee residues are adjacent to critical binding residues that have been identified as N-linked glycosylation sites. This suggests that differences exist in binding constants (to LFA-1) for human and chimpanzee ICAM-1. These binding constants are being determined. Should the binding constants prove lower in chimpanzee ICAM-1, it is possible to devise small molecule agents to mimic (by way of steric hindrance) the change in binding constants as a potential therapeutic strategy for HIV-infected humans. Similarly, stronger binding constants, if observed for chimpanzee ICAM-1, will suggest alternative strategies for developing therapeutic interventions for HIV-1 infected humans.

In the case of ICAM-3, a critical amino acid residue replacement from proline

(observed in seven humans) to glutamine (observed in three chimpanzees) is predicted from our modeling studies to significantly change the positional angle between domains 2 and 3 of human and chimpanzee ICAM-3. The human protein displays an acute angle at this juncture. Klickstein, et al., 1996 J. Biol. Chem. 27:239 20-27. Loss of this sharp angle (bend) is predicted to render chimpanzee ICAM-3 less easily packaged into HIV-1 virions (In infected humans, after ICAMs are packaged into HIV virions, cell-to-cell infectivity dramatically increases. Barbeau, B. et al., 1998 J. Virol. 72:7125-7136). This failure to easily package chimp ICAM-3 into HIV virions could then prevent the increase in cell-to-cell infectivity seen in infected humans. This would then account for chimpanzee resistance to AIDS progression.

A small molecule therapeutic intervention whereby binding of a suitably-designed small molecule to the human proline residue causes (as a result of steric hindrance) the human ICAM-1 protein to mimic the larger (i.e., less-acute) angle of chimpanzee ICAM-3 is possible. Conservation between the 2 proteins of the critical binding residues (and the general resemblance of immune responses between humans and chimpanzees) argues that alteration of this angle will not compromise the basic function of human ICAM-3. However, the human ICAM-3 protein would be rendered resistant to packaging into HIV virions, thus mimicking (in HIV-1 infected humans) the postulated pathway by which infected chimpanzees resist progression to AIDS.

Essentially the same procedures were used to identify positively selected chimpanzee ICAM-2 and ICAM-3 (see Table 4). The ligand binding domain of ICAM-1 has been localized as exhibiting especially striking positive selection in contrast to ICAMs-2 and -3, for which positive selection resulted in amino acid replacements throughout the protein. Thus, this comparative genomic analysis reveals that positive selection on ICAMs in chimpanzees has altered the proteins=primary structure, for example, in important binding domains. These alterations may have conferred resistance to AIDS progression in chimpanzees.

TABLE 4
K_{A}/K_{S} Ratios: ICAM-2 and 3 Whole Protein Comparisons

| Species Compared | K_{A}/K_{S} Ratio |
|---|---|
| Chimpanzee to Human ICAM-2 | 2.1 (P < 0.01) |
| Chimpanzee to Human ICAM-3 | 3.7 (P < 0.01) |

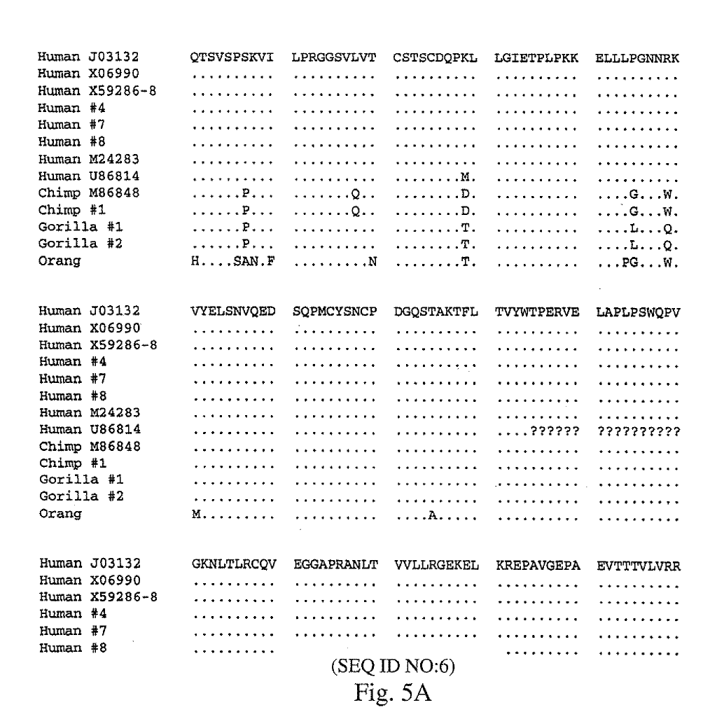

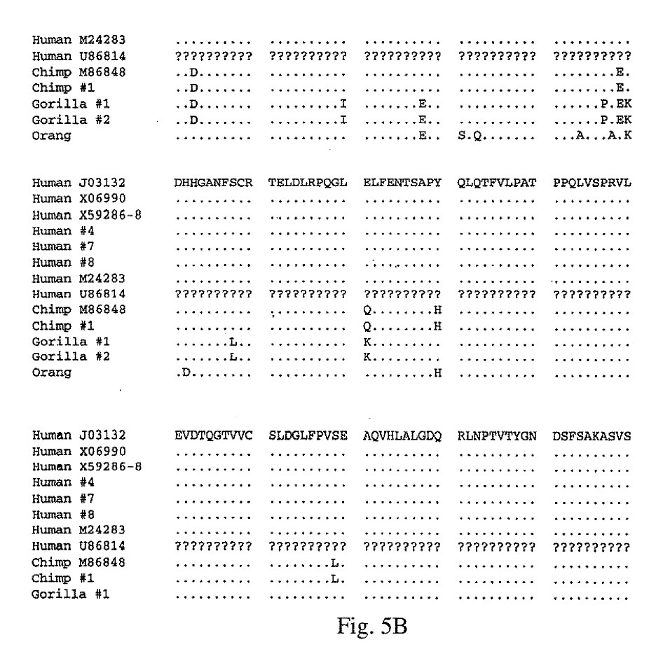

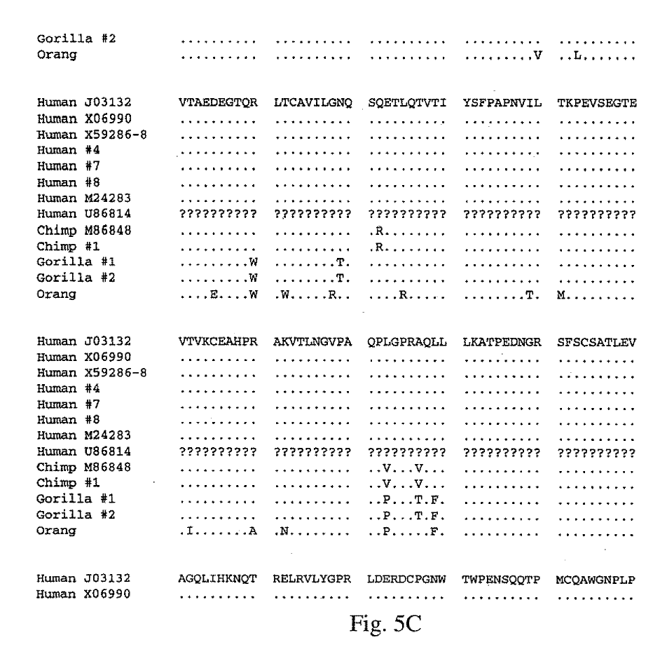

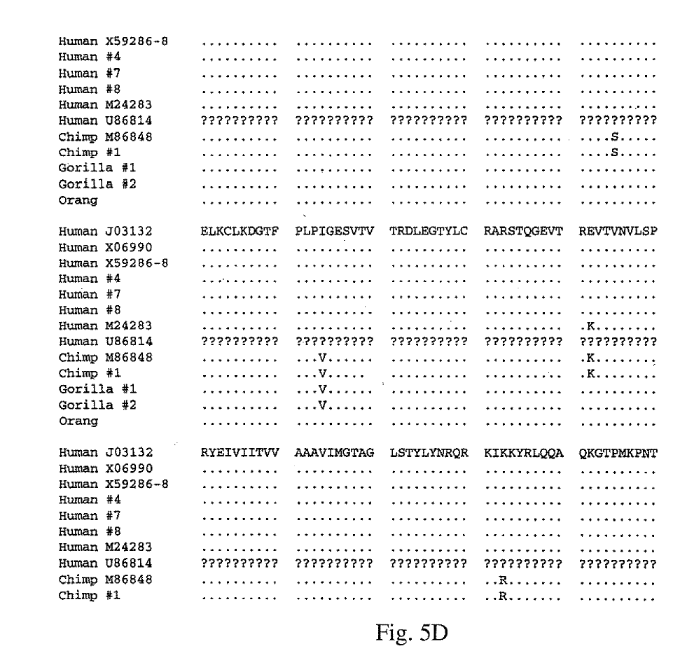

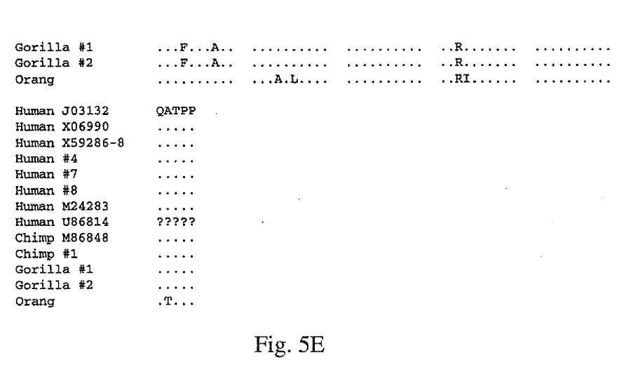

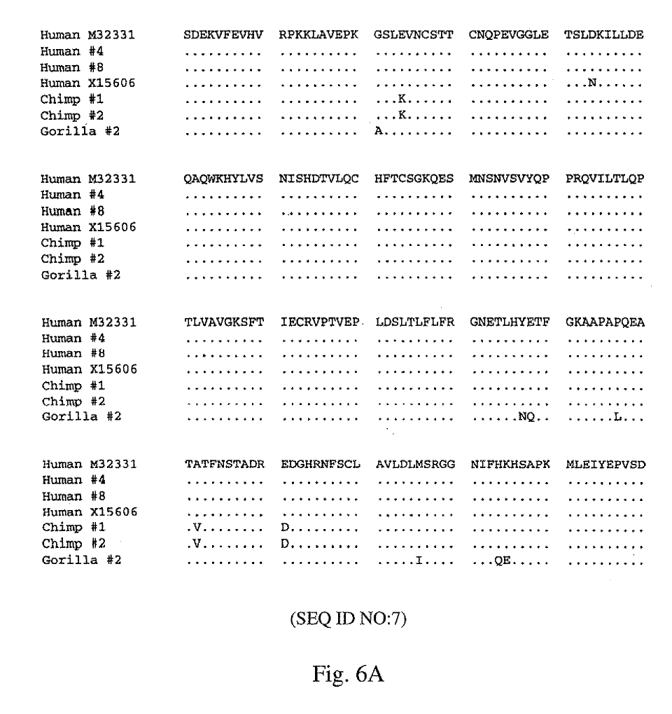

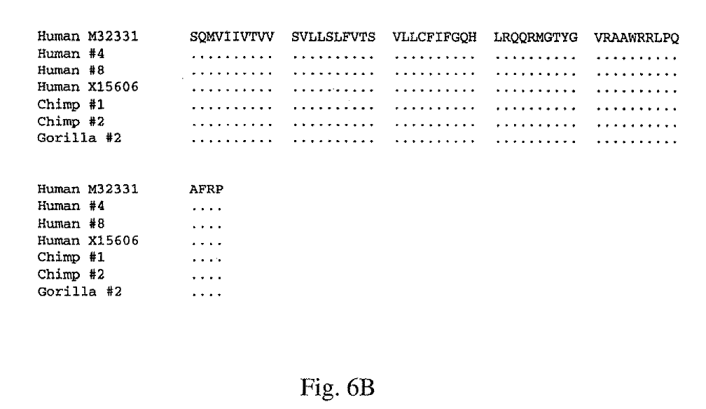

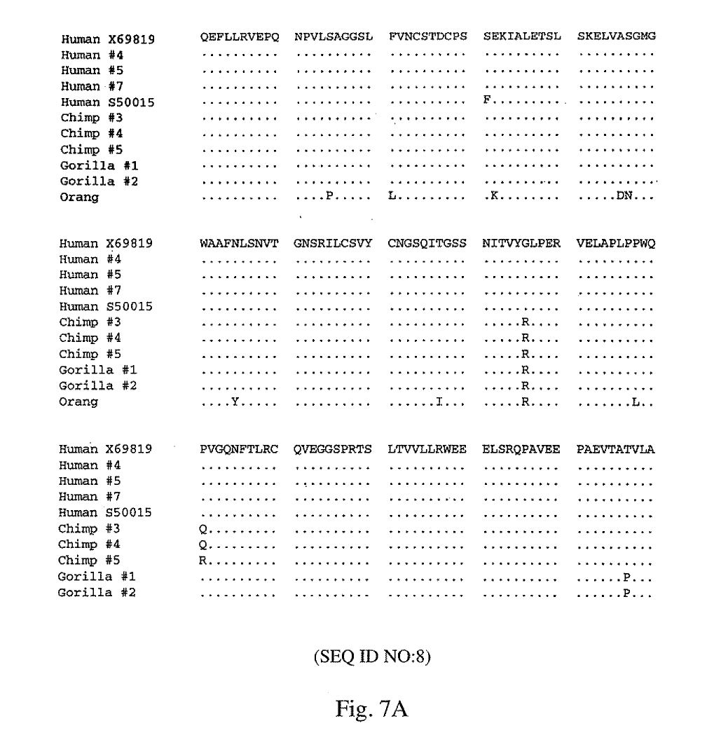

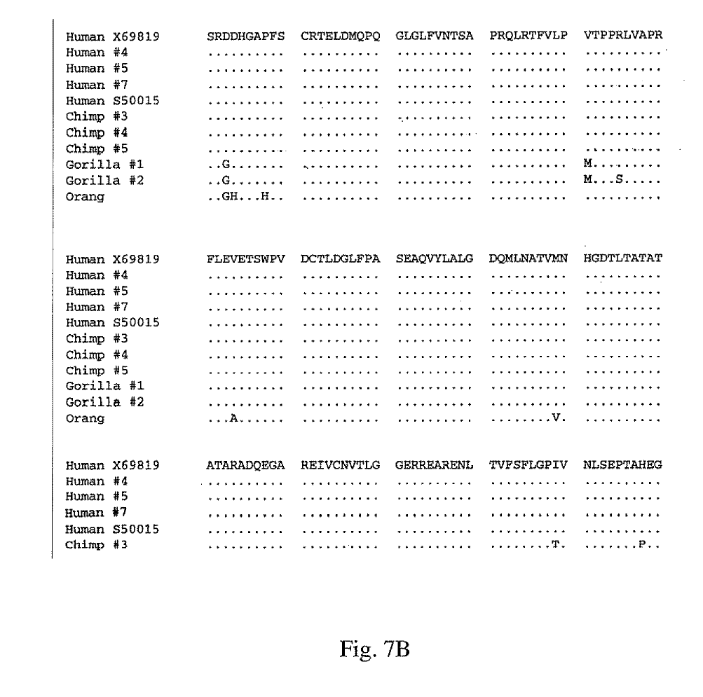

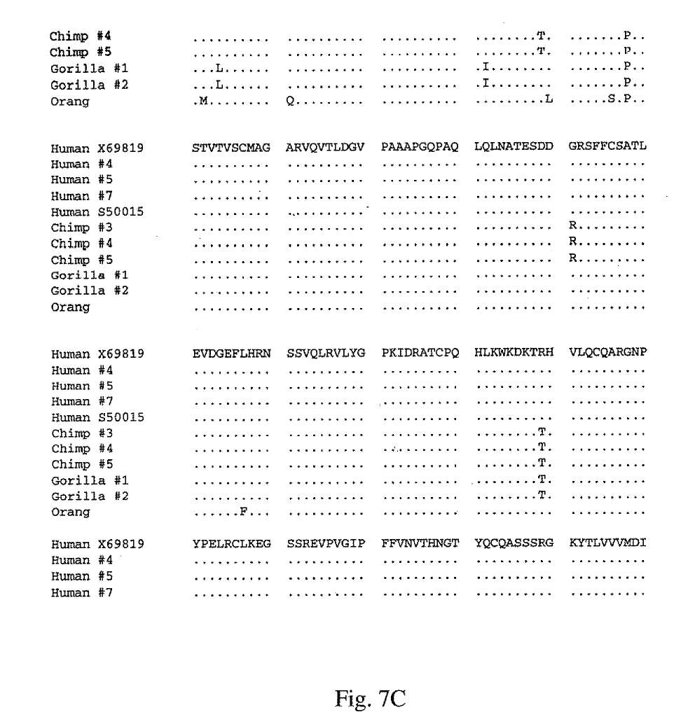

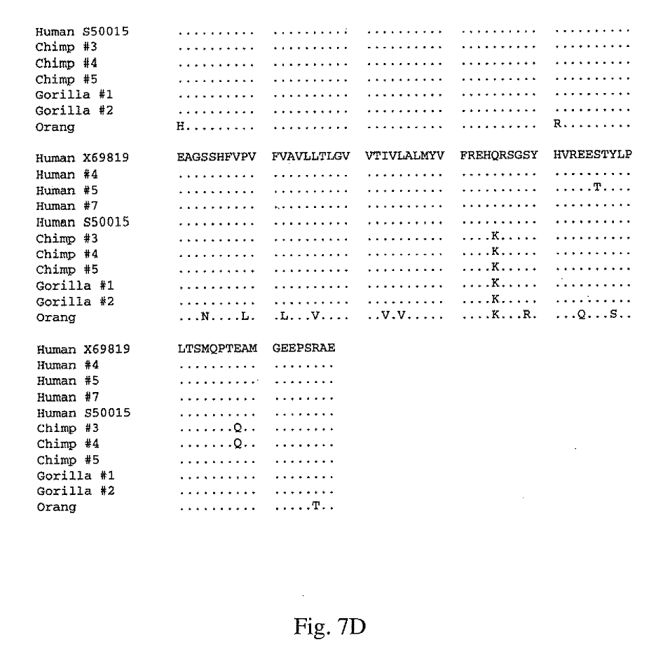

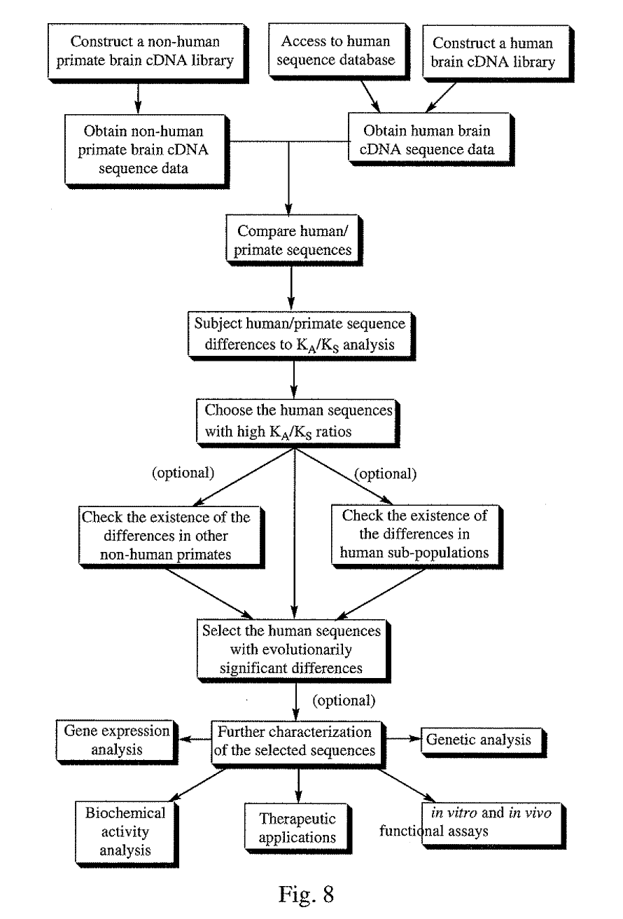

Binding of ICAM-1, -2, and -3 has been demonstrated to play an essential role in the formation of syncytia (i.e., giant, multi-nucleated cells) in HIV-infected cells in vitro. Pantaleo et al. (1991) J. Ex. Med. 173:511-514. Syncytia formation is followed by the depletion of CD⁺ cells in vitro. Pantaleo et al. (1991); Levy (1993) Microbiol. Rev. 57:183-189; Butini et al. (1994) Eur. J. Immunol. 24:2191-2195; Finkel and Banda (1994) Curr. Opin. Immunol. 6:605-615. Although syncytia formation is difficult to detect in vivo, clusters of infected cells are seen in lymph nodes of infected individuals. Pantaleo et al., (1993) N. Eng. J. Med. 328:327-335; Finkel and Banda (1994); Embretson et al. (1993) Nature 362:359-362; Pantaleo et al. (1993) Nature 362:355-358. Syncytia may simply be scavenged from the body too quickly to be detected. Fouchier et al. (1996) Virology 219:87-95. Syncytia-mediated loss of CD4⁺ cells in vivo has been speculated to occur; this could contribute directly to compromise of the immune system, leading to opportunistic infection and full-blown AIDS. Sodrosky et al. (1986) Nature 322:470-474; Hildreth and Orentas (1989) Science 244:1075-1078; Finkel and Banda (1994). Thus critical changes in chimpanzee ICAM-1, ICAM-2 or ICAM-3 may deter syncytia formation in chimpanzee and help explain chimpanzee resistance to AIDS progression. Because of the polyfunctional nature of ICAMs, these positively selected changes in the ICAM genes may additionally confer resistance to other infectious diseases or may play a role in other inflammatory processes that may also be of value in the development of human therapeutics. The polypeptide sequence alignments of ICAM-1, -2, and -3 are shown in FIGS. 5, 6, and 7, respectively.

### Example 6(A)

### Chimpanzee ICAM-1 Confers Immunoresistance to HIV and SIV

In the wild, chimpanzees maintain high viral loads of simian immunodeficiency virus 1 and 2 (SIV1/2), but never progress to immunocompromise. As the Intracellular Adhesion Molecule-1 (ICAM-1) molecule has been implicated in promoting the infectivity of HIV (the human analogue of SIV) in vivo, we chose to investigate this by molecular evolution analysis, looking for evidence of molecular-level Darwinian positive selection in the Catarrhine primates. We conducted pairwise comparisons of ICAM nucleotide sequences using a Ka/Ks approach. Ka/Ks ratios of human and chimpanzee ICAM-1 demonstrated that the chimpanzee ICAM-1 protein has been subjected to strong positive selection. We hypothesize that this selective episode resulted in chimpanzee resistance to immunosuppression. Molecular modeling of ICAM-1 crystal structures suggests that replacement of critical amino acid residues in chimpanzee ICAM-1 affect a site on the extracellular domain of ICAM-1 where a second ICAM-1 molecule binds to form a homodimer.

This altered dimer binding likely affects downstream activation of the cell. Absent an inflammatory stimulus, chimp cells may be able to tolerate SIV instead of progressing to cell death and immunocompromise. To study this further, we developed a model using human promonocytic cells co-cultured with an actively infected HIV cell line, the ACH2 line.

U937 promonocytic cells were transfected with chimp ICAM-1 using a CMV promoter and these chimp ICAM-1 expressing cells were cloned. The U937 cells were then placed into culture with ACH2 cells in the presence of lipopolysaccharide. Remarkably, co-cultures of the U937 chimp-ICAM-1 cells with ACH2 cells exhibited a decrease of up to 48% in the production of p24 after stimulation with LPS (p<0.05). To confirm that this was not a result unique to our population of U937 cells, THP1 promonocytic cells were also transfected with chimp ICAM-1. Under a similar experimental set up, co-cultures of chimp ICAM-1-transfected THP1 cells produced 38% less p24 than control THP1 co-cultures.

In current experiments, we find that the chimp ICAM-1 molecule, due to its altered dimerization binding site, leads to an anti-inflammatory milieu in which SIV/HIV is less able to cause cell injury via our work with site-directed mutagenesis.

### Example 6(B)

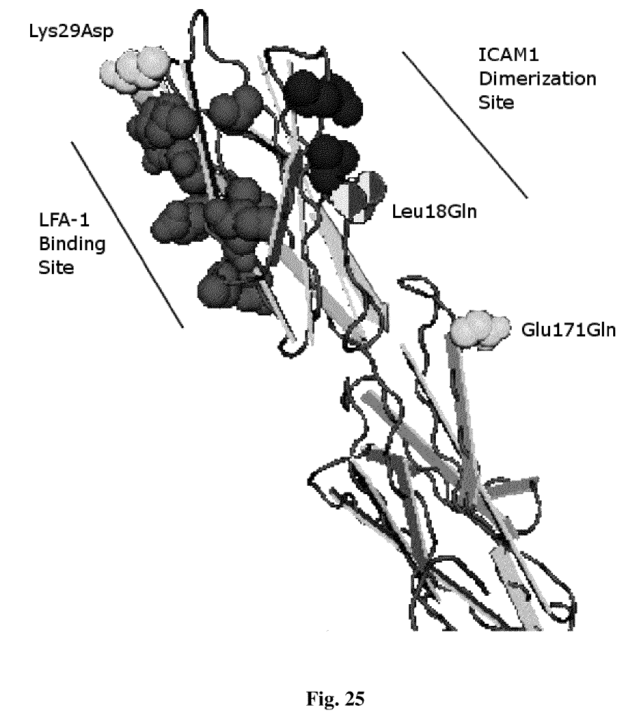

Using methods described more fully elsewhere herein, we found previously that chimpanzee ICAM-1 is positively selected. We determined a Ka/Ks ratio of 2.2 when chimpanzee ICAM-1 is compared to human ICAM-1 (Walter, et al 2005) (Ka/Ks ratios >0 indicate positive selection). We also determined the location of the amino acid replacements in chimpanzee ICAM-1 using published human ICAM-1 crystal structures (Walter, et al 2005).). It can be seen that the LFA-1 binding and the ICAM-1 dimerization surfaces are located on opposite faces of domain 1, and that the chimpanzee amino acid replacements are located exclusively in a hydrophobic plane in ICAM-1 domain 1; a plane predicted by others to be important in homodimerization of ICAM-1 molecules (see FIG. 25).

We then tested the affect of ICAM-1 on HIV-1 infectivity in an in vitro model. The laboratory of our collaborator prepared human THP-1 macrophage cell lines transfected with chimpanzee ICAM-1 and CMV promoter for constitutive expression. Control cell lines were transfected with a mock plasmid. The THP-1 cells were co-cultured with ACH2 cells, a stable line of T cells that constitutively express HIV-1. The ACH2 cells were used because contact between T-cells and macrophages (or dendritic cells) is fundamental to HIV-1 infection. Experiments were repeated four times. ICAM-1 is upregulated under conditions such as inflammation, hypoxia, coagulation and infection. Routine infections in HIV-1 positive patients are associated with increased HIV-1 expression. Therefore, we cultured THP-1 cells with ACH2 in the presence of bacterial lipopolysaccharide (LPS) (100 ng/mL) in order to mimic inflammation and increase the expression of ICAM-1. HIV-1 production was measured using an immunoassay for p24 levels in culture supernatants.

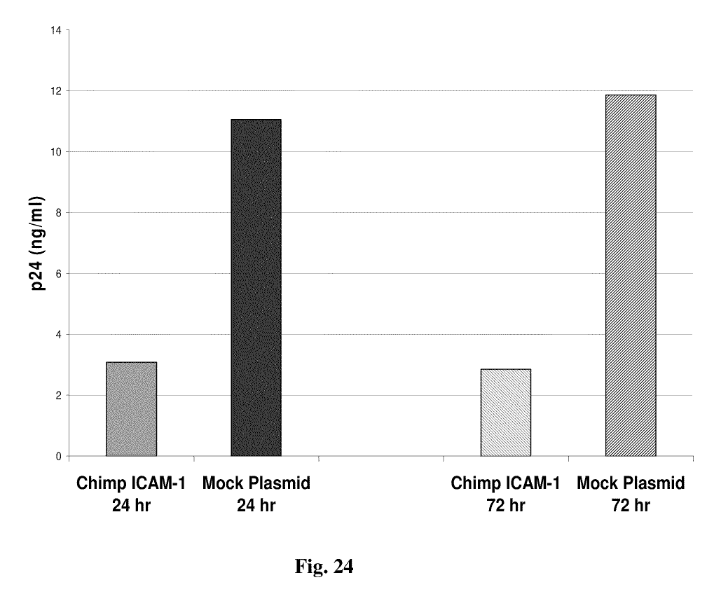

As shown in FIG. 24, co-culture of mock transfected THP-1 cells plus ACH2 cells in the presence of LPS induced HIV-1 expression, while co-culture of chimpanzee ICAM-1-transfected THP-1 cells plus ACH2 in the presence of LPS yielded less HIV-1 production, both at 24 hours (approximately 72% reduction in HIV-1 production) and 72 hours (approximately 76% reduction in HIV-1 production). The results represent the mean of duplicate measurements. It should be noted that the endogenous human ICAM-1 was also expressed by the THP-1 cells, thus, it appears that the mechanism of chimpanzee ICAM-1 HIV-1 suppression is active even in the presence of human ICAM-1. The experiments were repeated using U937 macrophage cells, with similar reductions in virus production in the presence of chimpanzee ICAM-1.

These data show that ICAM-1 plays a role in the mechanism of chimpanzee resistance to disease progression.

### Example 6(C)

### Identifying Modulators of ICAM-1 Function

Humans and our closest living relatives, the chimpanzees, share genomes with high degrees of similarity. However, conspicuous differences exist in how these species respond to a few pathogens, most notably, HIV-1. It has long been recognized that common chimpanzees (Pan troglodytes), although occasionally infected by SIV and susceptible to infection by HIV-1, are resistant to progressive immunosuppression (i.e., “AIDS”. The demonstration that SIVcpz (the progenitor of HIV-1) originated in chimpanzees suggests that their resistance may stem from evolutionary accommodation by ancestral chimpanzees to infection by this CD4 tropic lentivirus. If proteins responsible for chimpanzee AIDS resistance could be identified and the specific adaptive changes in such proteins identified, then small molecule therapeutics could be devised that interact with human homologs of adapted chimpanzee proteins to mimic (in human patients) the mechanisms by which chimpanzee proteins modulate resistance to progression.

Knowledge of the details of by which HIV-1-infected chimpanzees are rendered refractory to progressive immunosuppression can assist in developing novel therapeutics for HIV-1-infected patients. A chimpanzee protein identified as positively selected in chimpanzees compared to humans, Intracellular Adhesion Molecule-1 (ICAM-1), significantly reduces HIV production by infected cells in culture.

Clearly, chimpanzee resistance to progression to full-blown AIDS must result from evolutionary responses of the chimpanzee immune system to the strong selective pressure that resulted from introduction of the ancestral virus to chimpanzee populations. The close similarity of chimpanzee and human immune systems is unsurprising, since humans and chimpanzees share a very recent common ancestor (only 5-8 million years). Because of the strong patterns of evolutionary conservation observed for the vast majority of homologous human and chimpanzee genes, our positive selection-based data-mining approach is effective and powerful in narrowing the search for genes important in conferring a survival advantage, such as those underlying chimpanzee resistance to AIDS.

### Example 6(D)

### Transfection of Human U1 Cell Lines to Express an Adapted Chimpanzee Gene and Determination of Differential Rates of Viral Infectivity

We chose Intracellular Adhesion Molecule-1 (ICAM-1) to examine in vitro because it had been shown to be:

Upregulated in cells infected with HIV-1

Selectively incorporated into the HIV-1 coat

Important in cell-virus interaction

Positively selected (adaptively evolved) in chimpanzees

Creation of stable cell lines expressing chimpanzee ICAM-1 (chICAM-1).

The cDNA for chICAM-1 was inserted into the plasmid pCAG containing a neomycin and also a puromycin (pBABE.puro) resistance cassette. Control (mock) without the chICAM-1 was also constructed.

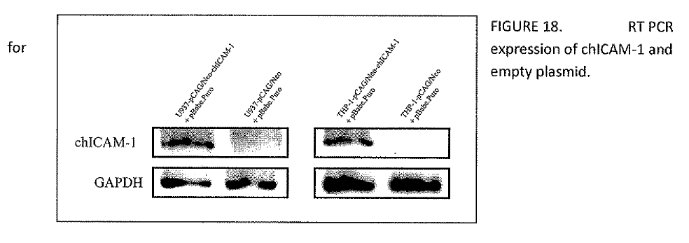

These plasmids contain a CMV promoter for constitutive expression. Human THP-1 as well as U937 macrophage cell lines were transfected with the plasmids using lipofectamine. After the transfection, the cells were expanded in 10% Fetal Calf Serum (FCS) in the presence of neomycin and puromycin. Limiting dilutions were used to select clones. As shown in FIG. 18, constitutive steady-state expression of chICAM-1 was expressed in both clones containing the chICAM-1. Using the chICAM-1 specific primers (that is primers that do not recognize human ICAM-1) there was no expression in mock-transfected THP-1 or mock-transfected U937 cells.

The effect of co-culture of chICAM-1 THP-1 expressing cells with ACH2 cells.

ACH2 cells are a stable line of HIV-1 expressing T-cells. ACH2 cells express HIV-1 constitutively. Macrophage (or dendritic cell) contact with T-cells is fundamental to HIV-1 infection. Therefore, we co-cultured the macrophage cell line THP-1 expressing chICAM-1 (CS) with human ACH2 cells at a concentration of 8×10⁵ cells (4×10⁵ THP-1 and 4×10⁵ ACH2) in

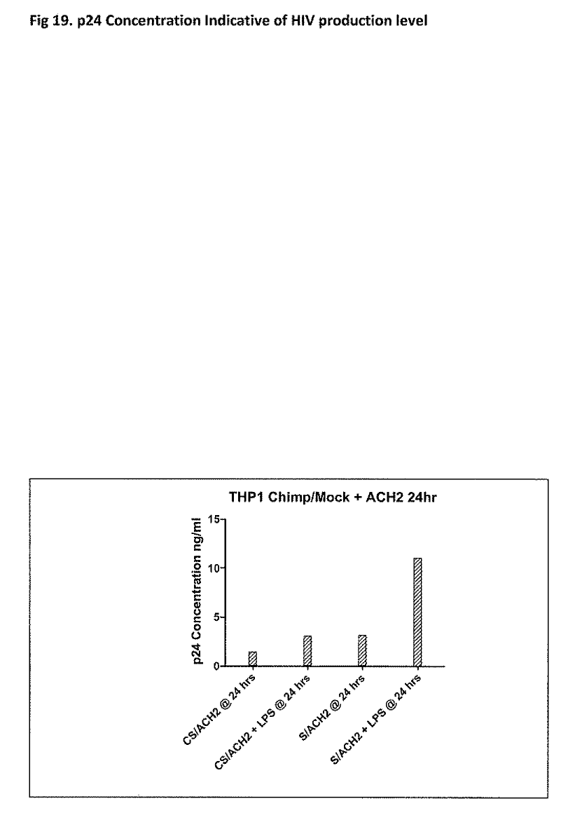

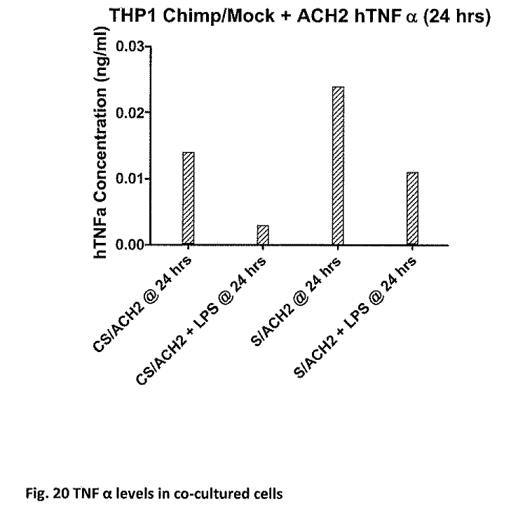

1.0 ml of RPMI plus 10% FCS. As shown in FIG. 19, co-culture of mock transfected THP-1 (S) plus ACH2 cells induced HIV-1 expression as measured by an immunoassay for p24 in the cell supernatants. By comparison, co-culture of chICAM-1 transfected THP-1 plus ACH2 yielded less HIV-1 production (55% reduction). At 24 hours, there was also a reduction (35%) in production of the cytokine, tumor necrosis factor (TNF α), in these co-cultures (see below), FIG. 20.

ICAM-1 is upregulated under conditions such as inflammation, hypoxia, coagulation and infection. Routine infections in HIV-1 positive patients are associated with increased HIV-1 expression. Therefore, we cultured THP-1 cells with ACH2 in the presence of bacterial lipopolysaccharide (LPS) in order to mimic inflammation and increase the expression of chICAM-1.

As shown (FIG. 20), there was a marked increase in p24 in mock transfected THP-1 cells (12.5 ng/mL). In contrast, LPS-stimulation of the co-culture of THP-1 cells expressing produced considerably less p24 after a 24 hour incubation (2.6 ng/mL). The effect of the LPS-induced differences is most likely due to the stimulating effect of LPS on ICAM-1 expression in the THP-1 since LPS has no significant effect on ACH2 cells. Although the levels of TNF a were markedly lower with LPS-stimulation, THP-1 expressing chICAM-1 was still lower (FIG. 20).

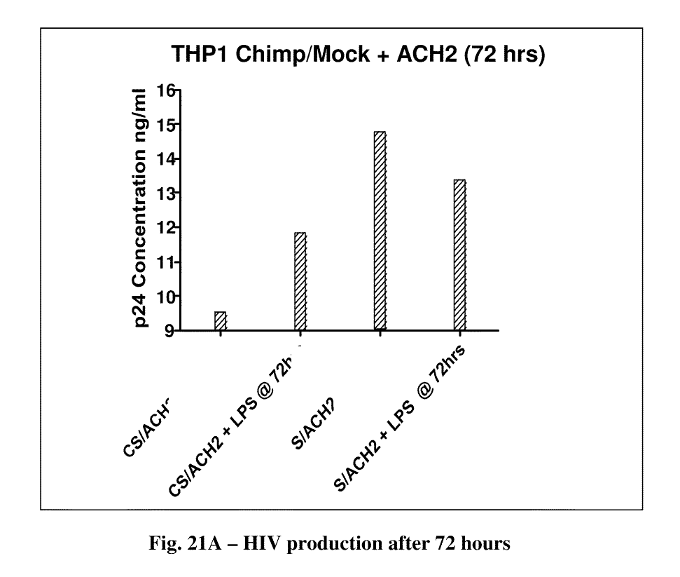

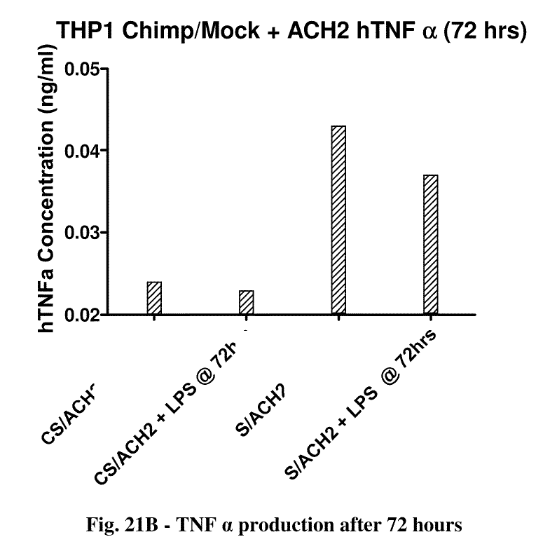

4c. The effect of co-cultures of chICAM-1 THP-1 cells with ACH2 cells after 72 hours. As shown on (FIG. 21), ACH2 cells when co-cultured with THP-1 cells expressing chICAM-1 (CS) produced approximately 80% less p24 when compared to ACH2 cells co-cultured with mock-transfected cells (S). The results represent the mean of duplicate measurements after 72 hours in culture. In addition, the co-culture was incubated for 72 hours in the presence of LPS (100 ng/mL). Under these conditions, there was clearly less p24 in produced by the ACH2 cells when incubated with chICAM-1 compared to mock-transfected (see FIG. 4 below, left panel) Levels of TNF a were also markedly lower in THP-1 cells expressing chICAM-1 (CS) compared to mock-transfected cells (S) at 72 hours whether with or without LPS (see FIG. 21, right panel)

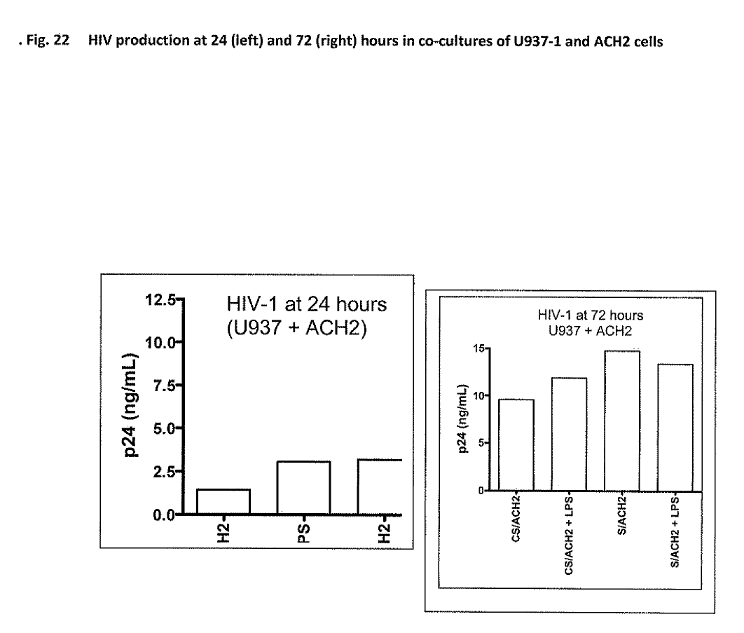

Effect of co-culture of U937-1 with ACH2 cells on p24 levels. We next examined the effect of U937 cells expressing chICAM-1 (CS). Similar to THP-1 cells expressing chICAM-1, we again observed a reduction in HIV-1 expression. As shown below, ACH2 cells when co-cultured with U937 cells expressing chICAM-1 (CS) produced approximately 50% less p24 when compared to ACH2 cells co-cultured with mock-transfected cells (S). The results represent the mean of duplicate measurements after 24 and 72 hours in culture. In addition, the co-culture was incubated for 24 or 72 hours in the presence of LPS (100 ng/mL) at a cellular concentration of 1.0×10⁶ cells (5×10⁵ U937 plus 5×10⁵ ACH2) per 1.0 ml of medium, RPMI plus 10% FCS. Under these conditions, there was more p24 in produced by the ACH2 cells with mock-transfected cells compared to chICAM-1 expressing cells. See FIG. 22.

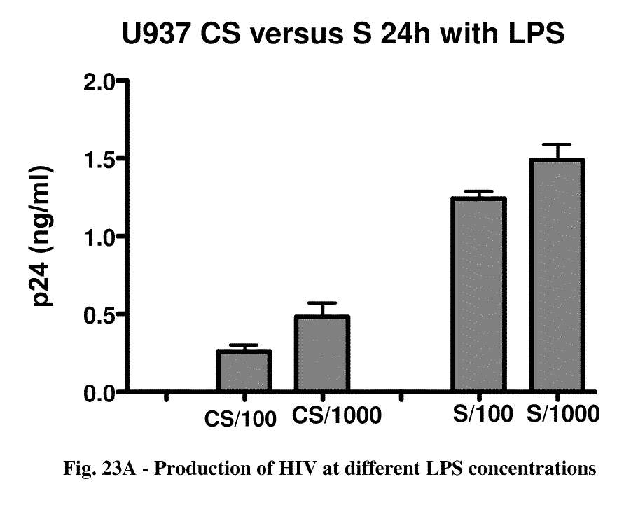

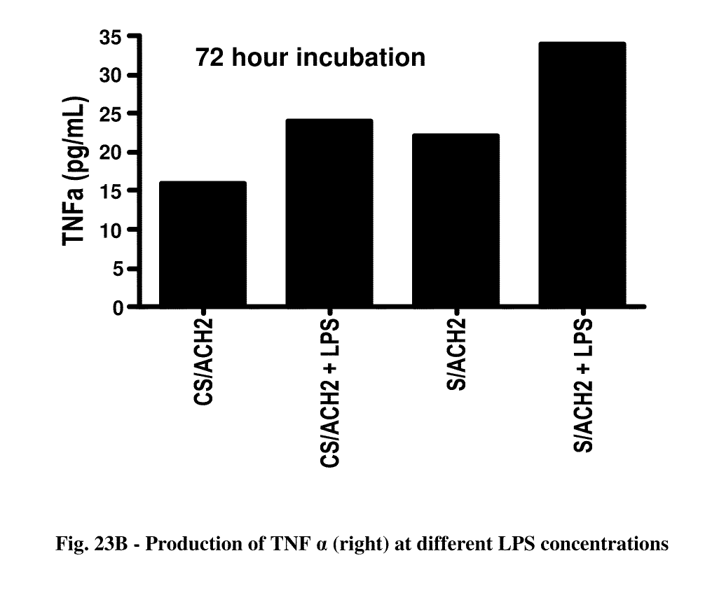

Effect of increasing the concentration of LPS in co-cultures of U937 cells with ACH2 cells on HIV-1 production. We next repeated the study of U937 cells stimulated with two different concentrations of LPS, 100 and 1000 ng/mL. As shown (FIG. 23), there was decrease in the production of HIV-1 production under both conditions at 24 hours in co-cultures of chICAM-1 expressing cells (CS) compared to mock-transfected cells (S). For example, p24 levels in the mock-transfected cells stimulated with 100 ng/mL of LPS was 1.29 ng/mL but in chICAM-1 expressing cells also stimulated with 100 ng/mL was 0.29 ng/mL, a decrease of nearly 80%. When U937 cells transfected with the empty plasmid were stimulated with 1000 ng/mL, the production increased from 1.29 ng/mL to 1.59 ng/mL but in U937 cells expressing chICAM-1, the level of p24 was 0.49 ng/mL. In these cultures TNF a was also measured (see FIG. 23 right panel).

Thus, the lower production of p24 in co-cultures U937 cells expressing chICAM-1 is a highly consistent finding and is independent of the amount of LPS stimulation. These results, in which chimpanzee ICAM-1 suppresses production of the HIV-1 virus in infected cells, are powerful evidence that this protein explains how HIV-1-infected chimpanzees resist progression to AIDS.

The ultimate commercial application of the proposed research is to identify small molecule compounds that mimic chimpanzee disease resistance mechanisms that could be developed as human drugs. As mentioned above, in the case of AIDS drugs, such therapeutics are expected to have fewer side effects so that patient use and follow-through will be greater, and be more lastingly effective, because they target stable host proteins instead of mutating viral proteins. One important societal impact of the work is to have a better treatment for AIDS so that AIDS patients can lead longer, productive lives.

Importantly, the positively selected genes identified appear to be part of a general immune response to RNA virus infections. The result could lead to therapeutics to treat infections by other RNA viruses, such as hepatitis C. Evolutionary studies indicate that the parent strain for the various hepatitis C strains isolated from humans worldwide originated in Africa, and is likely to have come from chimpanzees. Approximately four million Americans are infected with the hepatitis C virus (HCV), and worldwide the number approaches 40 million. HCV infection can lead to hepatocellular carcinoma, which is nearly always fatal and kills 14,500 Americans each year. Thus identification of drugs that can ameliorate the effects of chronic infection are valuable both from a societal and commercial viewpoint. Chronic hepatitis C infection is much less severe in chimpanzees than in humans. Like HIV infection, this difference is likely due to differences in key host proteins. Because four chimpanzee proteins EG scientists identified as positively selected become active upon infection with several different RNA viruses, including HIV and hepatitis C, compounds that EG identifies that interact with these proteins and block HIV infectivity will also be evaluated for applicability to prevent or treat hepatitis C and other RNA viral infections.

### Example 7

### Identifying Positive Selection of MIP-1a

MIP-1α is a chemokine that has been shown to suppress HIV-1 replication in human cells in vitro (Cocchi, F. et al., 1995 Science 270:1811-1815). The chimpanzee homologue of the human MIP-1β gene was PCR-amplified and sequenced. Calculation of the K_{A}/K_{S} ratio (2.1, P<0.05) and comparison to the gorilla homologue reveals that the chimpanzee gene has been positively-selected. As for the other genes discussed herein, the nature of the chimpanzee amino acid replacements is being examined to determine how to exploit the chimpanzee protein for therapeutic intervention.

### Example 8

### Identifying Positive Selection of 17-β-Hydroxysteroid Dehydrogenase

Using the methods of the present invention, a chimpanzee gene expressed in brain has been positively-selected (K_{A}/K_{S}=1.6) as compared to its human homologue (GenBank Acc. # X87176) has been identified. The human gene, 17-P hydroxysteroid dehydrogenase type IV, codes for a protein known to degrade the two most potent estrogens, β-estradiol, and 5-diol (Adamski, J. et al. 1995 Biochem J. 311:437-443). Estrogen-related cancers (including, for example, breast and prostate cancers) account for some 40% of human cancers. Interestingly, reports in the literature suggest that chimpanzees are resistant to tumorigenesis, especially those that are estrogen-related. This protein may have been positively-selected in chimpanzees to allow more efficient degradation of estrogens, thus conferring upon chimpanzees resistance to such cancers. If so, the specific amino acid replacements observed in the chimpanzee protein may supply important information for therapeutic intervention in human cancers.

### Example 9

### cdNA Library Construction for Chimpanzee Brain Tissue

A chimpanzee brain cDNA library is constructed using chimpanzee brain tissue. The chimpanzee brain tissue can be obtained after natural death so that no killing of an animal is necessary for this study. In order to increase the chance of obtaining intact mRNAs expressed in brain, however, the brain is obtained as soon as possible after the animal=s death. Preferably, the weight and age of the animal are determined prior to death. The brain tissue used for constructing a cDNA library is preferably the whole brain in order to maximize the inclusion of mRNA expressed in the entire brain. Brain tissue is dissected from the animal following standard surgical procedures.

Total RNA is extracted from the brain tissue and the integrity and purity of the RNA are determined according to conventional molecular cloning methods. Poly A+ RNA is selected and used as template for the reverse-transcription of cDNA with oligo (dT) as a primer. The synthesized cDNA is treated and modified for cloning using commercially available kits. Recombinants are then packaged and propagated in a host cell line. Portions of the packaging mixes are amplified and the remainder retained prior to amplification. The library can be normalized and the numbers of independent recombinants in the library is determined.

### Example 10

### Sequence Comparison of Chimpanzee and Human Brain cDNA

Randomly selected chimpanzee brain cDNA clones from the cDNA library are sequenced using an automated sequencer, such as the ABI 377. Commonly used primers on the cloning vector such as the M13 Universal and Reverse primers are used to carry out the sequencing. For inserts that are not completely sequenced by end sequencing, dye-labeled terminators are used to fill in remaining gaps.

The resulting chimpanzee sequences are compared to human sequences via database searches, e.g., BLAST searches. The high scoring “hits,” i.e., sequences that show a significant (e.g., >80%) similarity after BLAST analysis, are retrieved and analyzed. The two homologous sequences are then aligned using the alignment program CLUSTAL V developed by Higgins et al. Any sequence divergence, including nucleotide substitution, insertion and deletion, can be detected and recorded by the alignment.

The detected sequence differences are initially checked for accuracy by finding the points where there are differences between the chimpanzee and human sequences; checking the sequence fluorogram (chromatogram) to determine if the bases that appear unique to human correspond to strong, clear signals specific for the called base; checking the human hits to see if there is more than one human sequence that corresponds to a sequence change; and other methods known in the art as needed. Multiple human sequence entries for the same gene that have the same nucleotide at a position where there is a different chimpanzee nucleotide provides independent support that the human sequence is accurate, and that the chimpanzee/human difference is real. Such changes are examined using public database information and the genetic code to determine whether these DNA sequence changes result in a change in the amino acid sequence of the encoded protein. The sequences can also be examined by direct sequencing of the encoded protein.

### Example 11

### Molecular Evolution Analysis of Human Brain Sequences Relative to Other Primates

The chimpanzee and human sequences under comparison are subjected to K_{A}/K_{S} analysis. In this analysis, publicly available computer programs, such as Li 93 and INA, are used to determine the number of non-synonymous changes per site (K_{A}) divided by the number of synonymous changes per site (K_{S}) for each sequence under study as described above. This ratio, K_{A}/K_{S}, has been shown to be a reflection of the degree to which adaptive evolution, i.e., positive selection, has been at work in the sequence under study. Typically, full-length coding regions have been used in these comparative analyses. However, partial segments of a coding region can also be used effectively. The higher the K_{A}/K_{S} ratio, the more likely that a sequence has undergone adaptive evolution. Statistical significance of K_{A}/K_{S} values is determined using established statistic methods and available programs such as the t-test. Those genes showing statistically high K_{A}/K_{S} ratios between chimpanzee and human genes are very likely to have undergone adaptive evolution.

To further lend support to the significance of a high K_{A}/K_{S} ratio, the sequence under study can be compared in other non-human primates, e.g., gorilla, orangutan, bonobo. These comparisons allow further discrimination as to whether the adaptive evolutionary changes are unique to the human lineage compared to other non-human primates. The sequences can also be examined by direct sequencing of the gene of interest from representatives of several diverse human populations to assess to what degree the sequence is conserved in the human species.

### Example 12

### Further Sequence Characterization of Selected Human Brain Sequences

Human brain nucleotide sequences containing evolutionarily significant changes are further characterized in terms of their molecular and genetic properties, as well as their biological functions. The identified coding sequences are used as probes to perform in situ mRNA hybridization that reveals the expression pattern of the gene, either or both in terms of what tissues and cell types in which the sequences are expressed, and when they are expressed during the course of development or during the cell cycle. Sequences that are expressed in brain may be better candidates as being associated with important human brain functions. Moreover, the putative gene with the identified sequences are subjected to homologue searching in order to determine what functional classes the sequences belong to.

Furthermore, for some proteins, the identified human sequence changes may be useful in estimating the functional consequence of the change. By using such criteria a database of candidate genes can be generated. Candidates are ranked as to the likelihood that the gene is responsible for the unique or enhanced abilities found in the human brain compared to chimpanzee or other non-human primates, such as high capacity information processing, storage and retrieval capabilities, language abilities, as well as others. In this way, this approach provides a new strategy by which such genes can be identified. Lastly, the database not only provides an ordered collection of candidate genes, it also provides the precise molecular sequence differences that exist between human and chimpanzee (and other non-human primates), and thus defines the changes that underlie the functional differences.

In some cases functional differences are evaluated in suitable model systems, including, but not limited to, in vitro analysis such as indicia of long term potentiation (LTP), and use of transgenic animals or other suitable model systems. These will be immediately apparent to those skilled in the art.

### Example 13

### Identification of Positive Selection in a Human Tyrosine Kinase Gene

Using the methods of the present invention, a human gene (GenBank Acc.# AB014541), expressed in brain has been identified, that has been positively-selected as compared to its gorilla homologue. This gene, which codes for a tyrosine kinase, is homologous to a well-characterized mouse gene (GenBank Acc.# AF011908) whose gene product, called AATYK, is known to trigger apoptosis (Gaozza, E. et al. 1997 Oncogene 15:3127-3135). The literature suggests that this protein controls apoptosis in the developing mouse brain (thus, in effect, “sculpting” the developing brain). The AATYK-induced apoptosis that occurs during brain development has been demonstrated to be necessary for normal brain development.

There is increasing evidence that inappropriate apoptosis contributes to the pathology of human neurodegenerative diseases, including retinal degeneration, Huntington's disease, Alzheimer's disease, Parkinson's disease and spinal muscular atrophy, an inherited childhood motoneuron disease. On the other hand in neural tumour cells, such as neuroblastoma and medulloblastoma cells, apoptotic pathways may be disabled and the cells become resistant to chemotherapeutic drugs that kill cancer cells by inducing apoptosis. A further understanding of apoptosis pathways and the function of apoptosis genes should lead to a better understanding of these conditions and permit the use of AATYKI in diagnosis of such conditions.

Positively-selected human and chimpanzee AATYK may constitute another adaptive change that has implications for disease progression. Upon resolution of the three-dimensional structure of human and chimpanzee AATYK, it may be possible to design drugs to modulate the function of AATYK in a desired manner without disrupting any of the normal functions of human AATTK.

It has been demonstrated that mouse AATYK is an active, non-receptor, cytosolic kinase which induces neuronal differentiation in human adrenergic neuroblastoma (NB):SH-SY5Y cells. AATYK also promotes differentiation induced by other agents, including all-trans retinoic acid (RA), 12-O-Tetradecanoyl phorbol 13-acetate (TPA) and IGF-I. Raghunath, et al., Brain Res Mol Brain Res. (2000) 77:151-62. In experiments with rats, it was found that the AATYK protein was expressed in virtually all regions of the adult rat brain in which neurons are present, including olfactory bulb, forebrain, cortex, midbrain, cerebellum and pons. Immunohistochemical labeling of adult brain sections showed the highest levels of AATYK expression in the cerebellum and olfactory bulb. Expression of AATYK was also up-regulated as a function of retinoic acid-induced neuronal differentiation of p 19 embryonal carcinoma cells, supporting a role for this protein in mature neurons and neuronal differentiation. Baker, et al., Oncogene (2001) 20:1015-21.

Nicolini, et al., Anticancer Res (1998) 18:2477-81 showed that retinoic acid (RA) differentiated SH-SY5Y cells were a suitable and reliable model to test the neurotoxicity of chemotherapeutic drugs without the confusing effects of the neurotrophic factors commonly used to induce neuronal differentiation. The neurotoxic effect and the course of the changes is similar to that observed in clinical practice and in in vivo experimental models. Thus, the model is proposed as a screening method to test the neurotoxicity of chemotherapy drugs and the possible effect of neuroprotectant molecules and drugs. Similarly, AATYK differentiated SYSY-5Y cells could be used as a model for screening chemotherapeutic drugs and possible side effects of neuroprotectant molecules and drugs.

It has also been shown that AATYK mRNA is expressed in neurons throughout the adult mouse brain. AATYK possessed tyrosine kinase activity and was autophosphorylated when expressed in 293 cells. AATYK mRNA expression was rapidly induced in cultured mouse cerebellar granule cells during apoptosis induced by KCl. The number of apoptotic granule cells overexpressing wild-type AATYK protein was significantly greater than the number of apoptotic granule cells overexpressing a mutant AATYK that lacked tyrosine kinase activity. These findings suggest that through its tyrosine kinase activity, AATYK is also involved in the apoptosis of mature neurons. Tomomura, et al., Oncogene (2001) 20(9):1022-32.

The tyrosine kinase domain of AATYK protein is highly conserved between mouse, chimpanzee, and human (as are most tyrosine kinases). Interestingly, however, the region of the protein to which signaling proteins bind has been positively-selected in humans, but strongly conserved in both chimpanzees and mice. The region of the human protein to which signaling proteins bind has not only been positively-selected as a result of point nucleotide mutations, but additionally displays duplication of several src homology 2 (SH2) binding domains that exist only as single copies in mouse and chimpanzee. This suggests that a different set of signaling proteins may bind to the human protein, which could then trigger different pathways for apoptosis in the developing human brain compared to those in mice and chimpanzees. Such a gene thus may contribute to unique or enhanced human cognitive abilities. Human AATYK has been mapped on 25.3 region of chromosome 17. Seki, et al., J Hum Genet (1999) 44:141-2.

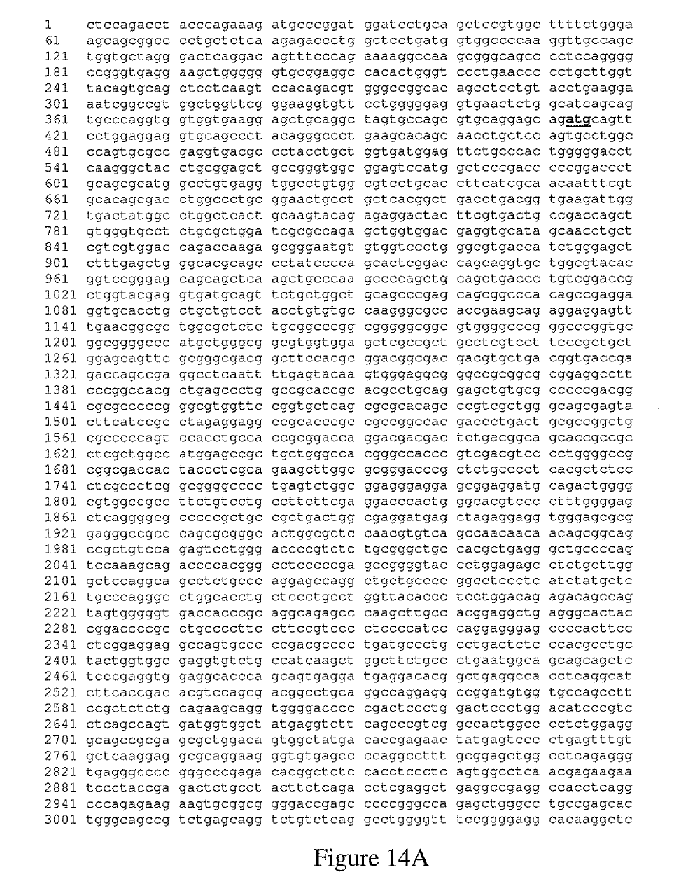

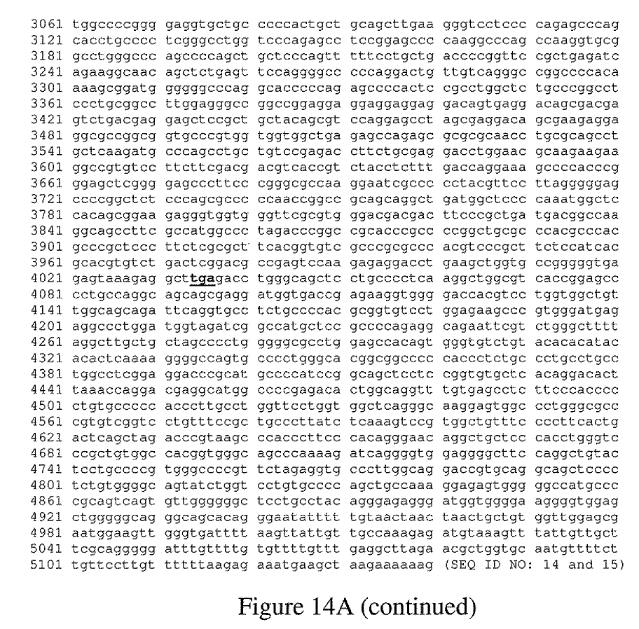

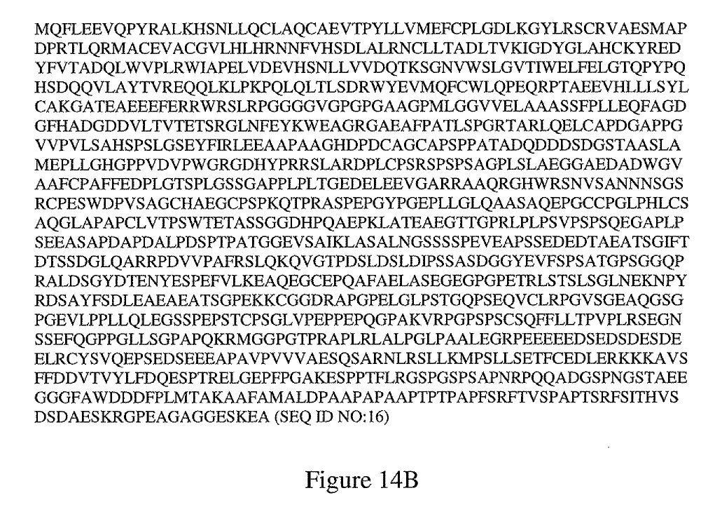

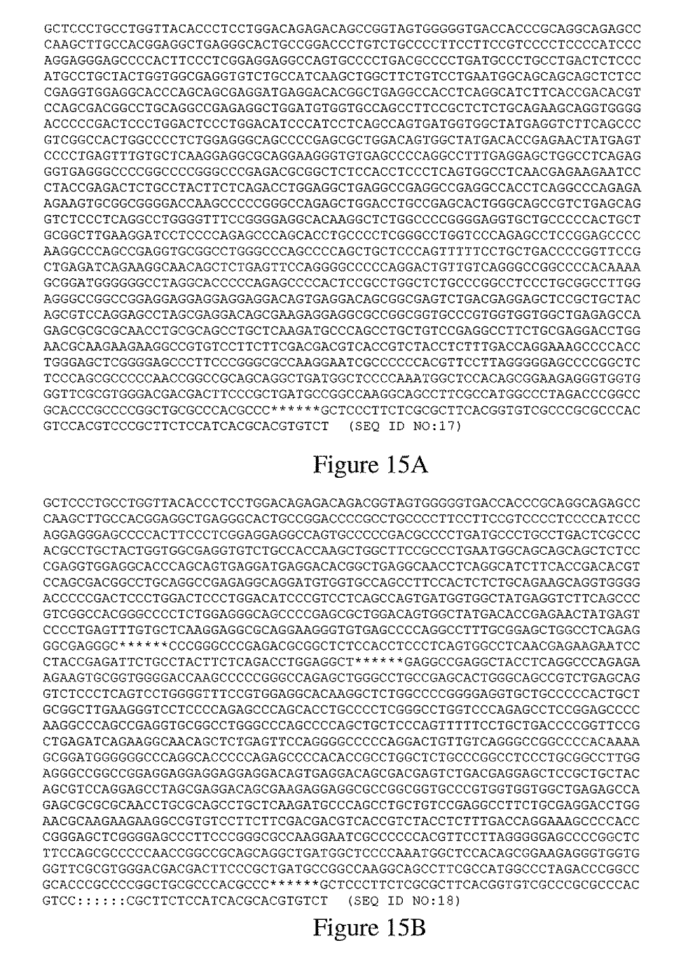

Chimpanzee DNA was sequenced as part of a high-throughput sequencing project on a MegaBACE 1000 sequencer (AP Biotech). DNA sequences were used as query sequences in a BLAST search of the GenBank database. Two random chimpanzee sequences, termed stch856 and stch610, returned results for two genes in the non-redundant database of GenBank: NM_{—}004920 (human apoptosis-associated tyrosine kinase, AATYK) and AB014541 (human KIAA641, identical nucleotide sequence to NM_{—}004920), shown in FIG. 14A, and also showed a high K_{A}/K_{S} ratio compared to these human sequences. Primers were designed for PCR and sequencing of AATYK. Sequence was obtained for the 3 prime end of this gene in chimp and gorilla. The 5 prime end of the gene was difficult to amplify, and no sequence was confirmed in human and gorilla. The human AATYK gene (SEQ ID NO:14) has a coding region of 3624 bp (nucleotides 413-4036 of SEQ ID NO:14), and codes for a protein of 1207 amino acids (SEQ ID NO:16). 1809 bp were sequenced in both chimp and gorilla. See FIGS. 15A and 15B. The partial sequences (SEQ ID NO:17 and SEQ ID NO:18) did not include the start or stop codons, although they were very close to the stop codon on the 3 prime end (21 codons away). These sequences correspond to nucleotides 2170-3976 or 2179-3988 of the corresponding human sequences taking into account the gaps described below.

There were also several pairs of amino acid insertions/deletions among chimp, human and gorilla in the coding region. The following sequences are in reading frame:

(SEQ ID NO: 19)

| | |
|---|---|
| Chimp | GGTGAGGGCCCCGGCCCCGGGCCC |

(SEQ ID NO: 20)

| | | | |
|---|---|---|---|
| Human | 2819 | GGTGAGGGC::::::CCCGGGCCC | 2836 |

(SEQ ID NO: 21)

| | |
|---|---|
| Gorilla | GGCGAGGGC::::::CCCGGGCCC |

(SEQ ID NO: 22)

| | |
|---|---|
| Chimp | CTGGAGGCTGAGGCCGAGGCCGAG |

(SEQ ID NO: 23)

| | | | |
|---|---|---|---|
| Human | 2912 | CTCGAGGCT::::::GAGGCCGAG | 2929 |

(SEQ ID NO: 24)

| | |
|---|---|
| Gorilla | CTGGAGGCT::::::GAGGCCGAG |

(SEQ ID NO: 25)

| | |
|---|---|
| Chimp | CCCACGCCC::::::GCTCCCTTC |

(SEQ ID NO: 26)

| | | | |
|---|---|---|---|
| Human | 3890 | CCCACGCCCACGCCCGCTCCCTTC | 3913 |

(SEQ ID NO: 27)

| | |
|---|---|
| Gorilla | CCCACGCCC::::::GCTCCCTTC |

(SEQ ID NO: 28)

| | |
|---|---|
| Chimp | CCCACGTCCACGTCCCGCTTCTCC |

(SEQ ID NO: 29)

| | | | |
|---|---|---|---|
| Human | 3938 | CCCACGTCC::::::CGCTTCTCC | 3955 |

(SEQ ID NO: 30)

| | |
|---|---|
| Gorilla | CCCACGTCC::::::CGCTTCTCC |

Each of these insertions/deletions affected two amino acids and did not change the reading frame of the sequence. Sliding window K_{A}/K_{S} for chimp to human, chimp to gorilla, and human to gorilla, excluding the insertion/deletion regions noted above, showed a high Ka/Ks ratio for some areas. See Table 9.

The highest Ka/Ks ratios are human to gorilla and chimp to gorilla, suggesting that both the human and chimp gene have undergone selection, and is consistent with the idea that the two species share some enhanced cognitive abilities relative to the other great apes (gorillas, for example). Such data bolsters the view that this gene may play a role with regard to enhanced cognitive functions. It should also be noted that in general, the human-containing pairwise comparisons are higher than the analogous chimp-containing comparisons.

TABLE 9
K_{A}/K_{S} ratios for various windows of AATYK on chimp, human, and gorilla

| | | | | | | | | | | bp of NM 004920 |
|---|---|---|---|---|---|---|---|---|---|---|
| AATYK | | K_{A} | K_{S} | K_{A}/K_{S} | K_{A} SE | K_{S} SE | size bp | bp of partial CDS | t | (pub human AATYK) |
| chimp | gorilla | 0.02287 | 0.03243 | 0.705211 | 0.00433 | 0.00832 | 1809 | 1-1809 | 1.019266 | 2180-3988 |
| chimp | human | 0.01538 | 0.01989 | 0.773253 | 0.00366 | 0.0062 | 1809 | 1-1809 | 0.626415 | 2180-3988 |
| human | gorilla | 0.02223 | 0.03204 | 0.69382 | 0.00429 | 0.00848 | 1809 | 1-1809 | 1.032263 | 2180-3988 |
| ch1 | hu1 | 0.03126 | 0.02009 | 1.555998 | 0.01834 | 0.02034 | 150 | 1-150 | 0.407851 | 2180-2329 |
| ch2 | hu2 | 0.03142 | 0.04043 | 0.777146 | 0.01844 | 0.02919 | 150 | 100-249 | 0.260958 | 2279-2428 |
| ch3 | hu3 | 0.02073 | 0.02036 | 1.018173 | 0.01481 | 0.02087 | 150 | 202-351 | 0.014458 | 2381-2530 |
| ch4 | hu4 | 0.02733 | 0.02833 | 0.964702 | 0.01753 | 0.02383 | 150 | 301-450 | 0.033803 | 2480-2629 |
| ch5 | hu5 | 0 | 0.05152 | 0 | 0 | 0.03802 | 150 | 400-549 | 1.355076 | 2579-2728 |
| ch6 | hu6 | 0.00836 | 0.03904 | 0.214139 | 0.00838 | 0.03964 | 150 | 502-651 | 0.75723 | 2681-2830 |
| ch7 | hu7 | 0.00888 | 0.05893 | 0.150687 | 0.0089 | 0.0439 | 150 | 601-750 | 1.11736 | 2780-2929 |
| ch8 | hu8 | 0.02223 | 0.03829 | 0.580569 | 0.01589 | 0.03886 | 150 | 700-849 | 0.382534 | 2879-3028 |
| ch9 | hu9 | 0.04264 | 0.03644 | 1.170143 | 0.02173 | 0.02628 | 150 | 799-948 | 0.181817 | 2978-3127 |
| ch10 | hu10 | 0.02186 | 0.01823 | 1.199122 | 0.01563 | 0.01851 | 150 | 901-1050 | 0.149837 | 3080-3229 |
| ch11 | hull | 0.01087 | 0 | #DIV/0! | 0.01093 | 0 | 150 | 1000-1149 | 0.994511 | 3179-3328 |
| ch12 | hu12 | 0.01093 | 0 | #DIV/0! | 0.01099 | 0 | 150 | 1099-1248 | 0.99454 | 3278-3427 |
| ch13 | hu13 | 0.01031 | 0 | #DIV/0! | 0.01036 | 0 | 150 | 1201-1350 | 0.995174 | 3380-3529 |
| ch14 | hu14 | 0.01053 | 0 | #DIV/0! | 0.01058 | 0 | 150 | 1300-1449 | 0.995274 | 3479-3628 |
| ch15 | hu15 | 0.01835 | 0.02006 | 0.914756 | 0.01315 | 0.02057 | 150 | 1399-1548 | 0.070042 | 3578-3727 |
| ch16 | hu16 | 0 | 0.02027 | 0 | 0 | 0.02062 | 150 | 1501-1650 | 0.983026 | 3680-3829 |
| ch17 | hu17 | 0.00666 | 0 | #DIV/0! | 0.00667 | 0 | 210 | 1600-1809 | 0.998501 | 3779-3988 |
| chA | huA | 0.02366 | 0.02618 | 0.903743 | 0.00875 | 0.01251 | 501 | 1-501 | 0.165069 | 2180-2680 |
| chB | huB | 0.01159 | 0.03863 | 0.300026 | 0.00585 | 0.01811 | 501 | 400-900 | 1.420809 | 2579-3079 |
| chC | huC | 0.02212 | 0.0108 | 2.048148 | 0.00846 | 0.00768 | 501 | 799-1299 | 0.990721 | 2978-3478 |
| chD | huD | 0.00851 | 0.00734 | 1.159401 | 0.00458 | 0.00602 | 609 | 1201-1809 | 0.154676 | 3380-3988 |
| chA | gorA | 0.02082 | 0.04868 | 0.427691 | 0.00795 | 0.0191 | 501 | 1-501 | 1.346644 | 2180-2680 |
| chB | gorB | 0.01416 | 0.04039 | 0.350582 | 0.00639 | 0.0172 | 501 | 400-900 | 1.429535 | 2579-3079 |
| chC | gorC | 0.01737 | 0.00538 | 3.228625 | 0.00717 | 0.00542 | 501 | 799-1299 | 1.333991 | 2978-3478 |
| chD | gorD | 0.00644 | 0.00244 | 2.639344 | 0.00408 | 0.00346 | 609 | 1201-1809 | 0.747722 | 3380-3988 |
| huA | gorA | 0.02246 | 0.02759 | 0.814063 | 0.00829 | 0.01523 | 501 | 1-501 | 0.295847 | 2180-2680 |
| huB | gorB | 0.01418 | 0.06809 | 0.208254 | 0.0064 | 0.02388 | 501 | 400-900 | 2.180583 | 2579-3079 |
| huC | gorC | 0.01993 | 0.00541 | 3.683919 | 0.00762 | 0.00544 | 501 | 799-1299 | 1.550854 | 2978-3478 |
| huD | gorD | 0.00723 | 0.00488 | 1.481557 | 0.0042 | 0.0049 | 609 | 1201-1809 | 0.364133 | 3380-3988 |

### Example 14

### Positively Selected Human BRCA1 Gene

Comparative evolutionary analysis of the BRCA1 genes of several primate species has revealed that the human BRCA1 gene has been subjected to positive selection. Initially, 1141 codons of exon 11 of the human and chimpanzee BRCA1 genes (Hacia et al. (1998) Nature Genetics 18:155-158) were compared and a strikingly high K_{A}/K_{S} ratio, 3.6, was found when calculated by the method of Li (Li (1993) J. Mol. Evol. 36:96-99; Li et al. (1985) Mol. Biol. Evol. 2:150-174). In fact, statistically significant elevated ratios were obtained for this comparison regardless of the particular algorithm used (see Table 5A). Few genes (or portions of genes) have been documented to display ratios of this magnitude (Messier et al. (1997) Nature 385:151-154; Endo et al. (1996) Mol. Biol. Evol. 13:685-690; and Sharp (1997) Nature 385:111-112). We thus chose to sequence the complete protein-coding region (5589 bp) of the chimpanzee BRCA1 gene, in order to compare it to the full-length protein-coding sequence of the human gene. In many cases, even when positive selection can be shown to have operated on limited regions of a particular gene, K_{A}/K_{S} analysis of the full-length protein-coding sequence fails to reveal evidence of positive selection (Messier et al. (1997), supra). This is presumably because the signal of positive selection can be masked by noise when only small regions of a gene have been positively selected, unless selective pressures are especially strong. However, comparison of the full-length human and chimpanzee BRCA1 sequences still yielded K_{A}/K_{S} ratios in excess of one, by all algorithms we employed (Table 5A). This suggests that the selective pressure on BRCA1 was intense. A sliding-window K_{A}/K_{S} analysis was also performed, in which intervals of varying lengths (from 150 to 600 bp) were examined, in order to determine the pattern of selection within the human BRCA1 gene. This analysis suggests that positive selection seems to have been concentrated in exon 11.

TABLE 5A
Human-Chimpanzee K_{A}/K_{S} Comparisons

| Method | K_{A}/K_{S} (exon 11) | K_{A}/K_{S} (full-length) |
|---|---|---|
| Li (1993) J. Mol. Evol. 36: 96; | 3.6*** | 2.3* |
| Li et al. (1985) Mol Biol Evol. | | |
| 2: 150 | | |
| Ina Y. (1995) J. Mol. Evol. | 3.3** | 2.1* |
| 40: 190 | | |
| Kumar et al., MEGA: Mol. | 2.2* | 1.2 |
| Evol. Gen. Anal. (PA St. Univ, | | |
| 1993) | | |

TABLE 5B
K_{A}/K_{S} for Exon 11 of BRCA1 from Additional Primates

| Comparison | | K_{A} | K_{S} | K_{A}/K_{S} |
|---|---|---|---|---|
| Human | Chimpanzee | 0.010 | 0.003 | 3.6* |
| | Gorilla | 0.009 | 0.009 | 1.1 |
| | Orangutan | 0.018 | 0.020 | 0.9 |
| Chimpanzee | Gorilla | 0.006 | 0.007 | 0.8 |
| | Orangutan | 0.014 | 0.019 | 0.7 |
| Gorilla | Orangutan | 0.014 | 0.025 | 0.6 |

The Table 5B ratios were calculated according to Li (1993) J. Mol. Evol. 36:96; Li et al. (1985) Mol. Biol. Evol. 2:150. For all comparisons, statistical significance was calculated by t-tests, as suggested in Zhang et al. (1998) Proc. Natl. Acad. Sci. USA 95:3708. Statistically significant comparisons are indicated by one or more asterisks, with P values as follows: *, P<0.05, **, P<0.01, ***, P<0.005. Exon sequences are from Hacia et al. (1998) Nature Genetics 18:155. GenBank accession numbers: human, NM_{—}000058.1, chimpanzee, AF019075, gorilla, AF019076, orangutan, AF019077, rhesus, AF019078.

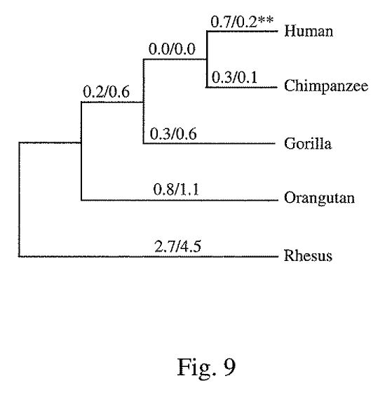

The elevated K_{A}/K_{S} ratios revealed by pairwise comparisons of the human and chimpanzee BRCA1 sequences demonstrate the action of positive selection, but such comparisons alone do not reveal which of the two genes compared, the human or the chimpanzee, has been positively selected. However, if the primate BRCA1 sequences are considered in a proper phylogenetic framework, only those pairwise comparisons which include the human gene show ratios greater than one, indicating that only the human gene has been positively selected (Table 5B). To confirm that positive selection operated on exon 11 of BRCA1 exclusively within the human lineage, the statistical test of positive selection proposed by Zhang et al. (1998) Proc. Natl. Acad. Sci. USA 95:3708-3713, was used. This test is especially appropriate when the number of nucleotides is large, as in the present case (3423 bp). This procedure first determines nonsynonymous nucleotide substitutions per nonsynonymous site (b_{N}) and synonymous substitutions per synonymous site (b_{S}) for each individual branch of a phylogenetic tree (Zhang et al. (1998), supra). Positive selection is supported only on those branches for which b_{N}−b_{S} can be shown to be statistically significant (Zhang et al. (1998), supra). For BRCA1, this is true for only one branch of the primate tree shown in FIG. 9: the branch which leads from the human/chimpanzee common ancestor to modern humans, where b_{N}/b_{S}=3.6. Thus, we believe that in the case of the BRCA1 gene, positive selection operated directly and exclusively on the human lineage.

While it is formally possible that elevated K_{A}/K_{S} ratios might reflect some locus or chromosomal-specific anomaly (such as suppression of K_{S} due, for example, to isochoric differences in GC content), rather than the effects of positive selection, this is unlikely in the present case, for several reasons. First, the estimated K_{S} values for the hominoid BRCA1 genes, including human, were compared to those previously estimated for other well-studied hominoid loci, including lysozyme (Messier et al. (1997), supra) and ECP (Zhang et al (1998), supra). There is no evidence for a statistically significant difference in these values. This argues against some unusual suppression of K_{S} in human BRCA1. Second, examination of GC content (Sueoka, N. in Evolving Genes and Proteins (eds. Bryson, V. & Vogel, H.J.) 479-496 (Academic Press, NY, 1964)) and codon usage patterns (Sharp et al. (1988) Nucl. Acids Res. 16:8207-8211) of the primate BRCA1 genes shows no significant differences from average mammalian values.

This demonstration of strong positive selection on the human BRCA1 gene constitutes the first molecular support for a theory long advanced by anthropologists. Human infants require, and receive, prolonged periods of post-birth care—longer than in any of our close primate relatives. Short, R. V. (1976) Proc. R. Soc. Lond. B 195:3-24, first postulated that human females can only furnish such extended care to human infants in the context of a long term pair bond with a male partner who provides assistance. The maintenance of long term pair bonds was strengthened by development of exaggerated (as compared to our close primate relatives) human secondary sex characteristics including enlarged female breasts (Short (1976), supra). Thus, strong selective pressures resulted in development of enlarged human breasts which develop prior to first pregnancy and lactation, contrary to the pattern seen in our hominoid relatives (Dixson, A. F. in Primate Sexuality: Comparative Studies of the Prosimians, Monkeys, Apes and Human Beings. 214 (Oxford Univ. Press, Oxford, 1998)).

Evidence suggests that in addition to its function as a tumor suppressor (Xu et al. (1999) Mol. Cell. 3(3):389-395; Shen et al. (1998) Oncogene 17(24):3115-3124; Dennis, C. (1999) Nature Genetics 22:10; and Xu et al. (1999) Nature Genetics 22:37-43), the BRCA1 protein plays an important role in normal development of breast tissue (Dennis, C. (1999), supra; Xu et al (1999) Nature Genetics 22:37-43; and Thompson et al (1999) Nature Genetics 9:444-450), particularly attainment of typical mammary gland and duct size (Dennis, C. (1999), supra; and Xu et al. (1999) Nature Genetics 22:37-43). These facts suggest that positive selection on this gene in humans promoted expansion of the female human breast, and ultimately, helped promote long term care of dependent human infants. This long term dependency of human infants was essential for the development and transmission of complex human culture. Because positive selection seems to have been concentrated upon exon 11 of BRCA1, the prediction follows that the region of the BRCA1 protein encoded by exon 11 specifically plays a role in normal breast development. The data provided here suggests that strong selective pressures during human evolution led to amino acid replacements in BRCA1 that promoted a unique pattern of breast development in human females, which facilitated the evolution of some human behaviors.

### Example 15

### Characterization of BRCA1 Polynucleotide and Polypeptide

Having identified evolutionarily significant nucleotide changes in the BRCA1 gene and corresponding amino acid changes in the BRCA1 protein, the next step is to test these molecules in a suitable model system to analyze the functional effect of the nucleotide and amino acid changes on the model. For example, the human BRCA1 polynucleotide can be transfected into a cultured host cell such as adipocytes to determine its effect on cell growth or replication.

### Example 16

### Identification of Positively-Selected CD59

Comparative evolutionary analysis of the CD59 genes of several primate species has revealed that the chimpanzee CD59 gene has been subjected to positive selection. CD59 protein is also known as protectin, IF-5Ag, H19, HRF20, MACIF, MIRL, and P-18. CD59 is expressed on all peripheral blood leukocytes and erythrocytes (Meri et al. (1996) Biochem. J. 316:923-935). Its function is to restrict lysis of human cells by complement (Meri et al. (1996), supra). More specifically, CD59 acts as one of the inhibitors of membrane attack complexes (MACs). MACs are complexes of 20 some complement proteins that make hole-like lesions in cell membranes (Meri et al (1996), supra). These MACs, in the absence of proper restrictive elements (i.e., CD59 and a few other proteins) would destroy host cells as well as invading pathogens. Essentially then, CD59 protects the cells of the body from the complement arm of its own defense systems (Meri et al (1996), supra). The chimpanzee homolog of this protein was examined because the human homolog has been implicated in progression to AIDS in infected individuals. It has been shown that CD59 is one of the host cell derived proteins that is selectively taken up by HIV virions (Frank et al. (1996) AIDS 10: 1611-1620). Additionally, it has been shown (Saifuddin et al. (1995) J. Exp. Med. 182:501-509) that HIV virions which have incorporated host cell CD59 are protected from the action of complement. Thus it appears that in humans, HIV uses CD59 to protect itself from attack by the victim=s immune system, and thus to further the course of infection.

To obtain primate CD59 cDNA sequences, total RNA was prepared (using either the RNeasy® kit (Qiagen), or the RNAse-free Rapid Total RNA kit (5 Prime -3 Prime, Inc.)) from primate tissues (whole fresh blood from chimpanzees, gorillas, and orangutans). mRNA was isolated from total RNA using the Mini-Oligo(dT) Cellulose Spin Columns (5 Prime -3 Prime, Inc.). cDNA was synthesized from mRNA with oligo dT and/or random priming using the SuperScript Preamplification System for First Strand cDNA Synthesis (Gibco BRL). The protein-coding region of the primate CD59 gene was amplified from cDNA using primers (concentration=100 nmole/μl) designed from the published human sequence. PCR conditions for CD59 amplification were 94EC initial pre-melt (4 min), followed by 35 cycles of 94EC (15 sec), 58EC (1 min 15 sec), 72EC (1 min 15 sec), and a final 72EC extension for 10 minutes. PCR was accomplished on a Perkin-Elmer GeneAmp7 PCR System 9700 thermocycler, using Ready-to-Go PCR beads (Amersham Pharmacia Biotech) in a 50 μl total reaction volume. Appropriately-sized products were purified from agarose gels using the QiaQuick Gel Extraction kit (Qiagen). Both strands of the amplification products were sequenced directly using the Big Dye Cycle Sequencing Kit and analyzed on a 373A DNA sequencer (ABI BioSystems).

As shown in Table 6, all comparisons to the chimpanzee CD59 sequence display K_{A}/K_{S} ratios greater than one, demonstrating that it is the chimpanzee CD59 gene that has been positively-selected.

TABLE 6
K_{A}/K_{S} Ratios for Selected Primate CD59 cDNA Sequences

| Genes Compared | K_{A}/K_{S} Ratios |
|---|---|
| Chimpanzee to Human | 1.8 |
| Chimpanzee to Gorilla | 1.5 |
| Chimpanzee to Orangutan | 2.3 |
| Chimpanzee to Green Monkey | 3.0 |

### Example 17

### Characterization of CD59 Positively-Selected Sequences

Proceeding on the hypothesis that strong selection pressure has resulted in adaptive changes in the chimpanzee CD59 molecule such that disease progression is retarded because the virus is unable to usurp CD59=s protective role for itself, it then follows that comparisons of the CD59 gene of other closely-related non-human primates to the human gene should display K_{A}/K_{S} ratios less than one for those species that have not been confronted by the HIV-1 virus over evolutionary periods. Conversely, all comparisons to the chimpanzee gene should display K_{A}/K_{S} ratios greater than one. These two tests, taken together, will definitively establish whether the chimpanzee or human gene was positively selected. Although the gorilla (Gorilla gorilla) is the closest relative to humans and chimpanzees, its postulated historical range in Africa suggests that gorillas could have been at some time exposed to the HIV-1 virus. We thus examined the CD59 gene from both the gorilla and the orangutan (Pongo pygmaeus). The latter species, confined to Southeast Asia, is unlikely to have been exposed to HIV over an evolutionary time frame. The nucleotide sequences of the human and orangutan genes were determined by direct sequencing of cDNAs prepared from RNA previously isolated from whole fresh blood taken from these two species.

The next step is to determine how chimpanzee CD59 contributes to chimpanzee resistance to progression to full-blown AIDS using assays of HIV replication in cell culture. Human white blood cell lines, transfected with, and expressing, the chimpanzee CD59 protein, should display reduced rates of viral replication (using standard assays familiar to practitioners of the art) as compared to control lines of untransfected human cells. In contrast, chimpanzee white blood cell lines expressing human CD59 should display increased viral loads as compared to control, untransfected chimpanzee cell lines.

### Example 18

### Molecular Modeling of CD59

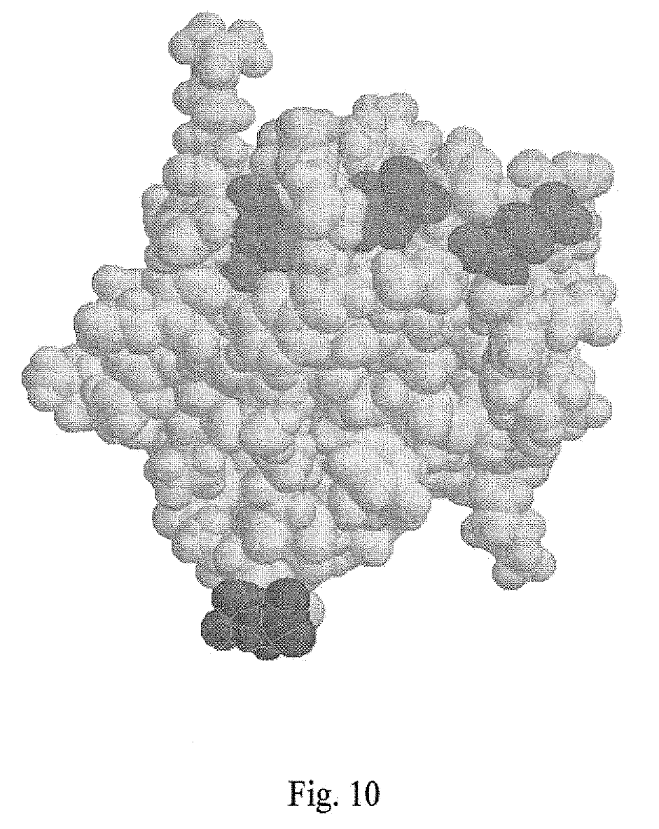

Modeling of the inferred chimpanzee protein sequence of CD59 upon the known three-dimensional structure of human (Meri et al. 1996 Biochem J. 316:923-935) has provided additional evidence for the role of this protein in explaining chimpanzee resistance to AIDS progression. It has been shown that in human CD59, residue Asn 77 is the link for the GPI anchor (Meri et al. (1996) Biochem J. 316:923-935), which is essential for function of the protein. The GPI anchor is responsible for anchoring the protein to the cell membrane (Meri et al. (1996), supra). Our sequencing of the chimpanzee CD59 gene reveals that the inferred protein structure of chimpanzee CD59 contains a duplication of the section of the protein that contains the GPI link, i.e., NEQLENGG (see Table 7 and FIG. 10).

TABLE 7
Comparison of Human and Chimpanzee CD59 Amino
Acid Sequence

| | |
|---|---|
| Human | SLQCYNCPNP TADCKTAVNC SSDFDACLIT KAGLQVYNKC |
| Chimpanzee | SLQCYNCPNP TADCKTAVNC SSDFDACLIT KAGLQVYNKC |
| Human | WKFEHCNFND VTTRLRENEL TYYCCKKDLC NFNEQLENGG |
| Chimpanzee | WKLEHCNFKD LTTRLRENEL TYYCCKKDLC NFNEQLENGG |
| Human | -----------------TSLS EKTVLLLVTP FLAAAAWSLHP |
| Chimpanzee | NEQLENGGNE QLENGGTSLS EKTVLLRVTP FLAAAAWSLHP |
| Human | (SEQ ID NO: 12) |
| Chimpanzee | (SEQ ID NO: 13) |

Italics/underline indicates variation in amino acids.

This suggests that while the basic function of CD59 is most likely conserved between chimpanzee and human, some changes have probably occurred in the orientation of the protein with respect to the cell membrane. This may render the chimpanzee protein unusable to the HIV virion when it is incorporated by the virion. Alternatively, the chimpanzee protein may not be subject to incorporation by the HIV virion, in contrast to the human CD59. Either of these (testable) alternatives would likely mean that in the chimpanzee, HIV virions are subject to attack by MAC complexes. This would thus reduce amounts of virus available to replicate, and thus contribute to chimpanzee resistance to progression to full-blown AIDS. Once these alternatives have been tested to determine which is correct, then the information can be used to design a therapeutic intervention for infected humans that mimics the chimpanzee resistance to progression to full-blown AIDS.

### Example 19

### Identification of Positively-Selected DC-SIGN

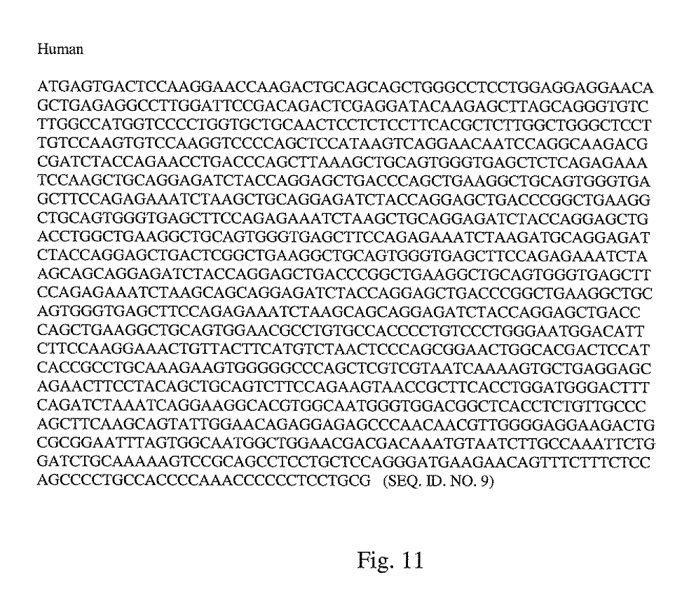

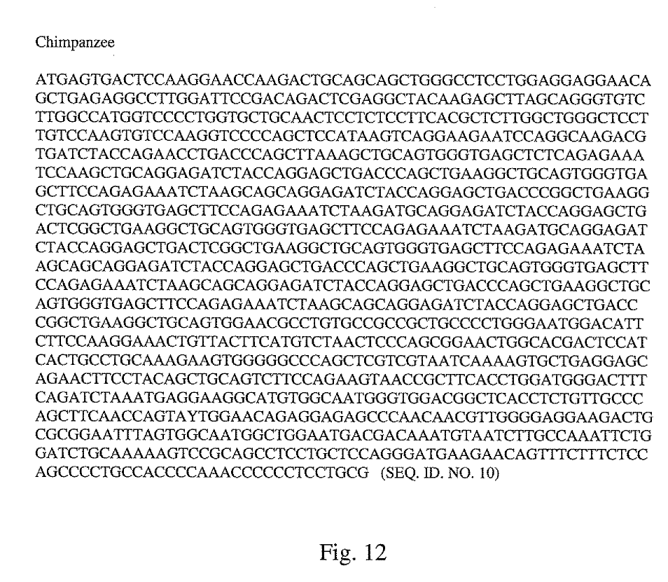

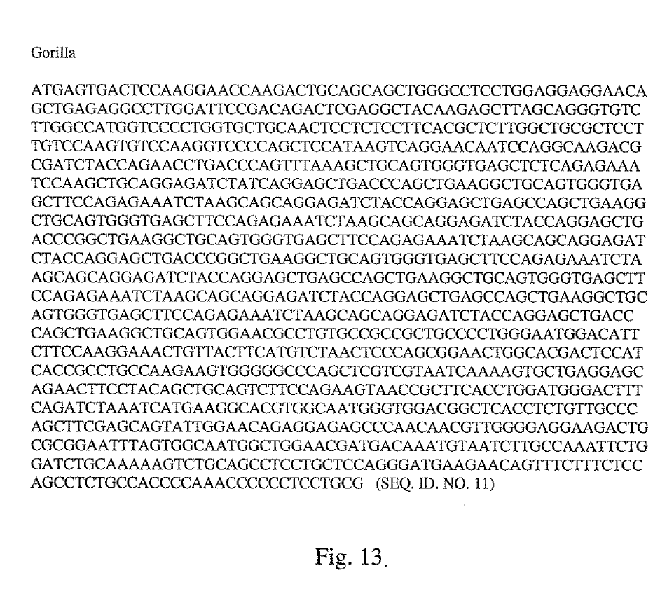

Comparative evolutionary analyses of DC-SIGN genes of human, chimpanzee and gorilla have revealed that the chimpanzee DC-SIGN gene has been subjected to positive selection. FIGS. 11-13 (SEQ. ID. NOS. 6-8) show the nucleotide sequences of human, chimpanzee and gorilla DC-SIGN genes, respectively. Table 8 provides the K_{A}/K_{S} values calculated by pairwise comparison of the human, chimpanzee and gorilla DC-SIGN genes. Note that only those comparisons with chimpanzee show K_{A}/K_{S} values greater than one, indicating that the chimpanzee gene has been positively selected.

TABLE 8
K_{A}/K_{S} Ratios for Selected Primate DC-SIGN cDNA Sequences

| Genes Compared | K_{A}/K_{S} Ratios |
|---|---|
| Chimpanzee to Human | 1.3 |
| Human to Gorilla | 0.87 |
| Chimpanzee to Gorilla | 1.3 |

As discussed herein, DC-SIGN is expressed on dendritic cells and is known to provide a mechanism for transport of HIV-1 virus to the lymph nodes. HIV-1 binds to the extracellular portion of DC-SIGN and infects the undifferentiated T cells in the lymph nodes via their CD4 proteins. This expansion in infection ultimately leads to compromise of the immune system and subsequently to full-blown AIDS. Interestingly, DC-SIGNS's major ligand appears to be ICAM-3. As described herein, chimpanzee ICAM-3 shows the highest K_{A}/K_{S} ratio of any known AIDS-related protein. It is not yet clear whether positive selection on chimpanzee ICAM-3 was a result of compensatory changes that allow ICAM-3 to retain its ability to bind to DC-SIGN.

### Example 20

### Detection of Positive Selection upon Chimpanzee p44

As is often true, whole protein comparisons for human and chimpanzee p44 display K_{A}/K_{S} ratios less than one. This is because the accumulated “noise” of silent substitutions in the full-length CDS can obscure the signal of positive selection if it has occurred in a small section of the protein. However, examination of exon 2 of the chimpanzee and human homologs reveals that this portion of the gene (and the polypeptide it codes for) has been positively selected. The K_{A}/K_{S} ratio for exon 2 is 1.5 (P<0.05). Use of this invention allowed identification of the specific region of the protein that has been positively selected.

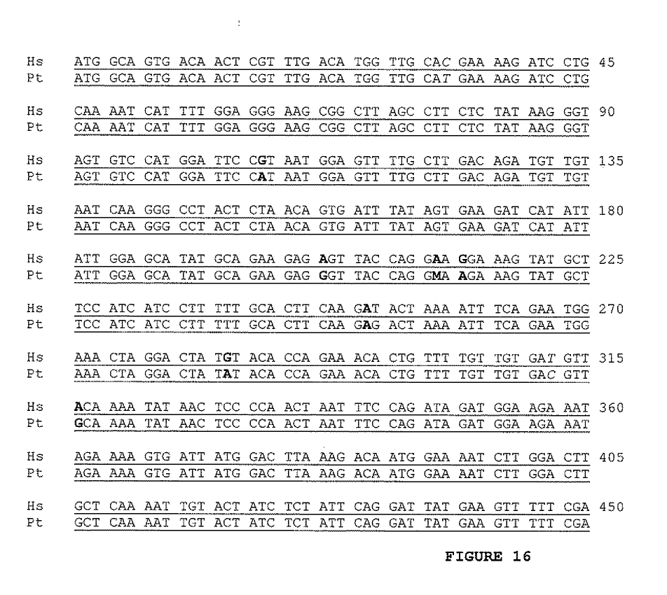

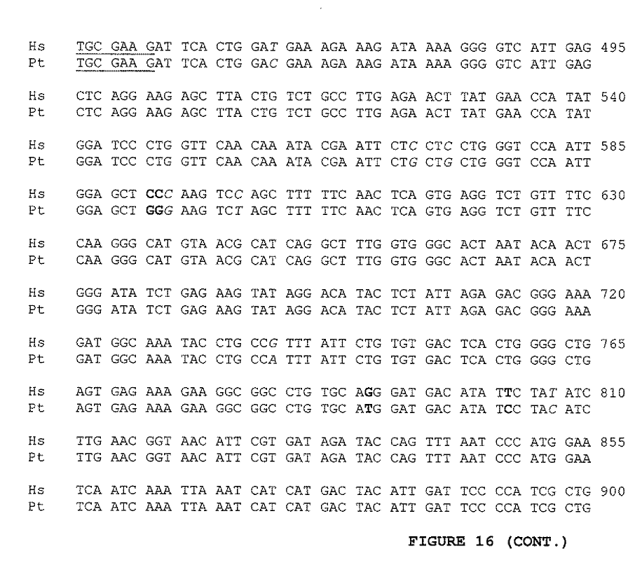

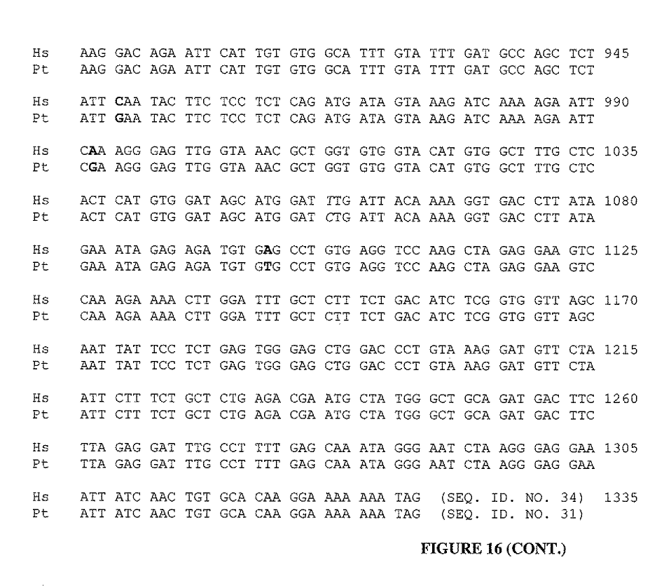

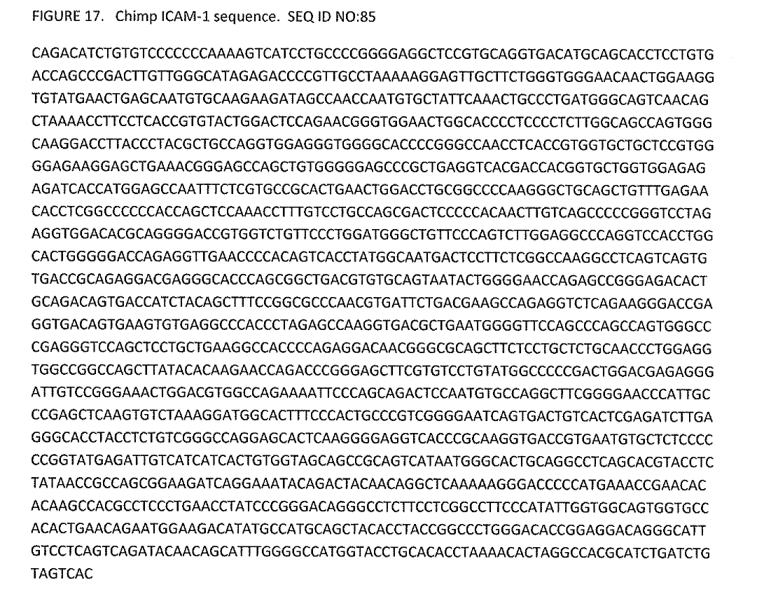

Two alleles of p44 were detected in chimpanzees that differ by a single synonymous substitution (see FIG. 16). For human to chimpanzee, the whole protein K_{A}/K_{S} ratio for allele A is 0.42, while the ratio for allele B is 0.45.

In FIG. 16, the CDS of human (Acc. NM_{—}006417) and chimpanzee (Acc. D90034) p44 gene are aligned, with the positively selected exon 2 underlined (note that exon 2 begins at the start of the CDS, as exon 1 is non-coding.). Human is labeled Hs (Homo sapiens), chimpanzee is labeled Pt (Pan troglodytes). Nonsynonymous differences between the two sequences are in bold, synonymous differences are in italics. Chimpanzee has a single heterozygous base (position 212), shown as “M”, using the IUPAC code to signify either adenine (“A”) or cytosine (“C”). Note that one of these (“C”) represents a nonsynonymous difference from human, while “A” is identical to the same position in the human homolog. Thus these two chimpanzee alleles differ slightly in their K_{A}/K_{S} ratios relative to human p44.

### Example 21

### Methods for Screening Agents that May be Useful in Treatment of HCV in Humans

Candidate agents can be screened in vitro for interaction with purified p44, especially exon 2. Candidate agents can be designed to interact with human p44 exon 2 so that human p44 can mimic the structure and/or function of chimpanzee p44. Human and chimpanzee p44 are known and can be synthesized using methods known in the art.

Molecular modeling of small molecules to dock with their targets, computer assisted new lead design, and computer assisted drug discovery are well known in the art and are described, e.g., in Cohen, N.C. (ed.) Guidebook on Molecular Modeling in Drug Design, Academic Press (1996). Additionally, there are numerous commercially available molecular modeling software packages.

Affinity chromatography can be used to partition candidate agents that bind in vitro to human p44 (especially exon 2) from those that do not. It may also be useful to partition candidate agents that no only bind to human p44 exon 2, but also do not bind to chimpanzee p44 exon 2, so as to eliminate those agents that are not specific to the human p44 exon 2.

Optionally, x-ray crystallography structures of p44-agent complexes can be compared to x-ray structures of human p44 and chimpanzee p44 to determine if the human p44-agent complexes more closely resemble x-ray structures of chimpanzee p44 structures.

Further, candidate agents can be screened for favorable interactions with p44 during HCV infection of hepatocytes in vitro. Fournier et al. (1998) J. Gen. Virol. 79:2367 report that adult normal human hepatocytes in primary culture can be successfully infected with HCV and used as an in vitro HCV model (see also Rumin et al. (1999) J. Gen. Virology 80:3007). Favre et al. (2001) CR Acad. Sci. III 324(12):1141-8, report that a robust in vitro infection of hepatocytes with HCV is facilitated by removal of cell-bound lipoproteins prior to addition of viral inocula from human sera. Further, Kitamura et al. (1994) Eur. J. Biochem. 224:877-83, report that IFNα/β induces human p44 gene in hepatocytes in vitro. The p44 protein is produced in vivo in infected human livers (Patzwahl, R. et al. (2000) J. Virology 75(3): 1332). While it is presently not clear if p44 is produced by human hepatocytes in vitro during HCV infection, if it is not, IFNα/β could be added to induce p44. This in vitro system could serve as a suitable model for screening candidate agents for their capacity to favorably interact with human p44 in HCV infected hepatocytes.

An assay for favorable interaction of candidate agents with p44 in in vitro cultured cells could be the enhancement of p44 assembly into microtubules in the cultured hepatocytes. Assembled chimpanzee p44 microtubular aggregates associated with NANB hepatitis infection in chimpanzees have been detected by antibodies described in Takahashi, K. et al. (1990) J. Gen. Virology 71(Pt9):2005-11. These antibodies may be useful in detecting human p44 microtubular aggregates. Alternatively, antibodies to human p44 can be made using methods known in the art.

A direct link between enhanced p44 microtubular assembly and increased resistance to HCV infection in chimpanzees or humans is not known at this time. However, the literature does indicate that increased p44 microtubular assembly is associated with HCV infection in chimpanzees, and chimpanzees are able to resist HCV infection. Specifically, Patzwahl, R. et al. (2000) J. Virology 75:1332-38, reports that p44 is a “component of the double-walled membranous tubules which appear as a distinctive alteration in the cytoplasm of hepatocytes after intravenous administration of human non-A, non-B (NANB) hepatitis inocula in chimpanzees.” Likewise, Takahashi, K. et al. (1990) J. Gen. Virology 71(Pt9):2005-11, report that p44 is expressed in NANB hepatitis infected chimpanzees and is a host (and not a viral) protein. Additionally, Patzwahl, R. et al. (2000), supra, report that p44 expression is increased in HCV infected human livers; it is not clear whether the human p44 assembles into microtubules. Finally, Kitamura, A. et al. (1994) Eur. J. Biochem. 224:877 suggest at page 882 that “p44 may function as a mediator of anti-viral activity of interferons against hepatitis C . infection, through association with the microtubule aggregates.”

A suitable control could be in vitro cultured chimpanzee hepatocytes that are infected with HCV, and which presumably would express p44 that assembles into microtubules and resist the HCV infection.

The foregoing in vitro model could serve to identify those candidate agents that interact with human p44 to produce a function (microtubule assembly or HCV resistance) that is characteristic of chimpanzee p44 during HCV infection. Candidate agents can also be screened in in vivo animal models for inhibition of HCV. Several in vivo human HCV models have been described in the literature. Mercer, D. et al. (2001) Nat. Med. 7(8):927-33, report that a suitable small animal model for human HCV is a SCID mouse carrying a plasminogen activator transgene (Alb-uPA) with transplanted normal human hepatocytes. The mice have chimeric human livers, and when HCV is administered via inoculation with infected human serum, serum viral titres increase. HCV viral proteins were localized to the human hepatocyte nodules.

Galun, E. et al. (1995) describe a chimeric mouse model developed from BNX (beige/nude/X-linked immunodeficient) mice preconditioned by total body irradiation and reconstituted with SCID mouse bone marrow cells, into which were implanted HCV-infected liver fragments from human patients, or normal liver incubated with HCV serum.

LaBonte, P. et al. (2002) J. Med. Virol. 66(3):312-9, describe a mouse model developed by orthotopic implantation of human hepatocellular carcinoma cells (HCC) into athymic nude mice. The human tumors produce HCV RNA.

Any of the foregoing mouse models could be treated with IFN-α/β to induce p44 production (if necessary), and candidate agents could be added to detect any inhibition in HCV infection by, e.g., reduction in serum viral titer.

As a control, chimpanzee liver hepatocytes can be implanted into SCID or another suitable mouse to create a chimeric liver, and infected with HCV. Presumably, the chimp livers in the control mouse model would express p44 and be more resistant to HCV infection.

The experimental mice with the human hepatocytes are administered candidate agents and the course of the HCV infection (e.g., viral titres) is then monitored in the control and experimental models. Those agents that improve resistance in the experimental mice to the point where the human p44 function approaches (or perhaps exceeds) the chimpanzee p44 function in the control mouse model, are agents that may be suitable for human clinical trials.

### Example 22

### Structure/Function Implications of Changes in Chimpanzee ICAMs

Using published crystal structures, we examined the locations of the unique chimpanzee amino acid replacements in ICAM 1, with respect to amino acids that are critical for binding and dimerization (Casasnovas et al. (1998) Proc. Natl. Acad. Sci, USA 95:4134-4139; Bella et al (1998) Proc. Natl. Acad. Sci. USA 95:4140-4145).

One of the amino acid replacements we found to be unique to the chimpanzee lineage is Leu-18 (replaced by the more hydrophilic Glu-18), one of the leucines in a leucine cluster that creates a hydrophobic dimerization surface critical for human ICAM 1 dimerization (Jun et al. (2001)J. Biol. Chem. 276:29019-29027) (hydrophobicity score of 3.8 Leu replace by −3.5 Glu). The distortion of the hydrophobic surface in chimpanzee ICAM 1 suggests that selective pressure may have been directed towards mediating ICAM 1 dimerization in the chimpanzee.

In contrast, we found that all ICAM 1 residues thought to be involved in human LFA-1 binding (Diamond et al. (1991) Cell 65:961-971; Fisher et al. (1997) Mol. Biol. Cell 8:501-515; Edwards et al. (1998) J. Biol. Chem. 273:28937-28944; Shimaoka et al. (2003) Cell 112:99-111) are identical in chimpanzee and human ICAM 1. Indeed, these critical residues are highly conserved in all of the primate ICAMs we examined. Moreover, we found that the residues in the LFA-1 protein critical for binding to ICAM 1 (Shimaoka et al., 2003; Huth et al. (2000) Proc. Natl. Acad. Sci. USA 97:5231-5236), as well as for binding to ICAM 2 and ICAM 3 are also identical between chimpanzee and human. Our pairwise Ka/Ks comparisons of the chimpanzee and human LFA-1 genes also suggest conservation. (The LFA-1 protein contains two subunits, designated alpha and beta: Human LFA-1 alpha subunit to the chimpanzee LFA-1 alpha subunit: Ka/Ks=0.30; Human LFA-1 beta subunit to the chimpanzee LFA-1 beta subunit: Ka/Ks=0.053.). Thus, it is likely that the ICAM 1/LFA-1 binding interaction is fundamentally the same between humans and chimpanzees, except for the influence of the state of ICAM 1 dimerization, which, as described above, does appear to have been modulated in the chimpanzee as a result of adaptive evolution.

One of the unique chimpanzee ICAM 1 replacements we identified, Lys-29 to Asp-29, is immediately adjacent to a cluster of ICAM 1/LFA-1 binding residues, particularly Asn-66, which forms part of the contact surface for ICAM 11 LFA-1 binding. The amide side chain of Asn-66 is known to interact with Glu-241 of LFA-1, an interaction that has been shown to be absolutely critical for ICAM 1/LFA-1 binding. The interaction of Asn-66 with Glu-241 may be influenced by the replacement of the basic Lys-29 (humans) with the acidic Asp-29 (chimpanzee).

Lys-29 is reported to be a binding amino acid for the major group of human rhinoviruses, which use human ICAM 1 as a receptor (Register et al. (1991) J. Virol. 65:6589-6596). We considered the possibility that the selective force acting upon chimpanzee ICAM 1 was exposure to the rhinoviruses. Residue 49 is the only other rhinovirus-binding site that differs between chimpanzee and human; in this case, the chimpanzee sequence retains the ancestral Trp, while human shows a derived Arg, i.e., the human ICAM 1 sequence has changed, while the chimpanzee sequence has been conserved. Thus, this site provides evidence that exposure to rhinoviruses was not a selective force on chimpanzee ICAM 1.

As noted above, ICAM 1 also binds Mac-1. As for LFA-1, it appears unlikely that the binding interaction of ICAM 1 and Mac-1 has been the target of positive selection between chimpanzees and humans, for three reasons. First, our pairwise comparisons of the chimpanzee and human Mac-1 genes suggest conservation. (Like LFA-1, Mac-1 contains an alpha and a beta subunit. Human Mac-1 alpha subunit to the chimpanzee alpha subunit: Ka/Ks=0.30. Human Mac-1 beta subunit to the chimpanzee Mac-1 beta subunit, Ka/Ks=0.42). Second, domain 3 of ICAM 1 has long been known to be critical for Mac-1 binding (Diamond et al., 1991). As noted above, unlike domains 1 and 2, this domain is well conserved between humans and chimpanzee ICAM 1. Third, we found that ICAM 1 residues shown to be critical (Diamond et al., 1991) for Mac-1 binding (Asp-229, Asn-240, Glu-254, Asn-269) are identical between human and chimpanzee ICAM 1; indeed these are almost completely identical in all primate ICAM 1 sequences examined.

While de Groot et al. (2002 Proc. Natl. Acad. Sci. U.S.A. 99:11748-11753) suggest that chimpanzee resistance to progression to AIDS may result from the limited set of MHC orthologs that modern chimpanzees retain, we postulate that this explanation is questionable. First, human populations retain homologues of these same chimpanzee MHC proteins in relatively high frequencies, yet humans, with only very limited exceptions, do not appear naturally resistant to HIV-1 induced immunodeficiency. Second, the analysis presented by de Groot et al. (based upon use of Tajima's “D”, a statistical test for the action of positive selection) suggests that these genes have evolved neutrally. There is no support for positive selection on these chimpanzee loci, although MHC genes in other species have been documented to show molecular level selection (Hughes and Nei, (1988) Nature 335:167-170; Hughes and Nei, (1989) Proc. Natl. Acad. Sci. U.S.A. 86:958-962). Chimpanzee resistance to HIV-1 progression is unlikely to be conferred by the MHC alleles that remain in present day chimpanzee populations.

As detailed above, the changes we identified in chimpanzee ICAM 1, in particular, appear likely to modulate dimerization of chimpanzee ICAM 1. As ICAM 1-mediated cell adhesion functions (such as those exploited by HIV-1) are dependent upon binding to ligand, and as such binding has been shown to be influenced by the state of ICAM 1 dimerization, we propose that binding of chimpanzee ICAM 1 to its ligands is not blocked, but rather modulated, thus altering the cell adhesion functions needed by HIV-1, perhaps reducing viral infectivity.

### Example 23

### Two-Step Screening Process

We used a two-step screening process as a rigorous filter to narrow in on other genes responsible for chimpanzee disease resistance. Firstly, we restricted our search to those genes whose expression pattern changes after experimental HIV infection of human cells. Secondly, we screened this subset for genes that had undergone positive selection.

Several groups have reported in the literature investigations of the altered pattern of gene expression that results from infection of human cells in vitro. Each group has used different cell lines and experimental protocols, thus, although some overlap exists in results for all these studies, each investigation has also yielded a unique set of genes. Because of the large number of affected genes in such studies (in one study 3% of genes of T cells were affected), many investigators select small subsets of genes to characterize more completely; for example, Scheuring et al. (1998 AIDS 12: 563-570). selected 12 differentially expressed bands and described 4 host genes. Ryo et al. (1999, FEBS Letters 462(1-2):182-186) found 142 differentially expressed genes by SAGE analysis (minimum 5-fold difference in expression), of which they selected 53 that matched known genes and concluded that the genes whose expression was up-regulated by infection played a role in accelerated HIV replication and those down-regulated played a role in host cell defense. They subsequently sequenced and identified 13 cDNA fragments and observed coordinated expression of certain genes (Ryo et al. 2000 AIDS Res. Hum. Retroviruses 16: 995-1005). Corbeil et al. (2001 Genome Res 11: 1198-204) examined 6800 specific genes over 8 time points in a T-cell line to follow expression of genes involved in mitochondrial function and integrity, DNA repair, and apoptosis, but these authors as well as others caution that levels of key genes vary at different time points after infection. Vahey et al. (2003 AIDS Res. & Hum. Retroviruses 19: 369-387) used high density arrays of 5600 cellular genes from cells infected in vitro and also saw temporal patterns of coordinated expression of many genes. Su et al. (2002 Oncogene 21: 3592-602) examined differential gene expression in astrocytes infected with HIV-1. Two groups have been examining potential resistance mechanisms. Simm et al. (2001 Gene 269: 93-101) report eleven genes expressed differentially after HIV-1 inoculation of HIV-1 resistant vs. susceptible T cell lines, of which 5 are novel genes. Krasnoselskaya et al. (2002 AIDS Res. Hum. Retroviruses 18: 591-604) looked at gene expression differences between NF90-expressing cells (which are able to inhibit viral replication) vs. control cells and found 90 genes that had 4-fold or greater changes in expression, many having to do with interferon response.

We developed a method to select a subset of genes differentially expressed upon infection by HIV. We randomly chose genes reported by these others to be up or down regulated after HIV infection of human cells and designed primers to them. We obtained chimpanzee blood (Buckshire Labs, PA) and isolated mRNA. RT-PCR amplified chimpanzee homologs of the human genes. We determined the DNA sequence of each amplicon. We then performed pairwise Ka/Ks comparison of chimpanzee amplicon sequence vs. the homologous human sequence by means of EG's ATP software. Analysis was performed both upon complete coding regions, as well as on sliding windows (composed of smaller sections of the protein-coding region), in order to facilitate identification of small regions of these genes that have been positively selected. Candidate genes with elevated Ka/Ks ratios were amplified and sequenced from multiple chimpanzee and human individuals, in order to ascertain the degree of genetic heterogeneity that exists in the two species for these loci.

The efficacy of this two step process was demonstrated: of 100 chimpanzee genes we examined, only four showed the signature of positive selection. Thus, although the collection of genes whose expression patterns were altered as a result of immunodeficiency virus infection was extensive, we were able to narrow our search to four genes/proteins.

### Example 24

### CD98 Heavy Chain (GenBank J03569)

CD98 is a heterodimeric transmembrane glycoprotein (Rintoul et al. 2002). CD98 is a highly conserved protein, expressed nearly ubiquitously among cell types. The high level of evolutionary conservation observed among mammalian CD98 homologs makes even more striking the observation that CD98 has been positively selected between humans and chimpanzees. The positively selected portion of the coding sequence (approx. 730 bp in the heavy chain) shows a Ka/Ks ratio=1.7. (As is often the case, the full-length comparisons of CD98 between human and chimpanzee display a Ka/Ks ratio<1. Full-length comparisons frequently mask the signature of positive selection because the ‘noise’ of synonymous substitutions throughout the full coding sequence overwhelms the signal of positive selection in those cases when only a short portion of the sequence has been adaptively altered.)

CD98 has been linked (Rintoul et al. 2002) to cellular activation; evidence suggests that CD98 activates a tyrosine kinase-controlled signal transduction pathway (Warren et al. 1996) There is also evidence that CD98 regulates intracellular calcium concentrations through a Na⁺/Ca2⁺ exchanger (Michalak et al. 1986).

Strong evidence links CD98 to control of the inflammatory process (Rintoul et al. 2002). Intriguingly, Rintoul et al. (2002) state that “compelling evidence exists for a connection between CD98 and virus-induced cell fusion”. Ito et al. (1996) and Ohgimoto et al. (1995) have shown that antibodies to CD98 promote cell fusion that is induced by the gp160 envelope glycoprotein of HIV. The link to inflammatory processes and to virus-induced (and HIV-induced cell fusion, in particular) is significant. ICAMs are well known agents of the inflammatory response, and their part in HIV-induced cell fusion is well documented (Castilletti et al. 1995; Ott et al. 1997; Fortin et al. 1999). Thus the positively selected chimpanzee ICAMs participate with positively selected chimpanzee CD98 to effect HIV resistance.

### Example 25

### p44 (GenBank NM_{—}006417)

Two alleles were detected in chimpanzees (alleles A & B). Human to chimpanzee full-length comparisons gave Ka/Ks ratios of 0.42 for allele A and 0.45 for allele B. However, examination of exon 2 of the chimpanzee and human homologs revealed that this portion of the gene had been positively selected.

The protein p44 was discovered by Shimizu et al. (1985). These authors infected chimpanzees with non-A, non-B hepatitis (hepatitis C) and identified p44 as a protein that was expressed upon infection. For several years, p44 was a marker of hepatitis C infection, until the virus was cloned in 1989 and direct virus diagnostic techniques became available. Although chimpanzees have been used as a model for human hepatitis C, it has been well-documented that HCV-infected chimpanzees are refractory to the hepatic damage that often occurs in HCV-infected humans, perhaps due to lower levels of viral replication (Lanford et al. 1991). p44 is a member of the family of alpha/beta interferon inducible genes and thought to be a mediator of the antiviral activities of interferon induced by double-stranded RNA replicative intermediates (Kitamura et al. 1994). As HIV infection is characterized by a double-stranded RNA replicative intermediate, it was not surprising to find in Vahey et al.'s study (2003) on genes differentially expressed upon HIV infection, that p44 is listed among the hundreds of genes reported. However, while infection with hepatitis B virus does induce p44 expression, infection by the hepatitis G virus, which also is expected to replicate via a double-stranded RNA intermediate, does not induce expression of p44 (Shimizu et al. 2001). This positively selected protein, which is up-regulated after infection by both hepatitis C and HIV-1, is clearly of interest.

### Example 26

### IFN-β56K (GenBank M24594)

The positively selected portion of the coding sequence (approx. 1245 bp) shows a Ka/Ks ratio=2.5. Strikingly, for this protein, even the full-length comparison of between the human and chimpanzee homologs displays a Ka/Ks ratio greater than one (1.3)

IFN-β56k is a 56-kilodalton protein that plays a role in the control of protein synthesis. Generally, protein synthesis is initiated when eIF4F, eIF4G, and eIF4E and eIF3 work in concert to bring together ribosomes with messenger RNA. Many viruses usurp the host protein synthesis “machinery” to stop production of host proteins and instead produce virus-encoded proteins. Two HIV-1 encoded proteins appear to play a role in redirecting protein synthesis to HIV-encoded proteins. HIV protease has been shown to cleave eIF4GI (but not II), resulting in inhibition of cap-dependent mRNA translation while protein synthesis using non-capped mRNAs with internal ribosome entry sites (such as HIV mRNAs) continues or is even stimulated (Alvarez et al. 2003). HIV Vpr has been shown to act on a number of host cell functions, including enhancing expression of viral mRNAs. Vpr interacts directly with eIF3f, one of the twelve subunits of eIF3. When IFN-β56K is present, it binds to another of the subunits of eIF3 (eIF3e) and stops protein translation. IFN-β56K likely represents a host protein that is expressed during virus infection as part of a general antiviral interferon-mediated response.

In vitro, no mRNA encoding IFN-β56k is detectable in cells in the absence of treatment with interferon or dsRNA. After the addition of interferon or dsRNA, the amount of IFN-β56K mRNA increases; it has been reported to be the most abundant interferon-induced mRNA among the over one hundred INF-induced mRNAs measured (Der et al. 1998). IFN-β56K is inducible by interferons alpha, beta, and gamma, by virus infection (HIV, hepatitis C, Sendai virus, vesicular stomatitis virus, encephalomyocarditis virus, and cytomegalovirus) or by the presence of dsRNA.

Guo and Sen (2000) have characterized IFN-β56K extensively. The IFN-β56K protein has eight tetratricopeptide motifs; such motifs are generally associated with mediation of protein-protein interactions. Upon induction of expression of the IFN-β56K gene by the presence of interferon, IFI-56pK is present in the cytoplasm and eIF3e is located in the nucleus.

Upon the interaction of HIV Vpr with eIF3f, the latter translocates into the nucleus. Upon the interaction of IFN-β56K with eIF3e, the latter translocates into the cytoplasm.

### Example 27

### Staf50 (GenBank X82200)

This protein has been shown to be induced by both type I and type II human interferons (Tissot and Mechti 1995), and importantly, Staf50 has been shown to down-regulate transcription of the long terminal repeat of HIV-1 (Tissot and Mechti 1995). Thus, in addition to the fact that this protein is upregulated after HIV-1 infection, and the fact that it has been positively selected in HIV-resistant chimpanzees, this protein also plays a role on regulation of HIV-1 infection.

As is reported to be the case for IFN-β56K (and perhaps for p44), Staf50 appears to be part of a general antiviral response, mediated by the interferons. Chang and Laimins (2000) demonstrated by microarray analysis that the regulation of Staf50 is altered as a result of infection by the human papillomavirus type 31. Like p44 and IFN-β56K (Patzwahl et al. 2001), Staf50 has been shown to be upregulated in the chimpanzee liver after hepatitis C infection (Bigger et al. 2001).

Staf50 is the human homolog of mouse Rpt-1, which is known to negatively regulate the gene that codes for the IL-2 receptor (Bigger et al. 2001).

Although the foregoing invention has been described in some detail by way of illustration and example for purposes of clarity and understanding, it will be apparent to those of ordinary skill in the art that certain changes and modifications can be practiced. Therefore, the description and examples should not be construed as limiting the scope of the invention, which is delineated by the appended claims.

What is claimed is:

1. A method to identify an agent which may modulate resistance to HIV-1-mediated disease, comprising contacting at least one agent to be tested with a cell comprising human ICAM-1, and detecting the cell's resistance to HIV-1 viral replication, propagation, or function, wherein an agent is identified by its ability to increase the cell's resistance to HIV-1 viral replication, propagation, or function.

2. The method of claim 1, wherein the increased resistance to HIV-1 viral replication, propagation, or function is measured relative to that of a cell transfected with an effective amount of at least one of the following: a mutant human ICAM-1 comprising one or more of the following mutations to human ICAM-1: L18Q, K29D, P45G, R49W, E171Q wherein the mutant ICAM-1 is otherwise identical to human ICAM-1; and a primate ICAM-1.

3. The method of claim 1, wherein the human ICAM-1 sequence is SEQ ID NO:3.

4. The method of claim 2, wherein the primate ICAM-1 is a chimpanzee ICAM-1 comprising SEQ ID NO:85.

5. The method of claim 1, wherein the resistance to viral replication or propagation is demonstrated by reduction of HIV-1 expression in HIV-1 infected cells.

6. The method of claim 1, wherein the resistance to viral replication or propagation is a result of increased dimerization of two ICAM-1 polypeptides in the cell.

7. The method of claim 1, wherein the resistance to viral replication or propagation is a result of decreased dimerization of two ICAM-1 polypeptides in the cell.

8. The method of claim 1, wherein resistance to viral replication, propagation, or function is determined by measurement of virus-mediated cellular pathogenesis, cell to cell infectivity, virus-mediated cell fusion, virus-mediated syncytia formation, HIV-1 expression by the cell, inflammatory response suppression, and virus budding rate.

9. The method of claim 1, wherein the agent is a small molecule.

10. A human mutant ICAM-1 polypeptide comprising one or more of the following mutations to human ICAM-1: L18Q, K29D, P45G, R49W, E171Q, wherein the mutant ICAM-1 is otherwise identical to human ICAM-1, wherein said polypeptide confers increased resistance to HIV-1 viral replication, propagation, or function in a human cell.

11. A human cell comprising heterologous DNA the human mutant ICAM-1 polypeptide of claim 10; and a primate ICAM-1.

12. The composition of claim 11, wherein the primate ICAM-1 is a chimpanzee ICAM-1 comprising SEQ ID NO:85.

13. A method for inhibiting HIV-1 viral replication, propagation, or function in a human subject by ICAM-1 gene therapy, comprising the steps of: parenterally administering to a human subject at least one of the following: a viral vector comprising a mutant ICAM-1 comprising one or more of the following mutations: L 18Q, K29D, P45G, R49W, E171Q, and a viral vector comprising a non-human primate ICAM-1, allowing said ICAM-1 protein to be expressed from said gene in said subject in an amount sufficient to provide for inhibiting HIV-1 viral replication, propagation, or function in the human subject.

14. The method of claim 13, wherein increased resistance to AIDS comprises inhibition of production of HIV-1 in the subject.

15. The method of claim 13, wherein the primate ICAM-1 is a chimpanzee ICAM-1.

16. A method for inhibiting HIV-1 viral replication, propagation, or function in a human subject by ICAM-1 gene therapy, comprising the steps of: transfection of at least a portion of the subject's white blood cells with at least one of the following: a viral vector comprising a mutant ICAM-1 comprising one or more of the following mutations: L18Q, K29D, P45G, R49W, E 171Q, and a viral vector comprising a non-human primate ICAM-1, allowing said ICAM-1 protein to be expressed from at least a portion of the transfected white blood cells, in an amount sufficient to provide for inhibiting HIV-1 viral replication, propagation, or function in the human subject.

17. The method of claim 16, wherein the primate ICAM-1 is a chimpanzee ICAM-1.

18. The method of claim 16, wherein at least a portion of the subject's white blood cells are removed from the subject prior to transfection and returned to the subject post-transfection.

19. A method to treat an HIV-1 infection in a human subject, comprising administering a pharmaceutically effective amount of an agent which increases the human subject's resistance to HIV-1 viral replication, propagation, or function by modulating the function of human ICAM-1.

20. The method of claim 19, wherein the modulation of the function of human ICAM-1 results in resistance to HIV-1 viral replication, propagation, or function that is substantially similar to that provided by at least one of the following: a mutant human ICAM-1 comprising one or more of the following mutations to human ICAM-1: L18Q, K29D, P45G, R49W, E171Q wherein the mutant ICAM-1 is otherwise identical to human ICAM-1; and a primate ICAM-1.

21. The method of claim 19, wherein the resistance to viral replication or propagation is reduction of HIV-1 expression in HIV-1 infected cells.

22. The method of claim 19, wherein the resistance to viral replication or propagation is a result of increased dimerization of two ICAM-1 polypeptides.

23. The method of claim 19, wherein the resistance to viral replication or propagation is a result of decreased dimerization of two ICAM-1 polypeptides.

24. The method of claim 19, wherein resistance to viral replication, propagation, or function is determined by measurement of virus-mediated cellular pathogenesis, cell to cell infectivity, virus-mediated cell fusion, virus-mediated syncytia formation, HIV-1 expression by the cell, inflammatory response suppression, and virus budding rate.

25. The method of claim 19, wherein the agent is a small molecule.

26. The method of claim 20, wherein the primate ICAM-1 is chimpanzee ICAM-1.

27. A small molecule modulator of human ICAM-1 identified by the method of claim 1.

28. A method to identify an agent which may modulate resistance to HIV-1-mediated disease, comprising contacting at least one agent to be tested with human ICAM-1, and detecting the increased or decreased dimerization of human ICAM-1, wherein an agent is identified by its ability to increase or decrease dimerization of the human ICAM-1 subunits whereby said increased or decreased dimerization of human ICAM-1 modulates resistance to HIV-1 modulated disease.

29. A method to identify an agent which may modulate resistance to HIV-1-mediated disease, comprising contacting at least one agent to be tested with human ICAM-1, and detecting a change in ICAM-1 mediated cell to cell signaling, wherein an agent is identified by its ability to increase or decrease ICAM-1 mediated cell to cell signaling whereby said ICAM-1 mediated cell to cell signaling modulates resistance to HIV-1 modulated disease.


SEQUENCE LISTING (84 sequences)

SEQ ID NO: 1 - DNA, 1518 bp, Homo sapiens
cagacatctgtgtccccctcaaaagtcatcctgccccggggaggctccgtgctggtgacatgcagcacctcctgtgaccagcccaagttgttgggcatagagaccccgttgcctaaaaaggagttgctcctgcctgggaacaaccggaaggtgtatgaactgagcaatgtgcaagaagatagccaaccaatgtgctattcaaactgccctgatgggcagtcaacagctaaaaccttcctcaccgtgtactggactccagaacgggtggaactggcacccctcccctcttggcagccagtgggcaagaaccttaccctacgctgccaggtggagggtggggcaccccgggccaacctcaccgtggtgctgctccgtggggagaaggagctgaaacgggagccagctgtgggggagcccgctgaggtcacgaccacggtgctggtgaggagagatcaccatggagccaatttctcgtgccgcactgaactggacctgcggccccaagggctggagctgtttgagaacacctcggccccctaccagctccagacctttgtcctgccagcgactcccccacaacttgtcagcccccgggtcctagaggtggacacgcaggggaccgtggtctgttccctggacgggctgttcccagtctcggaggcccaggtccacctggcactgggggaccagaggttgaaccccacagtcacctatggcaacgactccttctcggccaaggcctcagtcagtgtgaccgcagaggacgagggcacccagcggctgacgtgtgcagtaatactggggaaccagagccaggagacactgcagacagtgaccatctacagctttccggcgcccaacgtgattctgacgaagccagaggtctcagaagggaccgaggtgacagtgaagtgtgaggcccaccctagagccaaggtgacgctgaatggggttccagcccagccactgggcccgagggcccagctcctgctgaaggccaccccagaggacaacgggcgcagcttctcctgctctgcaaccctggaggtggccggccagcttatacacaagaaccagacccgggagcttcgtgtcctgtatggcccccgactggacgagagggattgtccgggaaactggacgtggccagaaaattcccagcagactccaatgtgccaggcttgggggaacccattgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagtcataatgggcactgcaggcctcagcacgtacctctataaccgccagcggaagatcaagaaatacagactacaacaggcccaaaaagggacccccatgaaaccgaacacacaagccacgcctccctga

SEQ ID NO: 2 - DNA, 1518 bp, Homo Sapiens; identical to SEQ ID NO: 1
  CDS (1)..(1518)

SEQ ID NO: 3 - protein, 505 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTREVTVNVLSPRYEIVIITVVAAAVIMGTAGLSTYLYNRQRKIKKYRLQQAQKGTPMKPNTQATPP

SEQ ID NO: 4 - DNA, 1515 bp, Gorilla gorilla
cagacatctgtgtcccccccaaaagtcatcctgccccggggaggctccgtgctggtgacatgcagcacctcctgtgaccagcccaccttgttgggcatagagaccccgttgcctaaaaaggagttgctcctgcttgggaacaaccagaaggtgtatgaactgagcaatgtgcaagaagatagccaaccaatgtgttattcaaactgccctgatgggcagtcaacagctaaaaccttcctcaccgtgtactggactccagaacgggtggaactggcacccctcccctcttggcagccagtgggcaaggaccttaccctacgctgccaggtggagggtggggcaccccgggccaacctcatcgtggtgctgctccgtggggaggaggagctgaaacgggagccagctgtgggggagcccgccgaggtcacgaccacggtgccggtggagaaagatcaccatggagccaatttcttgtgccgcactgaactggacctgcggccccaagggctgaagctgtttgagaacacctcggccccctaccagctccaaacctttgtcctgccagcgactcccccacaacttgtcagccctcgggtcctagaggtggacacgcaggggactgtggtctgttccctggacgggctgttcccagtctcggaggcccaggtccacctggcactgggggaccagaggttgaaccccacagtcacctatggcaacgactccttctcagccaaggcctcagtcagtgtgaccgcagaggacgagggcacccagtggctgacgtgtgcagtaatactggggacccagagccaggagacactgcagacagtgaccatctacagctttccggcacccaacgtgattctgacgaagccagaggtctcagaagggaccgaggtgacagtgaagtgtgaggcccaccctagagccaaggtgacactgaatggggttccagcccagccaccgggcccgaggacccagttcctgctgaaggccaccccagaggacaacgggcgcagcttctcctgctctgcaaccctggaggtggccggccagcttatacacaagaaccagacccgggagcttcgtgtcctgtatggcccccgactggatgagagggattgtccgggaaactggacgtggccagaaaattcccagcagactccaatgtgccaggcttgggggaacccattgcccgagctcaagtgtctaaaggatggcactttcccactgcccgtcggggaatcagtgactgtcactcgagatcttgagggcacctacctctgtcgggccaggagcactcaaggggaggtcacccgcgaggtgaccgtgaatgtgctctccccccggtatgagtttgtcatcatcgctgtggtagcagccgcagtcataatgggcactgcaggcctcagcacgtacctctataaccgccagcggaagatcaggaaatacagactacaacaggctcaaaaagggacccccatgaaaccgaacacacaagccacgcctccc

SEQ ID NO: 5 - DNA, 1515 bp, Pongo pygmaeus
cacacatctgtgtcctccgccaacgtcttcctgccccggggaggctccgtgctagtgaattgcagcacctcctgtgaccagcccaccttgttgggcatagagaccccgttgcctaaaaaggagttgctcccgggtgggaacaactggaagatgtatgaactgagcaatgtgcaagaagatagccaaccaatgtgctattcaaactgccctgatgggcagtcagcagctaaaaccttcctcaccgtgtactggactccagaacgggtggaactggcacccctcccctcttggcagccagtgggcaagaaccttaccctacgctgccaggtggagggtggggcaccccgggccaacctcaccgtggtattgctccgtggggaggaggagctgagccggcagccagcggtgggggagcccgccgaggtcacggccacggtgctggcgaggaaagatgaccacggagccaatttctcgtgccgcactgaactggacctgcggccccaagggctggagctgtttgagaacacctcggccccccaccagctccaaacctttgtcctgccagcgactcccccacaacttgtcagcccccgggtcctagaggtggacacgcaggggaccgtggtctgttccctggacgggctgttcccagtctcggaggcccaggtccacttggcactgggggaccagaggttgaaccccacagtcacctatggcgtcgactccctctcggccaaggcctcagtcagtgtgaccgcagaggaggagggcacccagtggctgtggtgtgcagtgatactgaggaaccagagccaggagacacggcagacagtgaccatctacagctttcctgcacccaacgtgactctgatgaagccagaggtctcagaagggaccgaggtgatagtgaagtgtgaggcccaccctgcagccaacgtgacgctgaatggggttccagcccagccgccgggcccgagggcccagttcctgctgaaggccaccccagaggacaacgggcgcagcttctcctgctctgcaaccctggaggtggccggccagcttatacacaagaaccagacccgggagcttcgagtcctgtatggcccccgactggacgagagggattgtccgggaaactggacgtggccagaaaactcccagcagactccaatgtgccaggcttgggggaaccccttgcccgagctcaagtgtctaaaggatggcactttcccactgcccatcggggaatcagtgactgtcactcgagatcttgagggcacctacctctgtcgggccaggagcactcaaggggaggtcacccgcgaggtgaccgtgaatgtgctctccccccggtatgagattgtcatcatcactgtggtagcagccgcagccatactgggcactgcaggcctcagcacgtacctctataaccgccagcggaagatcaggatatacagactacaacaggctcaaaaagggacccccatgaaaccaaacacacaaaccacgcctccc

SEQ ID NO: 6 - protein, 505 aa, Homo sapiens; identical to SEQ ID NO: 3

SEQ ID NO: 7 - protein, 254 aa, Homo sapiens
SDEKVFEVHVRPKKLAVEPKGSLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 8 - protein, 518 aa, Homo sapiens
QEFLLRVEPQNPVLSAGGSLFVNCSTDCPSSEKIALETSLSKELVASGMGWAAFNLSNVTGNSRILCSVYCNGSQITGSSNITVYGLPERVELAPLPPWQPVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 9 - DNA, 1212 bp, Homo sapiens
atgagtgactccaaggaaccaagactgcagcagctgggcctcctggaggaggaacagctgagaggccttggattccgacagactcgaggatacaagagcttagcagggtgtcttggccatggtcccctggtgctgcaactcctctccttcacgctcttggctgggctccttgtccaagtgtccaaggtccccagctccataagtcaggaacaatccaggcaagacgcgatctaccagaacctgacccagcttaaagctgcagtgggtgagctctcagagaaatccaagctgcaggagatctaccaggagctgacccagctgaaggctgcagtgggtgagcttccagagaaatctaagctgcaggagatctaccaggagctgacccggctgaaggctgcagtgggtgagcttccagagaaatctaagctgcaggagatctaccaggagctgacctggctgaaggctgcagtgggtgagcttccagagaaatctaagatgcaggagatctaccaggagctgactcggctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgacccagctgaaggctgcagtggaacgcctgtgccacccctgtccctgggaatggacattcttccaaggaaactgttacttcatgtctaactcccagcggaactggcacgactccatcaccgcctgcaaagaagtgggggcccagctcgtcgtaatcaaaagtgctgaggagcagaacttcctacagctgcagtcttccagaagtaaccgcttcacctggatgggactttcagatctaaatcaggaaggcacgtggcaatgggtggacggctcacctctgttgcccagcttcaagcagtattggaacagaggagagcccaacaacgttggggaggaagactgcgcggaatttagtggcaatggctggaacgacgacaaatgtaatcttgccaaattctggatctgcaaaaagtccgcagcctcctgctccagggatgaagaacagtttctttctccagcccctgccaccccaaacccccctcctgcg

SEQ ID NO: 10 - DNA, 1212 bp, Pan troglodytes
atgagtgactccaaggaaccaagactgcagcagctgggcctcctggaggaggaacagctgagaggccttggattccgacagactcgaggctacaagagcttagcagggtgtcttggccatggtcccctggtgctgcaactcctctccttcacgctcttggctgggctccttgtccaagtgtccaaggtccccagctccataagtcaggaagaatccaggcaagacgtgatctaccagaacctgacccagcttaaagctgcagtgggtgagctctcagagaaatccaagctgcaggagatctaccaggagctgacccagctgaaggctgcagtgggtgagcttccagagaaatctaagcagcaggagatctaccaggagctgacccggctgaaggctgcagtgggtgagcttccagagaaatctaagatgcaggagatctaccaggagctgactcggctgaaggctgcagtgggtgagcttccagagaaatctaagatgcaggagatctaccaggagctgactcggctgaaggctgcagtgggtgagcttccagagaaatctaagcagcaggagatctaccaggagctgacccagctgaaggctgcagtgggtgagcttccagagaaatctaagcagcaggagatctaccaggagctgacccagctgaaggctgcagtgggtgagcttccagagaaatctaagcagcaggagatctaccaggagctgacccggctgaaggctgcagtggaacgcctgtgccgccgctgcccctgggaatggacattcttccaaggaaactgttacttcatgtctaactcccagcggaactggcacgactccatcactgcctgcaaagaagtgggggcccagctcgtcgtaatcaaaagtgctgaggagcagaacttcctacagctgcagtcttccagaagtaaccgcttcacctggatgggactttcagatctaaatgaggaaggcatgtggcaatgggtggacggctcacctctgttgcccagcttcaaccagtaytggaacagaggagagcccaacaacgttggggaggaagactgcgcggaatttagtggcaatggctggaatgacgacaaatgtaatcttgccaaattctggatctgcaaaaagtccgcagcctcctgctccagggatgaagaacagtttctttctccagcccctgccaccccaaacccccctcctgcg

SEQ ID NO: 11 - DNA, 1212 bp, Gorilla gorilla
atgagtgactccaaggaaccaagactgcagcagctgggcctcctggaggaggaacagctgagaggccttggattccgacagactcgaggctacaagagcttagcagggtgtcttggccatggtcccctggtgctgcaactcctctccttcacgctcttggctgcgctccttgtccaagtgtccaaggtccccagctccataagtcaggaacaatccaggcaagacgcgatctaccagaacctgacccagtttaaagctgcagtgggtgagctctcagagaaatccaagctgcaggagatctatcaggagctgacccagctgaaggctgcagtgggtgagcttccagagaaatctaagcagcaggagatctaccaggagctgagccagctgaaggctgcagtgggtgagcttccagagaaatctaagcagcaggagatctaccaggagctgacccggctgaaggctgcagtgggtgagcttccagagaaatctaagcagcaggagatctaccaggagctgacccggctgaaggctgcagtgggtgagcttccagagaaatctaagcagcaggagatctaccaggagctgagccagctgaaggctgcagtgggtgagcttccagagaaatctaagcagcaggagatctaccaggagctgagccagctgaaggctgcagtgggtgagcttccagagaaatctaagcagcaggagatctaccaggagctgacccagctgaaggctgcagtggaacgcctgtgccgccgctgcccctgggaatggacattcttccaaggaaactgttacttcatgtctaactcccagcggaactggcacgactccatcaccgcctgccaagaagtgggggcccagctcgtcgtaatcaaaagtgctgaggagcagaacttcctacagctgcagtcttccagaagtaaccgcttcacctggatgggactttcagatctaaatcatgaaggcacgtggcaatgggtggacggctcacctctgttgcccagcttcgagcagtattggaacagaggagagcccaacaacgttggggaggaagactgcgcggaatttagtggcaatggctggaacgatgacaaatgtaatcttgccaaattctggatctgcaaaaagtctgcagcctcctgctccagggatgaagaacagtttctttctccagcctctgccaccccaaacccccctcctgcg

SEQ ID NO: 12 - protein, 105 aa, Pan troglodytes
SLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAAWSLHP

SEQ ID NO: 13 - protein, 121 aa, Pan troglodytes
SLQCYNCPNPTADCKTAVNCSSDFDACLITKAGLQVYNKCWKLEHCNFKDLTTRLRENELTYYCCKKDLCNFNEQLENGGNEQLENGGNEQLENGGTSLSEKTVLLRVTPFLAAAAWSLHP

SEQ ID NO: 14 - DNA, 5140 bp, Homo sapiens
ctccagacctacccagaaagatgcccggatggatcctgcagctccgtggcttttctgggaagcagcggcccctgctctcaagagaccctggctcctgatggtggccccaaggttgccagctggtgctagggactcaggacagtttcccagaaaaggccaagcgggcagcccctccaggggccgggtgaggaagctggggggtgcggaggccacactgggtccctgaaccccctgcttggttacagtgcagctcctcaagtccacagacgtgggccggcacagcctcctgtacctgaaggaaatcggccgtggctggttcgggaaggtgttcctgggggaggtgaactctggcatcagcagtgcccaggtggtggtgaaggagctgcaggctagtgccagcgtgcaggagcagatgcagttcctggaggaggtgcagccctacagggccctgaagcacagcaacctgctccagtgcctggcccagtgcgccgaggtgacgccctacctgctggtgatggagttctgcccactgggggacctcaagggctacctgcggagctgccgggtggcggagtccatggctcccgacccccggaccctgcagcgcatggcctgtgaggtggcctgtggcgtcctgcaccttcatcgcaacaatttcgtgcacagcgacctggccctgcggaactgcctgctcacggctgacctgacggtgaagattggtgactatggcctggctcactgcaagtacagagaggactacttcgtgactgccgaccagctgtgggtgcctctgcgctggatcgcgccagagctggtggacgaggtgcatagcaacctgctcgtcgtggaccagaccaagagcgggaatgtgtggtccctgggcgtgaccatctgggagctctttgagctgggcacgcagccctatccccagcactcggaccagcaggtgctggcgtacacggtccgggagcagcagctcaagctgcccaagccccagctgcagctgaccctgtcggaccgctggtacgaggtgatgcagttctgctggctgcagcccgagcagcggcccacagccgaggaggtgcacctgctgctgtcctacctgtgtgccaagggcgccaccgaagcagaggaggagtttgaacggcgctggcgctctctgcggcccggcgggggcggcgtggggcccgggcccggtgcggcggggcccatgctgggcggcgtggtggagctcgccgctgcctcgtccttcccgctgctggagcagttcgcgggcgacggcttccacgcggacggcgacgacgtgctgacggtgaccgagaccagccgaggcctcaattttgagtacaagtgggaggcgggccgcggcgcggaggccttcccggccacgctgagccctggccgcaccgcacgcctgcaggagctgtgcgcccccgacggcgcgcccccgggcgtggttccggtgctcagcgcgcacagcccgtcgctgggcagcgagtacttcatccgcctagaggaggccgcacccgccgccggccacgaccctgactgcgccggctgcgcccccagtccacctgccaccgcggaccaggacgacgactctgacggcagcaccgccgcctcgctggccatggagccgctgctgggccacgggccacccgtcgacgtcccctggggccgcggcgaccactaccctcgcagaagcttggcgcgggacccgctctgcccctcacgctctccctcgccctcggcggggcccctgagtctggcggagggaggagcggaggatgcagactggggcgtggccgccttctgtcctgccttcttcgaggacccactgggcacgtcccctttggggagctcaggggcgcccccgctgccgctgactggcgaggatgagctagaggaggtgggagcgcggagggccgcccagcgcgggcactggcgctccaacgtgtcagccaacaacaacagcggcagccgctgtccagagtcctgggaccccgtctctgcgggctgccacgctgagggctgccccagtccaaagcagaccccacgggcctcccccgagccggggtaccctggagagcctctgcttgggctccaggcagcctctgcccaggagccaggctgctgccccggcctccctcatctatgctctgcccagggcctggcacctgctccctgcctggttacaccctcctggacagagacagccagtagtgggggtgaccacccgcaggcagagcccaagcttgccacggaggctgagggcactaccggaccccgcctgccccttccttccgtcccctccccatcccaggagggagccccacttccctcggaggaggccagtgcccccgacgcccctgatgccctgcctgactctcccacgcctgctactggtggcgaggtgtctgccatcaagctggcttctgccctgaatggcagcagcagctctcccgaggtggaggcacccagcagtgaggatgaggacacggctgaggccacctcaggcatcttcaccgacacgtccagcgacggcctgcaggccaggaggccggatgtggtgccagccttccgctctctgcagaagcaggtggggacccccgactccctggactccctggacatcccgtcctcagccagtgatggtggctatgaggtcttcagcccgtcggccactggcccctctggagggcagccgcgagcgctggacagtggctatgacaccgagaactatgagtcccctgagtttgtgctcaaggaggcgcaggaagggtgtgagccccaggcctttgcggagctggcctcagagggtgagggccccgggcccgagacacggctctccacctccctcagtggcctcaacgagaagaatccctaccgagactctgcctacttctcagacctcgaggctgaggccgaggccacctcaggcccagagaagaagtgcggcggggaccgagcccccgggccagagctgggcctgccgagcactgggcagccgtctgagcaggtctgtctcaggcctggggtttccggggaggcacaaggctctggccccggggaggtgctgcccccactgctgcagcttgaagggtcctccccagagcccagcacctgcccctcgggcctggtcccagagcctccggagccccaaggcccagccaaggtgcggcctgggcccagccccagctgctcccagtttttcctgctgaccccggttccgctgagatcagaaggcaacagctctgagttccaggggcccccaggactgttgtcagggccggccccacaaaagcggatggggggcccaggcacccccagagccccactccgcctggctctgcccggcctccctgcggccttggagggccggccggaggaggaggaggaggacagtgaggacagcgacgagtctgacgaggagctccgctgctacagcgtccaggagcctagcgaggacagcgaagaggaggcgccggcggtgcccgtggtggtggctgagagccagagcgcgcgcaacctgcgcagcctgctcaagatgcccagcctgctgtccgagaccttctgcgaggacctggaacgcaagaagaaggccgtgtccttcttcgacgacgtcaccgtctacctctttgaccaggaaagccccacccgggagctcggggagcccttcccgggcgccaaggaatcgccccctacgttccttagggggagccccggctctcccagcgcccccaaccggccgcagcaggctgatggctccccaaatggctccacagcggaagagggtggtgggttcgcgtgggacgacgacttcccgctgatgacggccaaggcagccttcgccatggccctagacccggccgcacccgccccggctgcgcccacgcccacgcccgctcccttctcgcgcttcacggtgtcgcccgcgcccacgtcccgcttctccatcacgcacgtgtctgactcggacgccgagtccaagagaggacctgaagctggtgccgggggtgagagtaaagaggcttgagacctgggcagctcctgcccctcaaggctggcgtcaccggagcccctgccaggcagcagcgaggatggtgaccgagaaggtggggaccacgtcctggtggctgttggcagcagattcaggtgcctctgccccacgcggtgtcctggagaagcccgtgggatgagaggccctggatggtagatcggccatgctccgccccagaggcagaattcgtctgggcttttaggcttgctgctagcccctgggggcgcctggagccacagtgggtgtctgtacacacatacacactcaaaaggggccagtgcccctgggcacggcggcccccaccctctgccctgcctgcctggcctcggaggacccgcatgccccatccggcagctcctccggtgtgctcacaggacacttaaaccaggacgaggcatggccccgagacactggcaggtttgtgagcctcttcccaccccctgtgcccccacccttgcctggttcctggtggctcagggcaaggagtggccctgggcgcccgtgtcggtcctgtttccgctgcccttatctcaaagtccgtggctgtttccccttcactgactcagctagacccgtaagcccacccttcccacagggaacaggctgctcccacctgggtcccgctgtggccacggtgggcagcccaaaagatcaggggtggaggggcttccaggctgtactcctgccccgtgggccccgttctagaggtgcccttggcaggaccgtgcaggcagctcccctctgtggggcagtatctggtcctgtgccccagctgccaaaggagagtgggggccatgccccgcagtcagtgttggggggctcctgcctacagggagagggatggtggggaaggggtggagctgggggcagggcagcacagggaatatttttgtaactaactaactgctgtggttggagcgaatggaagttgggtgattttaagttattgttgccaaagagatgtaaagtttattgttgcttcgcagggggatttgttttgtgttttgtttgaggcttagaacgctggtgcaatgttttcttgttccttgttttttaagagaaatgaagctaagaaaaaag

SEQ ID NO: 15 - DNA, 5140 bp, Homo sapiens; identical to SEQ ID NO: 14
  CDS (413)..(4036)

SEQ ID NO: 16 - protein, 1207 aa, Homo sapiens
MQFLEEVQPYRALKHSNLLQCLAQCAEVTPYLLVMEFCPLGDLKGYLRSCRVAESMAPDPRTLQRMACEVACGVLHLHRNNFVHSDLALRNCLLTADLTVKIGDYGLAHCKYREDYFVTADQLWVPLRWIAPELVDEVHSNLLVVDQTKSGNVWSLGVTIWELFELGTQPYPQHSDQQVLAYTVREQQLKLPKPQLQLTLSDRWYEVMQFCWLQPEQRPTAEEVHLLLSYLCAKGATEAEEEFERRWRSLRPGGGGVGPGPGAAGPMLGGVVELAAASSFPLLEQFAGDGFHADGDDVLTVTETSRGLNFEYKWEAGRGAEAFPATLSPGRTARLQELCAPDGAPPGVVPVLSAHSPSLGSEYFIRLEEAAPAAGHDPDCAGCAPSPPATADQDDDSDGSTAASLAMEPLLGHGPPVDVPWGRGDHYPRRSLARDPLCPSRSPSPSAGPLSLAEGGAEDADWGVAAFCPAFFEDPLGTSPLGSSGAPPLPLTGEDELEEVGARRAAQRGHWRSNVSANNNSGSRCPESWDPVSAGCHAEGCPSPKQTPRASPEPGYPGEPLLGLQAASAQEPGCCPGLPHLCSAQGLAPAPCLVTPSWTETASSGGDHPQAEPKLATEAEGTTGPRLPLPSVPSPSQEGAPLPSEEASAPDAPDALPDSPTPATGGEVSAIKLASALNGSSSSPEVEAPSSEDEDTAEATSGIFTDTSSDGLQARRPDVVPAFRSLQKQVGTPDSLDSLDIPSSASDGGYEVFSPSATGPSGGQPRALDSGYDTENYESPEFVLKEAQEGCEPQAFAELASEGEGPGPETRLSTSLSGLNEKNPYRDSAYFSDLEAEAEATSGPEKKCGGDRAPGPELGLPSTGQPSEQVCLRPGVSGEAQGSGPGEVLPPLLQLEGSSPEPSTCPSGLVPEPPEPQGPAKVRPGPSPSCSQFFLLTPVPLRSEGNSSEFQGPPGLLSGPAPQKRMGGPGTPRAPLRLALPGLPAALEGRPEEEEEDSEDSDESDEELRCYSVQEPSEDSEEEAPAVPVVVAESQSARNLRSLLKMPSLLSETFCEDLERKKKAVSFFDDVTVYLFDQESPTRELGEPFPGAKESPPTFLRGSPGSPSAPNRPQQADGSPNGSTAEEGGGFAWDDDFPLMTAKAAFAMALDPAAPAPAAPTPTPAPFSRFTVSPAPTSRFSITHVSDSDAESKRGPEAGAGGESKEA

SEQ ID NO: 17 - DNA, 1803 bp, Pan troglodytes
gctccctgcctggttacaccctcctggacagagacagccggtagtgggggtgaccacccgcaggcagagcccaagcttgccacggaggctgagggcactgccggaccctgtctgccccttccttccgtcccctccccatcccaggagggagccccacttccctcggaggaggccagtgcccctgacgcccctgatgccctgcctgactctcccatgcctgctactggtggcgaggtgtctgccatcaagctggcttctgtcctgaatggcagcagcagctctcccgaggtggaggcacccagcagcgaggatgaggacacggctgaggccacctcaggcatcttcaccgacacgtccagcgacggcctgcaggccgagaggctggatgtggtgccagccttccgctctctgcagaagcaggtggggacccccgactccctggactccctggacatcccatcctcagccagtgatggtggctatgaggtcttcagcccgtcggccactggcccctctggagggcagccccgagcgctggacagtggctatgacaccgagaactatgagtcccctgagtttgtgctcaaggaggcgcaggaagggtgtgagccccaggcctttgaggagctggcctcagagggtgagggccccggccccgggcccgagacgcggctctccacctccctcagtggcctcaacgagaagaatccctaccgagactctgcctacttctcagacctggaggctgaggccgaggccgaggccacctcaggcccagagaagaagtgcggcggggaccaagcccccgggccagagctggacctgccgagcactgggcagccgtctgagcaggtctccctcaggcctggggtttccggggaggcacaaggctctggccccggggaggtgctgcccccactgctgcggcttgaaggatcctccccagagcccagcacctgcccctcgggcctggtcccagagcctccggagccccaaggcccagccgaggtgcggcctgggcccagccccagctgctcccagtttttcctgctgaccccggttccgctgagatcagaaggcaacagctctgagttccaggggcccccaggactgttgtcagggccggccccacaaaagcggatggggggcctaggcacccccagagccccactccgcctggctctgcccggcctccctgcggccttggagggccggccggaggaggaggaggaggacagtgaggacagcggcgagtctgacgaggagctccgctgctacagcgtccaggagcctagcgaggacagcgaagaggaggcgccggcggtgcccgtggtggtggctgagagccagagcgcgcgcaacctgcgcagcctgctcaagatgcccagcctgctgtccgaggccttctgcgaggacctggaacgcaagaagaaggccgtgtccttcttcgacgacgtcaccgtctacctctttgaccaggaaagccccacctgggagctcggggagcccttcccgggcgccaaggaatcgccccccacgttccttagggggagccccggctctcccagcgcccccaaccggccgcagcaggctgatggctccccaaatggctccacagcggaagagggtggtgggttcgcgtgggacgacgacttcccgctgatgccggccaaggcagccttcgccatggccctagacccggccgcacccgccccggctgcgcccacgcccgctcccttctcgcgcttcacggtgtcgcccgcgcccacgtccacgtcccgcttctccatcacgcacgtgtct

SEQ ID NO: 18 - DNA, 1785 bp, Gorilla gorilla
gctccctgcctggttacaccctcctggacagagacagacggtagtgggggtgaccacccgcaggcagagcccaagcttgccacggaggctgagggcactgccggaccccgcctgccccttccttccgtcccctccccatcccaggagggagccccacttccctcggaggaggccagtgcccccgacgcccctgatgccctgcctgactcgcccacgcctgctactggtggcgaggtgtctgccaccaagctggcttccgccctgaatggcagcagcagctctcccgaggtggaggcacccagcagtgaggatgaggacacggctgaggcaacctcaggcatcttcaccgacacgtccagcgacggcctgcaggccgagaggcaggatgtggtgccagccttccactctctgcagaagcaggtggggacccccgactccctggactccctggacatcccgtcctcagccagtgatggtggctatgaggtcttcagcccgtcggccacgggcccctctggagggcagccccgagcgctggacagtggctatgacaccgagaactatgagtcccctgagtttgtgctcaaggaggcgcaggaagggtgtgagccccaggcctttgcggagctggcctcagagggcgagggccccgggcccgagacgcggctctccacctccctcagtggcctcaacgagaagaatccctaccgagattctgcctacttctcagacctggaggctgaggccgaggctacctcaggcccagagaagaagtgcggtggggaccaagcccccgggccagagctgggcctgccgagcactgggcagccgtctgagcaggtctccctcagtcctggggtttccgtggaggcacaaggctctggccccggggaggtgctgcccccactgctgcggcttgaagggtcctccccagagcccagcacctgcccctcgggcctggtcccagagcctccggagccccaaggcccagccgaggtgcggcctgggcccagccccagctgctcccagtttttcctgctgaccccggttccgctgagatcagaaggcaacagctctgagttccaggggcccccaggactgttgtcagggccggccccacaaaagcggatggggggcccaggcacccccagagccccacaccgcctggctctgcccggcctccctgcggccttggagggccggccggaggaggaggaggaggacagtgaggacagcgacgagtctgacgaggagctccgctgctacagcgtccaggagcctagcgaggacagcgaagaggaggcgccggcggtgcccgtggtggtggctgagagccagagcgcgcgcaacctgcgcagcctgctcaagatgcccagcctgctgtccgaggccttctgcgaggacctggaacgcaagaagaaggccgtgtccttcttcgacgacgtcaccgtctacctctttgaccaggaaagccccacccgggagctcggggagcccttcccgggcgccaaggaatcgccccccacgttccttagggggagccccggctcttccagcgcccccaaccggccgcagcaggctgatggctccccaaatggctccacagcggaagagggtggtgggttcgcgtgggacgacgacttcccgctgatgccggccaaggcagccttcgccatggccctagacccggccgcacccgccccggctgcgcccacgcccgctcccttctcgcgcttcacggtgtcgcccgcgcccacgtcccgcttctccatcacgcacgtgtct

SEQ ID NO: 19 - DNA, 24 bp, Pan troglodytes
ggtgagggccccggccccgggccc

SEQ ID NO: 20 - DNA, 18 bp, Homo sapiens
ggtgagggccccgggccc

SEQ ID NO: 21 - DNA, 18 bp, Gorilla gorilla
ggcgagggccccgggccc

SEQ ID NO: 22 - DNA, 24 bp, Pan troglodytes
ctggaggctgaggccgaggccgag

SEQ ID NO: 23 - DNA, 18 bp, Homo sapiens
ctcgaggctgaggccgag

SEQ ID NO: 24 - DNA, 18 bp, Gorilla gorilla
ctggaggctgaggccgag

SEQ ID NO: 25 - DNA, 18 bp, Pan troglodytes
cccacgcccgctcccttc

SEQ ID NO: 26 - DNA, 24 bp, Homo sapiens
cccacgcccacgcccgctcccttc

SEQ ID NO: 27 - DNA, 18 bp, Gorilla gorilla
cccacgcccgctcccttc

SEQ ID NO: 28 - DNA, 24 bp, Pan troglodytes
cccacgtccacgtcccgcttctcc

SEQ ID NO: 29 - DNA, 18 bp, Homo sapiens
cccacgtcccgcttctcc

SEQ ID NO: 30 - DNA, 18 bp, Gorilla gorilla
cccacgtcccgcttctcc

SEQ ID NO: 31 - DNA, 1335 bp, Pan troglodytes
atggcagtgacaactcgtttgacatggttgcatgaaaagatcctgcaaaatcattttggagggaagcggcttagccttctctataagggtagtgtccatggattccataatggagttttgcttgacagatgttgtaatcaagggcctactctaacagtgatttatagtgaagatcatattattggagcatatgcagaagagggttaccaggmaagaaagtatgcttccatcatcctttttgcacttcaagagactaaaatttcagaatggaaactaggactatatacaccagaaacactgttttgttgtgacgttgcaaaatataactccccaactaatttccagatagatggaagaaatagaaaagtgattatggacttaaagacaatggaaaatcttggacttgctcaaaattgtactatctctattcaggattatgaagtttttcgatgcgaagattcactggacgaaagaaagataaaaggggtcattgagctcaggaagagcttactgtctgccttgagaacttatgaaccatatggatccctggttcaacaaatacgaattctgctgctgggtccaattggagctgggaagtctagctttttcaactcagtgaggtctgttttccaagggcatgtaacgcatcaggctttggtgggcactaatacaactgggatatctgagaagtataggacatactctattagagacgggaaagatggcaaatacctgccatttattctgtgtgactcactggggctgagtgagaaagaaggcggcctgtgcatggatgacatatcctacatcttgaacggtaacattcgtgatagataccagtttaatcccatggaatcaatcaaattaaatcatcatgactacattgattccccatcgctgaaggacagaattcattgtgtggcatttgtatttgatgccagctctattgaatacttctcctctcagatgatagtaaagatcaaaagaattcgaagggagttggtaaacgctggtgtggtacatgtggctttgctcactcatgtggatagcatggatctgattacaaaaggtgaccttatagaaatagagagatgtgtgcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatag

SEQ ID NO: 32 - DNA, 1335 bp, Pan troglodytes; identical to SEQ ID NO: 31
  CDS (1)..(1335)
  misc_feature (71)..(71): Xaa = Glu or Ala

SEQ ID NO: 33 - protein, 444 aa, Pan troglodytes
  misc_feature (71)..(71): The ′Xaa′ at location 71 stands for Glu, or Ala.
MAVTTRLTWLHEKILQNHFGGKRLSLLYKGSVHGFHNGVLLDRCCNQGPTLTVIYSEDHIIGAYAEEGYQXRKYASIILFALQETKISEWKLGLYTPETLFCCDVAKYNSPTNFQIDGRNRKVIMDLKTMENLGLAQNCTISIQDYEVFRCEDSLDERKIKGVIELRKSLLSALRTYEPYGSLVQQIRILLLGPIGAGKSSFFNSVRSVFQGHVTHQALVGTNTTGISEKYRTYSIRDGKDGKYLPFILCDSLGLSEKEGGLCMDDISYILNGNIRDRYQFNPMESIKLNHHDYIDSPSLKDRIHCVAFVFDASSIEYFSSQMIVKIKRIRRELVNAGVVHVALLTHVDSMDLITKGDLIEIERCVPVRSKLEEVQRKLGFALSDISVVSNYSSEWELDPVKDVLILSALRRMLWAADDFLEDLPFEQIGNLREEIINCAQGKK

SEQ ID NO: 34 - DNA, 1335 bp, Homo sapiens
atggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctcctcctgggtccaattggagctcccaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcaaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatag

SEQ ID NO: 35 - DNA, 1335 bp, Homo sapiens; identical to SEQ ID NO: 34
  CDS (1)..(1335)

SEQ ID NO: 36 - protein, 444 aa, Homo sapiens
MAVTTRLTWLHEKILQNHFGGKRLSLLYKGSVHGFRNGVLLDRCCNQGPTLTVIYSEDHIIGAYAEESYQEGKYASIILFALQDTKISEWKLGLCTPETLFCCDVTKYNSPTNFQIDGRNRKVIMDLKTMENLGLAQNCTISIQDYEVFRCEDSLDERKIKGVIELRKSLLSALRTYEPYGSLVQQIRILLLGPIGAPKSSFFNSVRSVFQGHVTHQALVGTNTTGISEKYRTYSIRDGKDGKYLPFILCDSLGLSEKEGGLCRDDIFYILNGNIRDRYQFNPMESIKLNHHDYIDSPSLKDRIHCVAFVFDASSIQYFSSQMIVKIKRIQRELVNAGVVHVALLTHVDSMDLITKGDLIEIERCEPVRSKLEEVQRKLGFALSDISVVSNYSSEWELDPVKDVLILSALRRMLWAADDFLEDLPFEQIGNLREEIINCAQGKK

SEQ ID NO: 37 - DNA, 1590 bp, Pan troglodytes
atgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgctggcgggtgccgtggtcataatcgtgcgggcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggcccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcatttttggctgagtggcaaaacatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcactggtgcagctggagtttgtctcaggcaaggctcctgacttccttcttgccggctcaacttctccgactctaccagctgatgctcttcaccctgccagggacccctgttttcagctacggggatgagattggcctggatgcggctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctga

SEQ ID NO: 38 - DNA, 1590 bp, Pan troglodytes; identical to SEQ ID NO: 37
  CDS (1)..(1590)

SEQ ID NO: 39 - protein, 529 aa, Pan troglodytes
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNHWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 40 - DNA, 1861 bp, Homo sapiens
ggggggggagatgcagtagccgaaaactgcgcggaggcacgagaggccggggagagcgttctgggtccgagggtccaggtaggggttgagccaccatctgaccgcaagctgcgtcgtgtcgccttctctgcaggcaccatgagccaggacaccgaggtggatatgaaggaggtggagctgaatgagttagagcccgagaagcagccgatgaacgcggcgtctggggcggccatgtccctggcggaagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtgataatcgtgcgagcgccgcgttgtcgcgagctaccggcgcagaagtggtggcacacgggccccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgacaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtggagacaggctcttgattgcggggactaactcctccgaccttcagcagatcctgagcctactcgaatccaacaaagacttgctgttgactagctcatacctgtctgattctggttctactccccagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctcttttcagctacggggatgagattggcctggatgcagctgcccttcctccacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcctgagctgggacgcctgaaactggagcctcacgaagggctgctgctccgcttcccctacgcggcctgacctcagcctgacatggacccactacccttctcctttccttcccaggccctttggcttctgattttttttctcttttttaaaacaaacaaacaaactgttgcagattatgagtgaaccccaaatagggtgtttt

SEQ ID NO: 41 - DNA, 1861 bp, Homo sapiens; identical to SEQ ID NO: 40
  CDS (139)..(1728)

SEQ ID NO: 42 - protein, 529 aa, Homo sapiens
MSQDTEVDMKEVELNELEPEKQPMNAASGAAMSLAEAEKNGLVKIKVAEDEAEAAAAAKFTGLSKEELLKVAGSPGWVRTRWALLLLFWLGWLGMLAGAVVIIVRAPRCRELPAQKWWHTGPLYRIGDLQAFQGHGAGNLAGLKGRLDYLSSLKVKGLVLGPIHKNQKDDVAQTDLLQIDPNFGSKEDFDSLLQSAKKKSIRVILDLTPNYRGDNSWFSTQVDTVATKVKDALEFWLQAGVDGFQVRDIENLKDASSFLAEWQNITKGFSGDRLLIAGTNSSDLQQILSLLESNKDLLLTSSYLSDSGSTPQHTKSLVTQYLNATGNRWCSWSLSQARLLTSFLPAQLLRLYQLMLFTLPGTPLFSYGDEIGLDAAALPPQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPPELGRLKLEPHEGLLLRFPYAA

SEQ ID NO: 43 - DNA, 1437 bp, Pan troglodytes
atgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacaatgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacataccattttctttgcttcccctaaggcaggctgtccgtttaaatccggacaatggatatatgaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattagaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagagttttcgaaaaatgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattagcaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgacttcgagaactctgtgagacaaggtccttag

SEQ ID NO: 44 - DNA, 1437 bp, Pan troglodytes; identical to SEQ ID NO: 43
  CDS (1)..(1437)

SEQ ID NO: 45 - protein, 478 aa, Pan troglodytes
MSTNGDDHQVKDSLEQLRCHFTWELSIDDDEMPDLENRVLDQIEFLDTKYNVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHIPFSLLPLRQAVRLNPDNGYMKVLLALKLQDEGQEAEGEKYIEEALANMSSQTYVFRYAAKFYRRKGSVDKALELLEKALQETPTSVLLHHQIGLCYKAQMIQIKEATKGQPRGQNREKLDKMIRSAIFHFESAVEKKPTFEVAHLDLARMYIEAGNHRKAEESFRKMLCMKPVVEETMQDIHFHYGRFQEFQKKSDVNAIIHYLKAIKIEQASLARDKSINSLKKLVLRKLRRKALDLESLSLLGFVYKLEGNMNEALEYYERALRLAADFENSVRQGP

SEQ ID NO: 46 - DNA, 1642 bp, Homo sapiens
ccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaagaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttctactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctataaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatatt

SEQ ID NO: 47 - DNA, 1642 bp, Homo sapiens; identical to SEQ ID NO: 46
  CDS (65)..(1501)

SEQ ID NO: 48 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFYYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 49 - DNA, 1341 bp, Pan troglodytes
atggatttctcagtaaaggtagacatagagaaggaggtgacctgccccatctgcctggagctcctgacagaacctctgagcctagattgtggccacagcttctgccaagcctgcatcactacaaagatcaaggagtcagtgatcatctcaagaggggaaagcagctgtcctgtgtgtcagaccagattccagcctgggaacctccgacctaatcggcatctggccaacatagttgagagagtcaaagaggtcaagatgagcccacaggaggggcagaagagagatgtctgtgagcaccatggaaaaaaactccagatcttctgtaaggaggatggaaaagtcatttgctgggtttgtgaactgtctccggaacaccaaggtcaccaaacattccgcataaacgaggtggtcaaggaatgtcaggaaaagctgcaggtagccctgcagaggctgataaaggaggatcaagaggctgagaagctggaagatgacatcagacaagagagaaccgcctggaagaattatatccagatcgagagacagaagattctgaaagggttcaatgaaatgagagtcatcttggacaatgaggagcagagagagctgcaaaagctggaggaaggtgaggtgaatgtgctggataacctggcagcagctacagaccagctggtccagcagaggcaggatgccagcacgctcatctcagatctccagcggaggttgaggggatcgtcagtagagatgctgcaggatgtgattgacgtcatgaaaaggagtgaaagctggacattgaagaagccaaaatctgtttccaagaaactaaagagtgtattccgagtaccagatctgagtgggatgctgcaagttcttaaagagctgacagatgtccagtactactgggtggacgtgatgctgaatccaggcagtgccacttcgaatgttgctatttctgtggatcagagacaagtgaaaactgtacgcacctgcacatttaagaattcaaatccatgtgatttttctgcttttggtgtcttcggctgccaatatttctcttcggggaaatattactgggaagtagatgtgtctggaaagattgcctggatcctgggcgtacacagtaaaataagtagtctgaataaaaggaagagctctgggtttgcttttgatccaagtgtaaattattcaaaagtttactccaaatatagacctcaatatggctactgggttataggattacagaatacatgtgaatataatgcttttgaggactcctcctcttctgatcccaaggttttgactctctttatggctgtgctccctgtcgtattggggttttcctag

SEQ ID NO: 50 - DNA, 1341 bp, Pan troglodytes; identical to SEQ ID NO: 49

SEQ ID NO: 51 - DNA, 2811 bp, Homo sapiens
gaattcggcacgagctcttctcccctgattcaagactcctctgctttggactgaagcactgcaggagtttgtgaccaagaacttcaagagtcaagacagaaggaagccaagggagcagtgcaatggatttctcagtaaaggtagacatagagaaggaggtgacctgccccatctgcctggagctcctgacagaacctctgagcctagattgtggccacagcttctgccaagcctgcatcactgcaaagatcaaggagtcagtgatcatctcaagaggggaaagcagctgtcctgtgtgtcagaccagattccagcctgggaacctccgacctaatcggcatctggccaacatagttgagagagtcaaagaggtcaagatgagcccacaggaggggcagaagagagatgtctgtgagcaccatggaaaaaaactccagatcttctgtaaggaggatggaaaagtcatttgctgggtttgtgaactgtctcaggaacaccaaggtcaccaaacattccgcataaacgaggtggtcaaggaatgtcaggaaaagctgcaggtagccctgcagaggctgataaaggaggatcaagaggctgagaagctggaagatgacatcagacaagagagaaccgcctggaagatcgagagacagaagattctgaaagggttcaatgaaatgagagtcatcttggacaatgaggagcagagagagctgcaaaagctggaggaaggtgaggtgaatgtgctggacaacctggcagcagctacagaccagctggtccagcagaggcaggatgccagcacgctcatctcagatctccagcggaggttgacgggatcgtcagtagagatgctgcaggatgtgattgacgtcatgaaaaggagtgaaagctggacattgaagaagccaaaatctgtttccaagaaactaaagagtgtattccgagtaccagatctgagtgggatgctgcaagttcttaaagagctgacagatgtccagtactactgggtggacgtgatgctgaatccaggcagtgccacttcgaatgttgctatttctgtggatcagagacaagtgaaaactgtacgcacctgcacatttaagaattcaaatccatgtgatttttctgcttttggtgtcttcggctgccaatatttctcttcggggaaatattactgggaagtagatgtgtctggaaagattgcctggatcctgggcgtacacagtaaaataagtagtctgaataaaaggaagagctctgggtttgcttttgatccaagtgtaaattattcaaaagtttactccagatatagacctcaatatggctactgggttataggattacagaatacatgtgaatataatgcttttgaggactcctcctcttctgatcccaaggttttgactctctttatggctgtgctccctgtcgtattggggttttcctagactatgaggcaggcattgtctcatttttcaatgtcacaaaccacggacgactcatctacaagttctctggatgtcgcttttctcgacctgcttatccgtatttcaatccttggaactgcctagtccccatgactgtgtgcccaccgagctcctgagtgttctcattcctttacccacttctgcatagtagcccttctgtgagactcagattctgcacctgagttcatctctactgagaccatctcttcctttctttccccttcttttacttagaatgtctttgtattcatttgctagggcttccatagcaaagcatcatagattgctgatttaaactgtaattgtattgccgtactgtgggctgaaatcccaaatctagattccagcagagttggttctttctgaggtctgcaaggaagggctctgttccatgcctctctccttggcttgtagaaggcatcttgtccctatgactcttcacattgtctttatgtacatctctgtgcccaagttttccctttttattaagacaccagtcatactggcctcagggcccaccgctaatgccttaatgaaatcattttaacattatattgtgtacaaagaccttatttccaaataagataatatttggaggtattgggaataaaatttgaggaaggcgatttcactcataacaatcttaccctttcttgcaagagatgcttgtacattattttcctaataccttggtttcactagtagtaaacattattattttttttatatttgcaaaggaaacatatctaatccttcctatagaaagaacagtattgctgtaattccttttcttttcttcctcatttcctctgccccttaaaagattgaagaaagagaaacttgtcaactcatatccacgttatctagcaaagtcataagaatctatcactaagtaatgtatccttcagaatgtgttggtttaccagtgacaccccatattcatcacaaaattaaagcaagaagtccatagtaatttatttgctaatagtggatttttaatgctcagagtttctgaggtcaaattttatcttttcacttacaagctctatgatcttaaataatttacttaatgtattttggtgtattttcctcaaattaatattggtgttcaagactatatctaattcctctgatcactttgagaaacaaacttttattaaatgtaaggcacttttctatgaattttaaatataaaaataaatattgttctgattattactgaaaagatgtcagccatttcaatgtcttgggaaacaattttttgtttttgttctgttttctttttgcttcaataaaacaatagctggctctaaaaaaaaaa

SEQ ID NO: 52 - DNA, 2811 bp, Homo sapiens; identical to SEQ ID NO: 51
  CDS (123)..(1451)

SEQ ID NO: 53 - protein, 442 aa, Homo sapiens
MDFSVKVDIEKEVTCPICLELLTEPLSLDCGHSFCQACITAKIKESVIISRGESSCPVCQTRFQPGNLRPNRHLANIVERVKEVKMSPQEGQKRDVCEHHGKKLQIFCKEDGKVICWVCELSQEHQGHQTFRINEVVKECQEKLQVALQRLIKEDQEAEKLEDDIRQERTAWKIERQKILKGFNEMRVILDNEEQRELQKLEEGEVNVLDNLAAATDQLVQQRQDASTLISDLQRRLTGSSVEMLQDVIDVMKRSESWTLKKPKSVSKKLKSVFRVPDLSGMLQVLKELTDVQYYWVDVMLNPGSATSNVAISVDQRQVKTVRTCTFKNSNPCDFSAFGVFGCQYFSSGKYYWEVDVSGKIAWILGVHSKISSLNKRKSSGFAFDPSVNYSKVYSRYRPQYGYWVIGLQNTCEYNAFEDSSSSDPKVLTLFMAVLPVVLGFS

SEQ ID NO: 54 - DNA, 825 bp, Homo sapiens
atgtcctctttcggttacaggaccctgactgtggccctcttcaccctgatctgctgtccaggatcggatgagaaggtattcgaggtacacgtgaggccaaagaagctggcggttgagcccaaagggtccctcgaggtcaactgcagcaccacctgtaaccagcctgaagtgggtggtctggagacctctctagataagattctgctggacgaacaggctcagtggaaacattacttggtctcaaacatctcccatgacacggtcctccaatgccacttcacctgctccgggaagcaggagtcaatgaattccaacgtcagcgtgtaccagcctccaaggcaggtcatcctgacactgcaacccactttggtggctgtgggcaagtccttcaccattgagtgcagggtgcccaccgtggagcccctggacagcctcaccctcttcctgttccgtggcaatgagactctgcactatgagaccttcgggaaggcagcccctgctccgcaggaggccacagccacattcaacagcacggctgacagagaggatggccaccgcaacttctcctgcctggctgtgctggacttgatgtctcgcggtggcaacatctttcacaaacactcagccccgaagatgttggagatctatgagcctgtgtcggacagccagatggtcatcatagtcacggtggtgtcggtgttgctgtccctgttcgtgacatctgtcctgctctgcttcatcttcggccagcacttgcgccagcagcggatgggcacctacggggtgcgagcggcttggaggaggctgccccaggccttccggcca

SEQ ID NO: 55 - DNA, 825 bp, Homo sapiens; identical to SEQ ID NO: 54
  CDS (1)..(825)

SEQ ID NO: 56 - protein, 275 aa, Homo sapiens
MSSFGYRTLTVALFTLICCPGSDEKVFEVHVRPKKLAVEPKGSLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATATFNSTADREDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 57 - DNA, 825 bp, Pan troglodytes
atgtcctctttcagttacaggaccctgactgtggccctcttcgccctgatctgctgtccaggatcggatgagaaggtattcgaggtacacgtgaggccaaagaagctggcggttgagcccaaagggtccctcaaggtcaactgcagcaccacctgtaaccagcctgaagtgggtggtctggagacctctctagataagattctgctggacgaacaggctcagtggaaacattacttggtctcaaacatctcccatgacacggtcctccaatgccacttcacctgctccgggaagcaggagtcaatgaattccaacgtcagcgtgtaccagcctccaaggcaggtcatcctgacactgcaacccactttggtggctgtgggcaagtccttcaccattgagtgcagggtgcccaccgtggagcccctggacagcctcaccctcttcctgttccgtggcaatgagactctgcactatgagaccttcgggaaggcagcccctgctccgcaggaggccacagtcacattcaacagcacggctgacagagacgatggccaccgcaacttctcctgcctggctgtgctggacttgatgtctcgcggtggcaacatctttcacaaacactcagccccgaagatgttggagatctatgagcctgtgtcggacagccagatggtcatcatagtcacggtggtgtcggtgttgctgtccctgttcgtgacatctgtcctgctctgcttcatcttcggccagcacttgcgccagcagcggatgggcacctacggggtgcgagcggcttggaggaggctgccccaggccttccggcca

SEQ ID NO: 58 - DNA, 825 bp, Pan troglodytes; identical to SEQ ID NO: 57
  CDS (1)..(825)

SEQ ID NO: 59 - protein, 275 aa, Pan troglodytes
MSSFSYRTLTVALFALICCPGSDEKVFEVHVRPKKLAVEPKGSLKVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHYETFGKAAPAPQEATVTFNSTADRDDGHRNFSCLAVLDLMSRGGNIFHKHSAPKMLEIYEPVSDSQMVIIVTVVSVLLSLFVTSVLLCFIFGQHLRQQRMGTYGVRAAWRRLPQAFRP

SEQ ID NO: 60 - DNA, 825 bp, Gorilla gorilla
atgtcctctttcggttacaggacactgactgtggccctcttcgccctgatctgctgtccaggatctgatgagaaggtatttgaggtacacgtgaggccaaagaagctggcggttgagcccaaagcgtccctcgaggtcaactgcagcaccacctgtaaccagcctgaagtgggtggtctggagacctctctagataagattctgctggacgaacaggctcagtggaaacattacttggtctcaaacatctcccatgacacggtcctccaatgccacttcacctgctccgggaagcaggagtcaatgaattccaacgtcagcgtgtaccagcctccaaggcaggtcatcctgacactgcaacccactttggtggctgtgggcaagtccttcaccattgagtgcagggtgcccaccgtggagcccctggacagcctcaccctcttcctgttccgtggcaatgagactctgcacaatcagaccttcgggaaggcagcccctgctctgcaggaggccacagccacattcaacagcacggctgacagagaggatggccaccgcaacttctcctgcctggctgtgctggacttgatatctcgcggtggcaacatctttcaggaacactcagccccaaagatgttggagatctatgagcctgtgtcggacagccagatggtcatcatagtcacggtggtgtcggtgttgctgtccctgttcgtgacatctgtcctgctctgcttcatcttcggccagcacttgcgccagcagcggatgggcacctatggggtgcgagcggcttggaggaggctgccccaggccttccggcca

SEQ ID NO: 61 - DNA, 825 bp, Gorilla gorilla; identical to SEQ ID NO: 60
  CDS (1)..(825)

SEQ ID NO: 62 - protein, 275 aa, Gorilla gorilla
MSSFGYRTLTVALFALICCPGSDEKVFEVHVRPKKLAVEPKASLEVNCSTTCNQPEVGGLETSLDKILLDEQAQWKHYLVSNISHDTVLQCHFTCSGKQESMNSNVSVYQPPRQVILTLQPTLVAVGKSFTIECRVPTVEPLDSLTLFLFRGNETLHNQTFGKAAPALQEATATFNSTADREDGHRNFSCLAVLDLISRGGNIFQEHSAPKMLEIYEPVSDSQMVIIVTVVSVLLSLFVTSVLLCFIFGQHLRQQRMGTYGVRAAWRRLPQAFRP

SEQ ID NO: 63 - DNA, 762 bp, Macaca mulatta
tctgatgagaaggcattcgaggtacatatgaggctagagaagctgatagtaaagcccaaggagtccttcgaggtcaactgcagcaccacctgtaaccagcctgaagtgggtggtctggagacttctctaaataagattctgctgctcgaacagactcagtggaagcattacttgatctcaaacatctcccatgacacggtcctctggtgccacttcacctgctctgggaagcagaagtcaatgagttccaacgtcagcgtgtaccagcctccaaggcaggtcttcctcacactgcagcccacttgggtggccgtgggcaagtccttcaccatcgagtgcagggtgcccgccgtggagcccctggacagcctcaccctcagcctgctccgtggcagtgagactctgcacagtcagaccttcgggaaggcagcccctgccctgcaggaggccacagccacattcagcagcatggctcacagagaggacggccaccacaacttctcctgcctggctgtgctggacttgatgtctcgcggtggcgaagtcttctgcacacactcagccccgaagatgctggagatctatgagcccgtgccggacagccagatggtcatcatcgtcacagtggtgtcagtgttgctgttcctgttcgtgacatctgtcctgctctgcttcatcttcagccagcactggcgccagcggcggatgggcacctacggggtgcgagcggcttggaggaggctaccccaggccttccggcca

SEQ ID NO: 64 - DNA, 762 bp, Macaca mulatta; identical to SEQ ID NO: 63
  CDS (1)..(762)

SEQ ID NO: 65 - protein, 254 aa, Macaca mulatta
SDEKAFEVHMRLEKLIVKPKESFEVNCSTTCNQPEVGGLETSLNKILLLEQTQWKHYLISNISHDTVLWCHFTCSGKQKSMSSNVSVYQPPRQVFLTLQPTWVAVGKSFTIECRVPAVEPLDSLTLSLLRGSETLHSQTFGKAAPALQEATATFSSMAHREDGHHNFSCLAVLDLMSRGGEVFCTHSAPKMLEIYEPVPDSQMVIIVTVVSVLLFLFVTSVLLCFIFSQHWRQRRMGTYGVRAAWRRLPQAFRP

SEQ ID NO: 66 - DNA, 1608 bp, Pan troglodytes
agggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacaggctcccggagcgtgtggagctggcacccctgcctccttggcagcgggtgggccagaacttcaccctgcgctgccaagtggagggtgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccactgtgaacctcagcgagcccaccgcccctgaggggtccacagtgaccgtgagttgcatggctggggctcgagtccaggtcacgctggacggagttccggccgcggccccggggcagccagctcaacttcagctaaatgctaccgagagtgacgacagacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgacacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcacaaacggagcggcagttaccatgttagggaggagagcacctatctgcccctcacgtctatgcagccgacacaagcaatgggggaagaaccgtccagagctgag

SEQ ID NO: 67 - DNA, 1608 bp, Pan troglodytes; identical to SEQ ID NO: 66
  CDS (1)..(1608)

SEQ ID NO: 68 - protein, 536 aa, Pan troglodytes
RACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQRVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPTVNLSEPTAPEGSTVTVSCMAGARVQVTLDGVPAAAPGQPAQLQLNATESDDRRSFFCSATLEVDGEFLHRNSSVQLRVLYGPKIDRATCPQHLKWKDKTTHVLQCQARGNPYPELRCLKEGSSREVPVGIPFFVNVTHNGTYQCQASSSRGKYTLVVVMDIEAGSSHFVPVFVAVLLTLGVVTIVLALMYVFREHKRSGSYHVREESTYLPLTSMQPTQAMGEEPSRAE

SEQ ID NO: 69 - DNA, 1610 bp, Pan troglodytes
ccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacaggctcccggagcgtgtggagctggcacccctgcctccttggcagcgggtgggccagaacttcaccctgcgctgccaagtggagggtgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccactgtgaacctcagcgagcccaccgcccctgaggggtccacagtgaccgtgagttgcatggctggggctcgagtccaggtcacgctggacggagttccggccgcggccccggggcagccagctcaacttcagctaaatgctaccgagagtgacgacagacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgacacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcacaaacggagcggcagttaccatgttagggaggagagcacctatctgcccctcacgtctatgcagccgacagaagcaatgggggaagaaccgtccagagctgag

SEQ ID NO: 70 - DNA, 1610 bp, Pan troglodytes; identical to SEQ ID NO: 69
  CDS (3)..(1610)

SEQ ID NO: 71 - protein, 536 aa, Pan troglodytes
RACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQRVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPTVNLSEPTAPEGSTVTVSCMAGARVQVTLDGVPAAAPGQPAQLQLNATESDDRRSFFCSATLEVDGEFLHRNSSVQLRVLYGPKIDRATCPQHLKWKDKTTHVLQCQARGNPYPELRCLKEGSSREVPVGIPFFVNVTHNGTYQCQASSSRGKYTLVVVMDIEAGSSHFVPVFVAVLLTLGVVTIVLALMYVFREHKRSGSYHVREESTYLPLTSMQPTEAMGEEPSRAE

SEQ ID NO: 72 - DNA, 1605 bp, Gorilla gorilla
gcctgctggactctgctgctctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacaggctcccggagcgtgtggagctggcacccctgcctccttggcagccggtgggccagaacttcaccctgcgctgccaagtggagggtgggtcgccccggaccagcctcacggtggtgctgcttcgctgggaggaggagctgagccggcagcccgcagtggaggagccagcggaggtcactgcccctgtgctggccagcagaggcgaccatggagcccctttctcatgccgcacagaactggacatgcagccccaggggctgggactgttcgtgaacacctcagccccccgccagctccgaacctttgtcctgcccatgacccccccgcgcctcgtggccccccggttcttggaggtggaaacgtcgtggccggtggactgcaccctagacgggctttttccggcctcagaggcccaggtctacctggcgctgggggaccagatgctgaatgcgacagtcatgaaccacggggacacgctaacggccacagccacagccacggcgctcgcggatcaggagggtgcccgggagatcgtctgcaacgtgaccctagggggcgagagacgggaggcccgggagaacttgacgatctttagcttcctaggacccattgtgaacctcagcgagcccaccgcccctgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgacacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcacaaacggagcggcagttaccatgttagggaggagagcacctatctgcccctcacgtctatgcagccgacagaagcaatgggggaagaaccgtccagagctgag

SEQ ID NO: 73 - DNA, 1605 bp, Gorilla gorilla; identical to SEQ ID NO: 72
  CDS (1)..(1605)

SEQ ID NO: 74 - protein, 535 aa, Gorilla gorilla
ACWTLLLCCLLTPGVQGQEFLLRVEPQNPVLSAGGSLFVNCSTDCPSSEKIALETSLSKELVASGMGWAAFNLSNVTGNSRILCSVYCNGSQITGSSNITVYRLPERVELAPLPPWQPVGQNFTLRCQVEGGSPRTSLTVVLLRWEEELSRQPAVEEPAEVTAPVLASRGDHGAPFSCRTELDMQPQGLGLFVNTSAPRQLRTFVLPMTPPRLVAPRFLEVETSWPVDCTLDGLFPASEAQVYLALGDQMLNATVMNHGDTLTATATATALADQEGAREIVCNVTLGGERREARENLTIFSFLGPIVNLSEPTAPEGSTVTVSCMAGARVQVTLDGVPAAAPGQPAQLQLNATESDDGRSFFCSATLEVDGEFLHRNSSVQLRVLYGPKIDRATCPQHLKWKDKTTHVLQCQARGNPYPELRCLKEGSSREVPVGIPFFVNVTHNGTYQCQASSSRGKYTLVVVMDIEAGSSHFVPVFVAVLLTLGVVTIVLALMYVFREHKRSGSYHVREESTYLPLTSMQPTEAMGEEPSRAE

SEQ ID NO: 75 - DNA, 1614 bp, Homo sapiens
tggcccagggcctgctggactctgctggtctgctgtctgctgaccccaggtgtccaggggcaggagttccttttgcgggtggagccccagaaccctgtgctctctgctggagggtccctgtttgtgaactgcagtactgattgtcccagctctgagaaaatcgccttggagacgtccctatcaaaggagctggtggccagtggcatgggctgggcagccttcaatctcagcaacgtgactggcaacagtcggatcctctgctcagtgtactgcaatggctcccagataacaggctcctctaacatcaccgtgtacgggctcccggagcgtgtggagctggcacccctgcctccttggcagccggtgggccagaacttcaccctgcgctgccaagtggagggtgggtcgccccggaccagcctcacggtggtgctgcttcgctgggaggaggagctgagccggcagcccgcagtggaggagccagcggaggtcactgccactgtgctggccagcagagacgaccacggagcccctttctcatgccgcacagaactggacatgcagccccaggggctgggactgttcgtgaacacctcagccccccgccagctccgaacctttgtcctgcccgtgacccccccgcgcctcgtggccccccggttcttggaggtggaaacgtcgtggccggtggactgcaccctagacgggctttttccagcctcagaggcccaggtctacctggcgctgggggaccagatgctgaatgcgacagtcatgaaccacggggacacgctaacggccacagccacagccacggcgcgcgcggatcaggagggtgcccgggagatcgtctgcaacgtgaccctagggggcgagagacgggaggcccgggagaacttgacggtctttagcttcctaggacccattgtgaacctcagcgagcccaccgcccatgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttcttgcacaggaacagtagcgtccagctgcgagtcctgtatggtcccaaaattgaccgagccacatgcccccagcacttgaaatggaaagataaaacgagacacgtcctgcagtgccaagccaggggcaacccgtaccccgagctgcggtgtttgaaggaaggctccagccgggaggtgccggtggggatcccgttcttcgtcaacgtaacacataatggtacttatcagtgccaagcgtccagctcacgaggcaaatacaccctggtcgtggtgatggacattgaggctgggagctcccactttgtccccgtcttcgtggcggtgttactgaccctgggcgtggtgactatcgtactggccttaatgtacgtcttcagggagcaccaacggagcggcagttaccatgttagggaggagagcacctatctgcccctcacgtctatgcagccgacagaagcaatgggggaagaaccgtccagagctgag

SEQ ID NO: 76 - DNA, 1614 bp, Homo sapiens; identical to SEQ ID NO: 75
  CDS (1)..(1614)

SEQ ID NO: 77 - protein, 538 aa, Homo sapiens
WPRACWTLLVCCLLTPGVQGQEFLLRVEPQNPVLSAGGSLFVNCSTDCPSSEKIALETSLSKELVASGMGWAAFNLSNVTGNSRILCSVYCNGSQITGSSNITVYGLPERVELAPLPPWQPVGQNFTLRCQVEGGSPRTSLTVVLLRWEEELSRQPAVEEPAEVTATVLASRDDHGAPFSCRTELDMQPQGLGLFVNTSAPRQLRTFVLPVTPPRLVAPRFLEVETSWPVDCTLDGLFPASEAQVYLALGDQMLNATVMNHGDTLTATATATARADQEGAREIVCNVTLGGERREARENLTVFSFLGPIVNLSEPTAHEGSTVTVSCMAGARVQVTLDGVPAAAPGQPAQLQLNATESDDGRSFFCSATLEVDGEFLHRNSSVQLRVLYGPKIDRATCPQHLKWKDKTRHVLQCQARGNPYPELRCLKEGSSREVPVGIPFFVNVTHNGTYQCQASSSRGKYTLVVVMDIEAGSSHFVPVFVAVLLTLGVVTIVLALMYVFREHQRSGSYHVREESTYLPLTSMQPTEAMGEEPSRAE

SEQ ID NO: 78 - DNA, 1650 bp, pongo pygmaeus
gggcctgctggactctgctggtctgctgtctgctgaccccaggtgcccaggggcaggagttcctgctgcgggtggagccccagaaccctgtgctccctgctggagggtccctgttggtgaactgcagtactgattgtcccagctctaagaaaattgccttggagacgtccctatcaaaggagctggtggacaatggcatgggctgggcagccttctacctcagcaacgtgactggcaacagtaggatcctctgctcagtttactgcaatggctcccagataataggctcctctaacatcaccgtgtacaggctcccggagcgcgtggagctggcacccctgcctctttggcagccggtgggccagaacttcaccctgcgctgccaagtggagggtgggtcgccccggaccagcctcacggtggtgctgcttcgctgggaggaggagctgagccggcaacccgcagtggaagagccagcggaggtcactgccactgtgctggccagcagaggccaccacggagcccatttctcatgccgcacagaactggacatgcagccccaggggctgggactgttcgtgaacacctcagccccccgccagctccgaacctttgtcctgcccgtgacccccccgcgcctagtggctccccggttcttggaggcggaaacgtcgtggccggtggactgcaccctagatgggctttttccggcctcagaggcccaggtctacctggcgctgggggaccagatgctgaatgcgacagtcgtgaaccacggggacacgctgacggccacagccacagccatggcgcgcgcggatcaggagggtgcccaggagatcgtctgcaacgtgaccctagggggcgagagacgggaggcccgggagaacttgacggtctttagcttcctaggacccattctgaatctcagcgagcccagcgcccctgaggggtccacagtgaccgtgagttgcatggctggggctcgagtccaggtcacgctggacggagttccggccgcggccccggggcagccagctcaacttcagctaaatgctaccgagagtgacgacggacgcagcttcttctgcagtgccactctcgaggtggacggcgagttctttcacaggaacagtagcgtccagctgcgtgtcctgtatggtcccaaaattgaccgagccacatgcccccagcacttgaagtggaaagataaaacgagacacgtcctgcagtgccaagccaggggcaacccgcaccccgagctgcgatgtttgaaggaaggctccagccgggaggtgccggtggggatcccgttcttcgttaatgtaacacataatggtacttatcagtgccaagcgtccagctcacgaggcagatacaccctggtcgtggtgatggacattgaggctgggaactcccactttgtcctcgtcttcttggcggtgttagtgaccctgggcgtggtgactgtcgtagtggccttaatgtacgtcttcagggagcacaaacggagcggcaggtaccatgttaggcaggagagcacctctctgcccctcacgtctatgcagccgacagaggcaatgggggaagaaccgtccacagctgagtgacgctcggatccggggtcaaagttggcggggacttggctgt

SEQ ID NO: 79 - DNA, 1650 bp, Pongo pygmeaus; identical to SEQ ID NO: 78
  CDS (3)..(1649)

SEQ ID NO: 80 - protein, 535 aa, Pongo pygmeaus
ACWTLLVCCLLTPGAQGQEFLLRVEPQNPVLPAGGSLLVNCSTDCPSSKKIALETSLSKELVDNGMGWAAFYLSNVTGNSRILCSVYCNGSQIIGSSNITVYRLPERVELAPLPLWQPVGQNFTLRCQVEGGSPRTSLTVVLLRWEEELSRQPAVEEPAEVTATVLASRGHHGAHFSCRTELDMQPQGLGLFVNTSAPRQLRTFVLPVTPPRLVAPRFLEAETSWPVDCTLDGLFPASEAQVYLALGDQMLNATVVNHGDTLTATATAMARADQEGAQEIVCNVTLGGERREARENLTVFSFLGPILNLSEPSAPEGSTVTVSCMAGARVQVTLDGVPAAAPGQPAQLQLNATESDDGRSFFCSATLEVDGEFFHRNSSVQLRVLYGPKIDRATCPQHLKWKDKTRHVLQCQARGNPHPELRCLKEGSSREVPVGIPFFVNVTHNGTYQCQASSSRGRYTLVVVMDIEAGNSHFVLVFLAVLVTLGVVTVVVALMYVFREHKRSGRYHVRQESTSLPLTSMQPTEAMGEEPSTAE

SEQ ID NO: 81 - protein, 13 aa, Pongo pygmeaus
RSDPGSKLAGTWL

SEQ ID NO: 82 - DNA, 1554 bp, Macaca mulatta
caggagttcctgctgcgggtggagccccagaaccctgtgtttcctgctggagggtccctgttggtgaactgcagtactgattgccccagctctaagaaaatcatcttggagacgtccctatcaaaggagctggtggacaatggcacaggctgggcagccttccagctcagcaacgtgactggcaacagtcggatcctctgttcagggtactgcaatggctcccagataacaggcttctctgacatcaccgtgtacagcctcccggagcgcgtggagctggcacccctgcctccttggcagccggtgggccagaacttgatcctgcgctgccaagtggaaggtgggtcgccccgcaccagcctcacggtggtgctgctccgctgggagaaggagctgacccggcagccagcagtgggggagccagcagaggtcaataccactgtgctgaccagcagagaggaccacggagcccatttctcatgccgcacagaactggacatgaagccccaggggctggaactcttccggaacacctcagccccccgccaactccgaacctttgccctgccggtgacccccccgcgcctcgtggccccccggttcttggaggtggaaaagtcgtggccggtgaactgcactctagatgggctttttccagcctcagaggcccaggtctacctggcactgggggaccagatgctgaatgcgacagtcatgaaccacggggacatgctaacggccacagccacagccacagcgcgcgcagatcaggagggtgcgcgggaaatcgtctgcaacgtgatcctagggggcgagagactggagacccgggagaacttgacggtctttagcttcctaggacccattctgaacctgagcgagcccagcgcccccgaggggtccacagtgaccgtgagctgcatggctggggctcgagtccaggtaacgctggacggagttccagccgcggccccggggcagccagctcaacttcagttaaatgctaccgagagtgacgacggacgcaacttcttctgcagtgccactctcgaggtggacggcgagttcttgtgtaggaacagtagcgtccagctgcgtgtcctgtatggtcccaaaattgaccgagccacatgcccccagcacttgaagtggaaagacaaaacgagacacgtcctgcagtgccaagccaggggcaacccgtacccccagctgcggtgtttgaaggaaggctccaaccgggaggtgccggtggggatcccgttcttcgtcaatgtaacacataatggcacttatcaatgccaagcgtccagctcacgaggcaaatacaccctggtcgtggtgatggatattgaggctccgaagtcccactttgtccctgtcttcttggcggtgttagtgaccctgggcgtggtgactgtcgtagtggccttaatgtacgtcttcaaggagcataaacggagcggcaggtaccatgttaggcaggagagcacctctctgcccctcacgtctatgcagccgacagaggcaatgggggaagaaccgtccagagctgag

SEQ ID NO: 83 - DNA, 1554 bp, Macaca mulatta; identical to SEQ ID NO: 82
  CDS (1)..(1554)

SEQ ID NO: 84 - protein, 518 aa, Macaca mulatta
QEFLLRVEPQNPVFPAGGSLLVNCSTDCPSSKKIILETSLSKELVDNGTGWAAFQLSNVTGNSRILCSGYCNGSQITGFSDITVYSLPERVELAPLPPWQPVGQNLILRCQVEGGSPRTSLTVVLLRWEKELTRQPAVGEPAEVNTTVLTSREDHGAHFSCRTELDMKPQGLELFRNTSAPRQLRTFALPVTPPRLVAPRFLEVEKSWPVNCTLDGLFPASEAQVYLALGDQMLNATVMNHGDMLTATATATARADQEGAREIVCNVILGGERLETRENLTVFSFLGPILNLSEPSAPEGSTVTVSCMAGARVQVTLDGVPAAAPGQPAQLQLNATESDDGRNFFCSATLEVDGEFLCRNSSVQLRVLYGPKIDRATCPQHLKWKDKTRHVLQCQARGNPYPQLRCLKEGSNREVPVGIPFFVNVTHNGTYQCQASSSRGKYTLVVVMDIEAPKSHFVPVFLAVLVTLGVVTVVVALMYVFKEHKRSGRYHVRQESTSLPLTSMQPTEAMGEEPSRAE